(19) United States
(12) Patent Application Publication
(10) Pub. No.: US 2024/0027469 A1
(43) Pub. Date: Jan. 25, 2024

(54) BIOLOGICAL VESICLES DISPLAYING CELL SURFACE PROTEINS AND METHODS RELATED TO SAME

(71) Applicant: Genentech, Inc., South San Francisco, CA (US)

(72) Inventors: Nadia MARTINEZ-MARTIN, South San Francisco, CA (US); Sean M. PETERSON, South San Francisco, CA (US); Shengya CAO, South San Francisco, CA (US)

(21) Appl. No.: 18/326,476

(22) Filed: May 31, 2023

Related U.S. Application Data
(63) Continuation of application No. PCT/US2021/061120, filed on Nov. 30, 2021.
(60) Provisional application No. 63/227,039, filed on Jul. 29, 2021.
(60) Provisional application No. 63/212,021, filed on Jun. 17, 2021.
(60) Provisional application No. 63/120,167, filed on Dec. 1, 2020.

(51) Int. Cl. G01N 33/68

(52) U.S. Cl. CPC G01N 33/6845

(57) ABSTRACT

Provided herein are biological vesicles displaying cell surface proteins, as well as methods of using such vesicles to identify and characterize protein-protein interactions.

### CROSS-REFERENCE TO RELATED APPLICATIONS

This application is a continuation of International Patent Application No. PCT/US2021/061120, filed on Nov. 30, 2021, which claims benefit to U.S. Patent Application No. 63/120,167, filed on Dec. 1, 2020; U.S. Patent Application No. 63/212,021, filed on Jun. 17, 2021; and U.S. Patent Application No. 63/227,039, filed on Jul. 29, 2021, the entire contents of each of which are incorporated herein by reference in their entirety.

### SEQUENCE LISTING

The instant application contains a Sequence Listing which has been submitted electronically in XML format and is hereby incorporated by reference in its entirety. Said XML copy, created on May 12, 2023 is named 50474-231005_Sequence_Listing_5_10_23.xml and is 2,293 bytes in size.

### FIELD OF THE INVENTION

Provided herein are biological vesicles displaying cell surface proteins, as well as methods of using such vesicles to identify and characterize protein-protein interactions.

### BACKGROUND

Plasma membrane-expressed proteins and their interactors play a prominent role in the initiation of signal transduction to the cytosol of the cell, and thus are key regulators of most biological pathways. Increasing evidence demonstrates that receptors have a complex landscape of interacting partners in the extracellular milieu that directly influence their biological functions. As a result, dysregulation of receptor-ligand crosstalk often underlies pathology and disease progression. However, receptor interaction networks remain understudied, due to of the biochemical challenges associated with maintaining membrane proteins in their native conformation proteins and typically weak interactions among receptors.

Thus, there is an unmet need for methods and compositions for the identification of interactions between cell surface proteins, as well as novel modulators of such interactions and methods of using the same.

### SUMMARY OF THE INVENTION

In one aspect, the disclosure features a method for identifying a protein-protein interaction, the method comprising (a) providing a collection of target polypeptides that are immobilized on one or more solid surfaces; (b) contacting the collection of step (a) with a biological vesicle (BV) comprising a heterologous membrane-associated protein and a membrane-budding agent under conditions permitting the binding of the heterologous membrane-associated protein and at least one of the target polypeptides, wherein the heterologous membrane-associated protein is expressed at or above a threshold level on the surface of the BV; and (c) detecting an interaction between the heterologous membrane-associated protein and the at least one target polypeptide, thereby identifying a protein-protein interaction.

In some aspects, one or more of the target polypeptides is immobilized to a distinct location on the one or more solid surfaces.

In some aspects, detecting an interaction comprises detecting a signal at a location on the solid surface that is above a threshold level.

In some aspects, the membrane-budding agent is selected from the group consisting of a HIV gag protein, Acyl.Hrs, ARRDC1, and ARF6. In some aspects, the membrane-budding agent is a HIV gag protein.

In some aspects, the membrane-budding agent further comprises a detectable marker, and detecting an interaction comprises detecting a level of the detectable marker at a location on the solid surface that is above a threshold level. In some aspects, the detectable marker is an enzyme that produces a fluorescent signal in the presence of a substrate. In some aspects, the enzyme is Renilla luciferase (Rluc) and the substrate is Rluc substrate.

In some aspects, the BV comprises a membrane marker, and detecting an interaction comprises detecting a level of the membrane marker at a location on the solid surface that is above a threshold level.

In some aspects, the membrane marker is a cholesterol marker. In some aspects, the cholesterol marker is AMPLEX™ Red.

In some aspects, the interaction is a transient interaction.

In some aspects, the interaction is a low-affinity interaction.

In some aspects, the heterologous membrane-associated protein is a full-length protein.

In some aspects, the heterologous membrane-associated protein comprises a protein fragment, a tag, and an anchor.

In some aspects, the anchor tethers the protein fragment to the surface of a membrane of a BV.

In some aspects, the anchor is a glycosylphosphatidyl-inositol (GPI) polypeptide.

In some aspects, the tag can be directly or indirectly visualized. In some aspects, the tag comprises a moiety that can be detected using an antibody or an antibody fragment. In some aspects, the tag is a glycoprotein D (gD) polypeptide.

In some aspects, the expression level of the heterologous membrane-associated protein is determined using a biolayer interferometry (BLI) assay.

In some aspects, the tag is a gD polypeptide, expression of the heterologous membrane-associated protein is detected using an anti-gD antibody, and the threshold level is a shift of 1.5 nm, as measured using the BLI assay at 30° C.

In some aspects, the tag comprises a fluorescent protein.

In some aspects, the heterologous membrane-associated protein is a transmembrane receptor or a fragment thereof. In some aspects, the receptor is a single-pass transmembrane (STM) receptor.

In some aspects, the protein fragment is an extracellular domain.

In some aspects, each member of the collection of target polypeptides is an Fc-tagged extracellular domain, and wherein the solid surface is coated with protein A.

In some aspects, the collection of target polypeptides comprises the extracellular domains of at least 25% of the proteins of Table 4. In some aspects, the collection of target polypeptides comprises the extracellular domains of at least 50% of the proteins of Table 4. In some aspects, the collection of target polypeptides comprises the extracellular domains of at least 75% of the proteins of Table 4. In some aspects, the collection of target polypeptides comprises the extracellular domains of at least 90% of the proteins of Table 4. In some aspects, the collection of target polypeptides comprises the extracellular domains of all of the proteins of Table 4.

In another aspect, the disclosure features a BV comprising (a) a heterologous membrane-associated protein comprising a protein fragment, a tag, and an anchor, wherein the heterologous membrane-associated protein is present on the outer face of the BV and (b) a membrane-budding agent.

In another aspect, the disclosure features a BV comprising (a) a heterologous membrane-associated protein comprising a protein fragment, a tag, and an anchor, wherein the heterologous membrane-associated protein is present on the outer face of the BV and (b) a membrane-budding agent, the BV being produced by a process comprising (i) providing a parent cell that has been modified to express the heterologous membrane-associated protein and the membrane-budding agent and (ii) isolating the BV from the parent cell.

In some aspects, the membrane-budding agent is selected from the group consisting of a HIV gag protein, Acyl.Hrs, ARRDC1, and ARF6. In some aspects, the membrane-budding agent is a HIV gag protein.

In some aspects, the anchor tethers the protein fragment to the surface of a lipid membrane of a BV. In some aspects, the anchor is a GPI polypeptide.

In some aspects, the tag can be directly or indirectly visualized. In some aspects, the tag comprises a moiety that can be detected using an antibody or an antibody fragment. In some aspects, the tag is a gD polypeptide.

In some aspects, the tag comprises a fluorescent protein.

In some aspects, the protein fragment is an extracellular domain of a transmembrane receptor. In some aspects, the transmembrane receptor is a STM receptor.

In some aspects, the BV produces a shift that is at or above a threshold level when contacted with an antibody against the tag, as measured using a BLI assay.

In some aspects, the tag is a gD polypeptide, the antibody is an anti-gD antibody, and the threshold level is a shift of 1.5 nm, as measured using the BLI assay at 30° C.

In some aspects, the membrane-budding agent comprises a detectable marker. In some aspects, the detectable marker is an enzyme that produces a fluorescent signal in the presence of a substrate. In some aspects, the enzyme is Rluc and the substrate is Rluc substrate.

In some aspects, the BV comprises a membrane marker. In some aspects, the membrane marker is a cholesterol marker. In some aspects, the cholesterol marker is AMPLEX™ Red.

In some aspects, the BV is produced by a mammalian parent cell. In some aspects, the BV is an extracellular vesicle (EV). In some aspects, the BV is an exosome or a microvesicle. In some aspects, the BV is a virus-like particle (VLP).

In some aspects, the parent cell has been transfected with a plasmid encoding the heterologous membrane-associated protein and a plasmid encoding the membrane-budding agent.

In another aspect, the disclosure features a method of identifying a modulator of the interaction between a protein of Table 1 and a protein of Table 2, the method comprising (a) providing a candidate modulator; (b) contacting a protein of Table 1 with a protein of Table 2 in the presence or absence of the candidate modulator under conditions permitting the binding of the protein of Table 1 to the protein of Table 2, wherein the protein of Table 1 and the protein of Table 2 are reported to interact in Table 3; and (c) measuring the binding of the protein of Table 1 to the protein of Table 2, wherein an increase or decrease in binding in the presence of the candidate modulator relative to binding in the absence of the candidate modulator identifies the candidate modulator as a modulator of the interaction between the protein of Table 1 and the protein of Table 2.

In another aspect, the disclosure features a method of identifying a modulator of a downstream activity of a protein of Table 1, the method comprising (a) providing a candidate modulator; (b) contacting the protein of Table 1 with a protein of Table 2 in the presence or absence of the candidate modulator under conditions permitting the binding of the protein of Table 1 to the protein of Table 2, wherein the protein of Table 1 and the protein of Table 2 are reported to interact in Table 3; and (c) measuring a downstream activity of the protein of Table 1, wherein a change in the downstream activity in the presence of the candidate modulator relative to the downstream activity in the absence of the candidate modulator identifies the candidate modulator as a modulator of the downstream activity of the protein of Table 1.

In another aspect, the disclosure features a method of identifying a modulator of a downstream activity of a protein of Table 2, the method comprising (a) providing a candidate modulator; (b) contacting the protein of Table 2 with a protein of Table 1 in the presence or absence of the candidate modulator under conditions permitting the binding of the protein of Table 2 to the protein of Table 1, wherein the protein of Table 1 and the protein of Table 2 are reported to interact in Table 3; and (c) measuring a downstream activity of the protein of Table 2, wherein a change in the downstream activity in the presence of the candidate modulator relative to the downstream activity in the absence of the candidate modulator identifies the candidate modulator as a modulator of the downstream activity of the protein of Table 2.

In some aspects, the increase or decrease in binding is at least 70%, as measured by a surface plasmon resonance (SPR) assay, a BLI assay, or an enzyme-linked immunosorbent assay (ELISA).

In some aspects, the modulator is an inhibitor of the downstream activity of the protein of Table 1 or Table 2. In some aspects, the modulator is an activator of the downstream activity of the protein of Table 1 or Table 2.

In some aspects, the change in the downstream activity is a decrease in the amount, strength, or duration of the downstream activity. In some aspects, the change in the downstream activity is an increase in the amount, strength, or duration of the downstream activity.

In some aspects, the modulator is a small molecule, an antibody or antigen-binding fragment thereof, a peptide, a mimic, an antisense oligonucleotide, or a small interfering RNA (siRNA).

In some aspects, the antigen-binding fragment is a bis-Fab, an Fv, a Fab, a Fab′-SH, a F(ab′)₂, a diabody, a linear antibody, an scFv, an ScFab, a VH domain, or a VHH domain.

In some aspects, the antibody or antigen-binding fragment thereof binds the protein of Table 1. In some aspects, the antibody or antigen-binding fragment thereof binds the protein of Table 2.

In some aspects, the protein of Table 1 is LRRC15. In some aspects, the protein of Table 2 is TEM1. In some aspects, the downstream activity is tumor growth. In some aspects, tumor growth is decreased in the presence of the modulator. In some aspects, tumor growth is decreased by at least 20%, as measured in a tumor growth assay.

In some aspects, the modulator is an antibody or antigen-binding fragment thereof targeting LRRC15.

In some aspects, the modulator is an antibody or antigen-binding fragment thereof targeting TEM1.

In another aspect, the disclosure features a method of identifying a modulator of the interaction between LRRC15 and TEM1, the method comprising (a) providing a candidate modulator; (b) contacting LRRC15 with TEM1 in the presence or absence of the candidate modulator under conditions permitting the binding of LRRC15 to TEM1; and (c) measuring the binding of LRRC15 to TEM1, wherein an increase or decrease in binding in the presence of the candidate modulator relative to binding in the absence of the candidate modulator identifies the candidate modulator as a modulator of the interaction between LRRC15 and TEM1.

In another aspect, the disclosure features a method of identifying a modulator of a downstream activity of LRRC15, the method comprising (a) providing a candidate modulator; (b) contacting LRRC15 with TEM1 in the presence or absence of the candidate modulator under conditions permitting the binding of LRRC15 to TEM1; and (c) measuring a downstream activity of LRRC15, wherein a change in the downstream activity in the presence of the candidate modulator relative to the downstream activity in the absence of the candidate modulator identifies the candidate modulator as a modulator of the downstream activity of LRRC15.

In another aspect, the disclosure features a method of identifying a modulator of a downstream activity of TEM1, the method comprising (a) providing a candidate modulator; (b) contacting TEM1 with LRRC15 in the presence or absence of the candidate modulator under conditions permitting the binding of TEM1 to LRRC15; and (c) measuring a downstream activity of TEM1, wherein a change in the downstream activity in the presence of the candidate modulator relative to the downstream activity in the absence of the candidate modulator identifies the candidate modulator as a modulator of the downstream activity of TEM1.

In some aspects, the increase or decrease in binding is at least 70%, as measured by an SPR assay, a BLI assay, or ELISA.

In some aspects, the downstream activity is tumor growth.

In some aspects, tumor growth is decreased in the presence of the modulator. In some aspects, tumor growth is decreased by at least 20%, as measured in a tumor growth assay.

In another aspect, the disclosure features a method for identifying a biological vesicle (BV) having an altered binding profile, the method comprising (a) providing a collection of target polypeptides that are immobilized on one or more solid surfaces; (b) contacting the collection of step (a) with a BV of interest; (c) detecting an interaction between the BV of interest and the at least one target polypeptide, thereby identifying an interaction profile; and (d) comparing the interaction profile of the BV of interest to the interaction profile of a control BV, wherein a difference between the interaction profile of the BV of interest and the interaction profile of the control BV identifies the BV of interest as one having an altered binding profile.

In some aspects, the collection of target polypeptides comprises the extracellular domains of at least 25% of the proteins of Table 4. In some aspects, the collection of target polypeptides comprises the extracellular domains of at least 50% of the proteins of Table 4. In some aspects, the collection of target polypeptides comprises the extracellular domains of at least 75% of the proteins of Table 4. In some aspects, the collection of target polypeptides comprises the extracellular domains of at least 90% of the proteins of Table 4. In some aspects, the collection of target polypeptides comprises the extracellular domains of all of the proteins of Table 4.

In some aspects, the BV of interest is an engineered BV.

In some aspects, the BV of interest is derived from a sample from a subject. In some aspects, the BV of interest and the control BV are derived from different tissues or different cell types. In some aspects, the BV of interest is derived from a diseased tissue and the control BV is derived from healthy tissue.

In another aspect, the disclosure features a protein complex comprising (a) a BV comprising a heterologous membrane-associated protein and a membrane-budding agent and (b) a target polypeptide, wherein the heterologous membrane-associated protein and the target polypeptide are bound to one another.

In another aspect, the disclosure features a method of identifying a modulator of the interaction between a protein of Table 5 and a protein of Table 6, the method comprising (a) providing a candidate modulator; (b) contacting a protein of Table 5 with a protein of Table 6 in the presence or absence of the candidate modulator under conditions permitting the binding of the protein of Table 5 to the protein of Table 6, wherein the protein of Table 5 and the protein of Table 6 are reported to interact in Table 7; and (c) measuring the binding of the protein of Table 5 to the protein of Table 6, wherein an increase or decrease in binding in the presence of the candidate modulator relative to binding in the absence of the candidate modulator identifies the candidate modulator as a modulator of the interaction between the protein of Table 5 and the protein of Table 6.

In another aspect, the disclosure features a method of identifying a modulator of a downstream activity of a protein of Table 5, the method comprising (a) providing a candidate modulator; (b) contacting the protein of Table 5 with a protein of Table 6 in the presence or absence of the candidate modulator under conditions permitting the binding of the protein of Table 5 to the protein of Table 6, wherein the protein of Table 5 and the protein of Table 6 are reported to interact in Table 7; and (c) measuring a downstream activity of the protein of Table 5, wherein a change in the downstream activity in the presence of the candidate modulator relative to the downstream activity in the absence of the candidate modulator identifies the candidate modulator as a modulator of the downstream activity of the protein of Table 5.

In another aspect, the disclosure features a method of identifying a modulator of a downstream activity of a protein of Table 6, the method comprising (a) providing a candidate modulator; (b) contacting the protein of Table 6 with a protein of Table 5 in the presence or absence of the candidate modulator under conditions permitting the binding of the protein of Table 6 to the protein of Table 5, wherein the protein of Table 5 and the protein of Table 6 are reported to interact in Table 7; and (c) measuring a downstream activity of the protein of Table 6, wherein a change in the downstream activity in the presence of the candidate modulator relative to the downstream activity in the absence of the candidate modulator identifies the candidate modulator as a modulator of the downstream activity of the protein of Table 6.

In some aspects, the increase or decrease in binding is at least 70%, as measured by a surface plasmon resonance (SPR) assay, a BLI assay, or an enzyme-linked immunosorbent assay (ELISA).

In some aspects, the modulator is an inhibitor of the downstream activity of the protein of Table 5 or Table 6.

In some aspects, the modulator is an activator of the downstream activity of the protein of Table 5 or Table 6.

In some aspects, the change in the downstream activity is a decrease in the amount, strength, or duration of the downstream activity. In some aspects, the change in the downstream activity is an increase in the amount, strength, or duration of the downstream activity.

In some aspects, the modulator is a small molecule, an antibody or antigen-binding fragment thereof, a peptide, a mimic, an antisense oligonucleotide, or a small interfering RNA (siRNA).

In some aspects, the antigen-binding fragment is a bis-Fab, an Fv, a Fab, a Fab′-SH, a F(ab′)₂, a diabody, a linear antibody, an scFv, an ScFab, a VH domain, or a VHH domain.

In some aspects, the antibody or antigen-binding fragment thereof binds the protein of Table 5.

In some aspects, the antibody or antigen-binding fragment thereof binds the protein of Table 6.

In some aspects, the protein of Table 5 is ADGRB1.

In some aspects, the protein of Table 6 is PD-L1.

In some aspects, the downstream activity is tumor growth. In some aspects, tumor growth is decreased in the presence of the modulator. In some aspects, tumor growth is decreased by at least 20%, as measured in a tumor growth assay.

In some aspects, the modulator is an antibody or antigen-binding fragment thereof targeting PD-L1.

In some aspects, the protein of Table 6 is ICOSLG.

In some aspects, the downstream activity is T cell activation. In some aspects, T cell activation is increased in the presence of the modulator. In some aspects, T cell activation is increased by at least 20%.

In some aspects, the modulator is an antibody or antigen-binding fragment thereof targeting ICOSLG.

In some aspects, the modulator is an antibody or antigen-binding fragment thereof targeting ADGRB1.

In another aspect, the disclosure features a method of identifying a modulator of the interaction between PD-L1 and ADGRB1, the method comprising (a) providing a candidate modulator; (b) contacting PD-L1 with ADGRB1 in the presence or absence of the candidate modulator under conditions permitting the binding of PD-L1 to ADGRB1; and (c) measuring the binding of PD-L1 to ADGRB1, wherein an increase or decrease in binding in the presence of the candidate modulator relative to binding in the absence of the candidate modulator identifies the candidate modulator as a modulator of the interaction between PD-L1 and ADGRB1.

In another aspect, the disclosure features a method of identifying a modulator of a downstream activity of PD-1, the method comprising (a) providing a candidate modulator; (b) contacting PD-L1 with ADGRB1 in the presence or absence of the candidate modulator under conditions permitting the binding of PD-L1 to ADGRB1; and (c) measuring a downstream activity of PD-L1, wherein a change in the downstream activity in the presence of the candidate modulator relative to the downstream activity in the absence of the candidate modulator identifies the candidate modulator as a modulator of the downstream activity of PD-1.

In another aspect, the disclosure features a method of identifying a modulator of a downstream activity of ADGRB1, the method comprising (a) providing a candidate modulator; (b) contacting ADGRB1 with PD-L1 in the presence or absence of the candidate modulator under conditions permitting the binding of ADGRB1 to PD-L1; and (c) measuring a downstream activity of ADGRB1, wherein a change in the downstream activity in the presence of the candidate modulator relative to the downstream activity in the absence of the candidate modulator identifies the candidate modulator as a modulator of the downstream activity of ADGRB1.

In some aspects, the increase or decrease in binding is at least 70%, as measured by an SPR assay, a BLI assay, or ELISA.

In some aspects, the downstream activity is tumor growth. In some aspects, tumor growth is decreased in the presence of the modulator. In some aspects, tumor growth is decreased by at least 20%, as measured in a tumor growth assay.

In another aspect, the disclosure features a method of identifying a modulator of the interaction between ICOSLG and ADGRB1, the method comprising (a) providing a candidate modulator; (b) contacting ICOSLG with ADGRB1 in the presence or absence of the candidate modulator under conditions permitting the binding of ICOSLG to ADGRB1; and (c) measuring the binding of ICOSLG to ADGRB1, wherein an increase or decrease in binding in the presence of the candidate modulator relative to binding in the absence of the candidate modulator identifies the candidate modulator as a modulator of the interaction between ICOSLG and ADGRB1.

In another aspect, the disclosure features a method of identifying a modulator of a downstream activity of ICOSLG, the method comprising (a) providing a candidate modulator; (b) contacting ICOSLG with ADGRB1 in the presence or absence of the candidate modulator under conditions permitting the binding of ICOSLG to ADGRB1; and (c) measuring a downstream activity of ICOSLG, wherein a change in the downstream activity in the presence of the candidate modulator relative to the downstream activity in the absence of the candidate modulator identifies the candidate modulator as a modulator of the downstream activity of ICOSLG.

In another aspect, the disclosure features a method of identifying a modulator of a downstream activity of ADGRB1, the method comprising (a) providing a candidate modulator; (b) contacting ADGRB1 with ICOSLG in the presence or absence of the candidate modulator under conditions permitting the binding of ADGRB1 to ICOSLG; and (c) measuring a downstream activity of ADGRB1, wherein a change in the downstream activity in the presence of the candidate modulator relative to the downstream activity in the absence of the candidate modulator identifies the candidate modulator as a modulator of the downstream activity of ADGRB1.

In some aspects, the increase or decrease in binding is at least 70%, as measured by an SPR assay, a BLI assay, or ELISA.

In some aspects, the downstream activity is T cell activation. In some aspects, T cell activation is decreased in the presence of the modulator. In some aspects, T cell activation is increased by at least 20%.

In another aspect, the disclosure features a method for characterizing an interaction profile of a cell line, the method comprising (a) modifying the cell line to comprise a membrane-budding agent; and (b) characterizing an interaction profile of a biological vesicle (BV) produced by the cell line.

In another aspect, the disclosure features a method for characterizing an interaction profile of a cell line that has been modified to comprise a membrane-budding agent, the method comprising characterizing an interaction profile of a BV produced by the cell line.

In another aspect, the disclosure features a method for identifying a change in the interaction profile of a cell line, the method comprising (a) modifying the cell line to comprise a membrane-budding agent; (b) characterizing an interaction profile of a BV produced by the cell line at a first time point; (c) characterizing an interaction profile of a BV produced by the cell line at a second time point; and (d) comparing the interaction profile of the BV produced at the first time point to that of the BV produced at the second time point, wherein a difference between the interaction profile of the BV produced at the first time point and that of the BV produced at the second time point identifies a change in the interaction profile of the cell line.

In another aspect, the disclosure features a method for identifying a change in the interaction profile of a cell line that has been modified to comprise a membrane-budding agent, the method comprising (a) characterizing an interaction profile of a BV produced by the cell line at a first time point; (b) characterizing an interaction profile of a BV produced by the cell line at a second time point; and (c) comparing the interaction profile of the BV produced at the first time point to that of the BV produced at the second time point, wherein a difference between the interaction profile of the BV produced at the first time point and that of the BV produced at the second time point identifies a change in the interaction profile of the cell line.

In some aspects, the cell line is a mammalian cell line. In some aspects, the mammalian cell line is an immune cell line, a neuronal cell line, or a fibroblast cell line. In some aspects, the immune cell line comprises one or more of T-cells, B-cells, or monocytes.

In some aspects, the method comprises exposing the cell line to a stimulus following the first time point and before the second time point.

In some aspects, the stimulus is a condition or agent that induces signaling. In some aspects, the stimulus is a condition or agent that induces a disease-related state. In some aspects, the cell line is an immune cell line and the disease-related state is immune exhaustion.

In some aspects, the stimulus is a condition or agent that induces differentiation.

In some aspects, the method further comprises characterizing an interaction profile of a BV produced by the cell line at one or more additional time points.

In another aspect, the disclosure features a method for identifying a difference in the interaction profiles of two cell lines, the method comprising (a) modifying each of the cell lines to comprise a membrane-budding agent; (b) characterizing an interaction profile of a BV produced by the first cell line; (c) characterizing an interaction profile of a BV produced by the second cell line; and (d) comparing the interaction profile of the BV produced at the first cell line to that of the BV produced by the second cell line, wherein a difference between the interaction profile of the BV produced by the first cell line and that of the BV produced by the second cell line identifies a difference in the surface protein profiles of two cell lines.

In another aspect, the disclosure features a method for identifying a difference in the interaction profiles of two cell lines that have been modified to comprise a membrane-budding agent, the method comprising (a) characterizing an interaction profile of a BV produced by the first cell line; (b) characterizing an interaction profile of a BV produced by the second cell line; and (c) comparing the interaction profile of the BV produced at the first cell line to that of the BV produced by the second cell line, wherein a difference between the interaction profile of the BV produced by the first cell line and that of the BV produced by the second cell line identifies a difference in the surface protein profiles of two cell lines.

In some aspects, expression of the membrane-budding agent is inducible.

In some aspects, characterizing the interaction profile of the BV comprises determining a level of one or more membrane-associated proteins of interest on the BV.

In some aspects, characterizing the interaction profile of the BV comprises determining a level of one or more receptors of interest on the BV.

In some aspects, characterizing the interaction profile of the BV is performed using a method comprising (a) providing a collection of target polypeptides that are immobilized on one or more solid surfaces; (b) contacting the collection of target polypeptides in step (a) with the BV; and (c) detecting an interaction between the BV and the at least one target polypeptide of the collection of target polypeptides, thereby identifying an interaction profile.

In some aspects, the collection of target polypeptides comprises the extracellular domains of at least 25% of the proteins of Table 4. In some aspects, the collection of target polypeptides comprises the extracellular domains of at least 50% of the proteins of Table 4. In some aspects, the collection of target polypeptides comprises the extracellular domains of at least 75% of the proteins of Table 4. In some aspects, the collection of target polypeptides comprises the extracellular domains of at least 90% of the proteins of Table 4. In some aspects, the collection of target polypeptides comprises the extracellular domains of all of the proteins of Table 4.

In some aspects, the method further comprises characterizing a cytoplasmic protein profile of the BV.

In some aspects, the membrane-budding agent is selected from the group consisting of a HIV gag protein, Acyl.Hrs, ARRDC1, and ARF6.

In another aspect, the disclosure features a BV comprising a heterologous membrane-budding agent, wherein the BV is produced by a process comprising (i) providing a parent cell line that has been modified to express the membrane-budding agent under inducible control; (ii) inducing expression of the membrane-budding agent, and (iii) isolating the BV from the parent cell line.

In some aspects, the membrane-budding agent is selected from the group consisting of a HIV gag protein, Acyl.Hrs, ARRDC1, and ARF6.

In some aspects, the parent cell line is a mammalian cell line.

In some aspects, the BV is an extracellular vesicle (EV).

In another aspect, the disclosure features a method for assessing an enzymatic activity of a membrane-associated protein, the method comprising conducting an assay for enzymatic activity on a BV comprising the protein.

In some aspects, the membrane-associated protein is a peptidase and the assay for enzymatic activity is an assay for peptidase activity.

In some aspects, the membrane-associated protein is a protease and the assay for enzymatic activity is an assay for protease activity.

In some aspects, the membrane-associated protein is a kinase and the assay for enzymatic activity is an assay for kinase activity.

In some aspects, the membrane-associated protein is a phosphatase and the assay for enzymatic activity is an assay for phosphatase activity.

In some aspects, the membrane-associated protein is endogenous to a parent cell from which the BV is derived.

In some aspects, the membrane-associated protein is heterologous to a parent cell from which the BV is derived. In some aspects, the heterologous membrane-associated protein is a full-length protein. In some aspects, the heterologous membrane-associated protein comprises a protein fragment, a tag, and an anchor. In some aspects, the anchor tethers the protein fragment to the surface of a membrane of the BV. In some aspects, the anchor is a glycosylphosphatidyl-inositol (GPI) polypeptide.

In another aspect, the disclosure features a method of purifying a BV from a culture medium or a sample from a subject, the method comprising contacting a BV with a solid surface comprising one or more of the proteins of Table 8 or Table 9, wherein the one or more proteins of Table 8 or Table 9 have been modified to comprise an Fc region.

In some aspects, the sample from the subject is a urine sample, a blood sample, or a digested tissue sample

In some aspects, the solid surface is a column comprising Protein A-functionalized beads and the method comprises flowing the conditioned media comprising the one or more of the proteins of Table 8 or Table 9 over the column.

In some aspects, the method further comprises flowing the culture medium comprising the BV over the column.

In some aspects, the method further comprises eluting the BV.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

FIG. 1A is a schematic diagram showing the isolation of receptor-expressing extracellular vesicles (EVs) from cell culture. EXPI293F™ cells were transiently transfected with a plasmid encoding a receptor of interest and a plasmid encoding an HIV gag protein fused to Renilla luciferase (Rluc). Cells and debris were separated from the EV-containing supernatant by centrifugation and filtration. A 50% sucrose cushion was used to remove small protein aggregates, and small vesicles were isolated.

FIG. 1B is a set of negative stain electron micrographs showing EVs prepared with (right panel) and without (left panel) the sucrose cushion step. EV preparations were diluted to the same protein concentration prior to electron microscopy. Arrows point to representative EVs in the sample.

FIG. 1C is a pair of graphs showing the size distribution (in nm) of EVs carrying a full-length (FL) PVR protein (left panel) or a protein comprising the PVR ectodomain, a glycoprotein D (gD) tag, and a glycosylphosphatidylinositol (GPI) linker (gD-GPI) (right panel), as measured using nanoparticle tracking analysis. Five replicates are shown in each graph. Black line represents the mean; gray line represents the standard error of the mean. EVs are consistently about 120 nm in size.

FIG. 2A is a diagram showing transmembrane proteins embedded in the plasma membrane of the cell and in the EV membrane.

FIG. 2B is a diagram showing two experimental setups for EV-expressed receptors. Left: HIV gag proteins and full-length transmembrane receptors embedded in a membrane of the cell and in EV membranes. Right: HIV gag proteins and lipid-anchored ectodomains comprising gD-GPI tags embedded in a membrane of the cell and in EV membranes.

FIG. 2C is a graph showing the particle count of EVs in the 20-500 nm size range produced by parent cells that have been transformed with an HIV gag protein (With Gag) and control untransformed cells (No Gag), as measured using nanoparticle tracking analysis.

FIG. 2D is a graph showing the luminescence signal of Rluc in a 3-fold dilution series of an EV preparation produced from parent cells that were transformed with a plasmid encoding an HIV gag protein fused to Rluc.

FIG. 2E is a schematic diagram showing EVs expressing full-length PVR bound to the surface of a mammalian cell expressing a PVR ligand and a set of micrographs showing EVs bound to the surface of cells expressing the indicated full-length PVR ligands. EVs comprised gag-NeonGreen, and green represents direct fluorescence from the EVs. DNA of the mammalian cells is shown in blue.

FIG. 2F is a schematic diagram showing EVs expressing the PVR ectodomain with a gD-GPI tag bound to the surface of a mammalian cell expressing a PVR ligand and a set of micrographs showing EVs bound to the surface of cells expressing the indicated full-length PVR ligands. EVs comprised gag-NeonGreen, and green represents direct fluorescence from the EVs. DNA of the mammalian cells is shown in blue. Scale bar is 20 μm.

FIG. 2G is a schematic diagram and a graph showing the design and results of a biolayer interferometry (BLI) experiment. CD226-Fc or a control human IgG were attached to a sensor. The sensor was dipped into a solution comprising EVs expressing full-length (FL) PVR or gD-GPI PVR ectodomains or monomeric PVR protein (PVR monomer), and the BLI signal (in nm) was measured. Right panel is a zoom of the signal above 0 nm.

FIG. 3A is a schematic diagram showing the workflow of the RDIMIS (Receptor-Display In Membranes Interaction Screen) protocol. EVs are isolated from the conditioned media of cells expressing the receptor of interest alongside gag-luc. A library of single-pass transmembrane (STM) proteins, expressed as Fc-tagged ectodomains (ECD-Fc), are immobilized on plates. Receptor-EVs are screened against the collection of plate-bound STM proteins using a semi-automated workflow. EV binding to interacting ectodomains in the library is detected using luminescence.

FIG. 3B is a scatter plot showing the results of two independent RDIMIS screens (Repeat 1 and Repeat 2) testing for interaction between PVR gD-GPI EVs and the STM protein library.

FIG. 3C is a scatter plot showing the results of an RDIMIS screen testing for interaction between PVR gD-GPI EVs and the STM protein library (Repeat 2 of FIG. 3B) and an RDIMIS screen testing for interaction between full-length (FL) PVR EVs and the STM protein library.

FIG. 4A is a set of photomicrographs showing Western blots of whole cell lysates (Cells) or EVs expressing the full-length untagged receptors PD1, PD-L1, EPHA3, CD248, LRRC15, PVR, or PVRL1 and stained with an antibody specific for the receptor. Anti-tubulin (α-Tub) and anti-actin (α-Actin) staining are provided as controls.

FIG. 4B is a set of photomicrographs showing Western blots of whole cell lysates or EVs expressing the indicated gD-GPI tagged receptor ectodomains and stained with an antibody specific for the gD tag (α-gD). Anti-tubulin (α-Tub) and anti-actin (α-Actin) staining are provided as controls.

FIG. 4C is a set of negative stain electromicrographs showing selective anti-gD immunogold labeling of gD-GPI expressing vesicles.

FIG. 4D is a graph showing the design and results of a biolayer interferometry experiment. An anti-gD antibody was attached to a sensor. The sensor was incubated with EVs expressing the indicated gD-GPI ectodomains, and the BLI signal (in nm) was measured.

FIG. 5A is a scatter plot showing the results of an RDIMIS screen testing for interaction between PVR gD-GPI EVs and the STM protein library and an RDIMIS screen testing for interaction between PD-L1 gD-GPI EVs and the STM protein library. Screens are plotted against one another to differentiate receptor-specific hits (near either axis) from the generic vesicle binders common between the screens.

Hits whose signal is above the 98% quantile for each individual screen and for which there is at least a 4× enrichment for a specific screen are labeled. Other hits are identified as generic vesicle binders common between the screens.

FIG. 5B is a scatter plot showing the results of an RDIMIS screen testing for interaction between CD80 gD-GPI EVs and the STM protein library and an RDIMIS screen testing for interaction between CD276 gD-GPI EVs and the STM protein library. Receptor-specific hits are located near the axes. Hits whose signal is above the 98% quantile for each individual screen and for which there is at least a 4× enrichment for a specific screen are labeled. Other hits are identified as generic vesicle binders common between the screens.

FIG. 5C is a set of diagrams showing the overlap between the binding partners identified for PVR, PD-L1, CD80, and CD276 in the present study and interactions listed in the STRING, Bioplex and Biogrid databases. For PD-L1/CD274, no interactions with members of the STM library were present in the Bioplex database. No experimentally verified interactions were listed in STRING for CD276/B7-H3.

FIG. 6A is a scatter plot showing the results of an AVEXIS screen testing for interaction between LRRC15 ectodomain pentamers and the STM protein library. LRRC15 pentamer binding was not observed above background in the well containing the CD248 ectodomain (highlighted). Gray dots indicate positive control wells on each plate, in which stock pentamer was added but not washed away.

FIG. 6B is a set of diagrams showing a comparison between the hits identified herein using LRRC15 full-length (FL) or gD-GPI ectodomain in EVs and those represented in the Bioplex and Biogrid databases. No interaction with experimental evidence between LRRC15 and a STM protein in the library was represented in the STRING database.

FIG. 7A is a scatter plot showing the results of an RDIMIS screen testing for interaction between LRRC15 gD-GPI EVs and the STM protein library. Results are compared with PVR screen results shown in FIG. 3C. Hits whose signal is above the 98% quantile for each individual screen and for which there is at least a 4× enrichment for a specific screen are labeled. Other hits are identified as generic vesicle binders common between the screens.

FIG. 7B is a scatter plot showing the results of an RDIMIS screen testing for interaction between LRRC15 full-length EVs and the STM protein library. Results are compared with PVR screen results shown in FIG. 3C. Hits whose signal is above the 98% quantile for each individual screen and for which there is at least a 4× enrichment for a specific screen are labeled. Other hits are identified as generic vesicle binders common between the screens.

FIG. 8A is a scatter plot showing bulk RNA-seq expression levels (transcripts per million (TPM)) of LRRC15 (x-axis) and CD248 (y-axis) for head and neck squamous cell carcinoma. Each point represents a single patient sample. Spearman's rank correlation coefficient and significance values are given on the top right.

FIG. 8B is a scatter plot showing bulk RNA-seq expression levels (transcripts per million (TPM)) of LRRC15 (x-axis) and CD248 (y-axis) for breast invasive carcinoma. Each point represents a single patient sample. Spearman's rank correlation coefficient and significance values are given on the top right.

FIG. 8C is a pair of Uniform Manifold Approximation and Projection (UMAP) dimensionality reduction plots showing non-tumor cells from single-cell RNA-seq data of head and neck cancer patients. Cells are shaded by expression level of the indicated marker genes (left; LRRC15; right: CD248).

FIG. 9A is a scatter plot showing bulk RNA-seq expression levels (transcripts per million (TPM)) of LRRC15 (x-axis) and CD248 (y-axis) for pancreatic ductal adenocarcinoma (The Cancer Genome Atlas (TCGA) data).

FIG. 9B is a scatter plot showing bulk RNA-seq expression levels (transcripts per million (TPM)) of LRRC15 (x-axis) and CD248 (y-axis) for urothelial bladder carcinoma (The Cancer Genome Atlas (TCGA) data).

FIG. 9C is a pair of UMAP dimensionality reduction plots showing non-tumor cells from single-cell RNA-seq data of head and neck cancer patients. Cells are shaded by expression level of the indicated marker genes (left; DCN cancer-associated fibroblasts; right: RGS5 cancer-associated pericyte markers).

FIG. 10A is a scatter plot showing the results of an AVEXIS screen testing for interaction between CD248 ectodomain pentamers and the STM protein library. CD248 pentamer binding was not observed above background in the well containing the LRRC15 ectodomain (highlighted). Gray dots indicate positive control wells on each plate, in which stock pentamer was added but not washed away.

FIG. 10B is a graph showing the results of a surface plasmon resonance (SPR) assay in which LRRC15-Fc was captured by Protein A on the SPR chip and the indicated analytes were added. LRRC15 was loaded at either 5 μg/mL (red and green lines) or 50 μg/mL concentration. Analytes were loaded at 400 nM concentration.

FIG. 11A is a schematic diagram and a graph showing the design and results of a BLI experiment. CD248 was expressed as a recombinant protein and immobilized on a sensor. The sensor was contacted with a solution comprising LRRC15-Fc (500 nM) or EVs comprising LRRC15 full-length or gD-GPI LRRC15 (0.25 mg/ml), and the BLI signal (in nm) was measured.

FIG. 11B is a schematic diagram and a set of micrographs showing binding of EVs comprising gag-NeonGreen and full-length (FL) LRRC15, gD-GPI LRRC15 (LRRC15-gD), or an empty-vector control to cells transiently expressing full-length CD248 or gD-GPI CD248.

FIG. 11C is a schematic diagram and a set of micrographs showing binding of CD248 (as a tetramerized recombinant ectodomain) or an empty-vector control to cells transiently expressing full-length LRRC15 or gD-GPI LRRC15. Scale bar represents 20 μm.

FIG. 11D is a bar graph showing a quantification of EV binding in FIG. 11B based on NeonGreen signal levels (mean±standard error for three independent replicates).

FIG. 12A is a scatter plot showing a comparison of Renilla luciferase fluorescence and fluorescence of cholesterol detected using the AMPLEX™ Red Cholesterol Assay Kit (Thermo Fisher) (Cholesterol) as a readout for a dilution series of PD-L1 and PVR gD-GPI EVs harvested from EXPI293F™ cells transfected with gag-Rluc or untransfected cells.

FIG. 12B is a bar graph showing the relative signal levels from Renilla luciferase and the AMPLEX™ Red Cholesterol Assay Kit (Thermo Fisher) observed in a small-scale RDIMIS screen with the listed genes immobilized in wells and probed using PD-L1 gD-GPI EVs. Results for both readouts are normalized to the respective PDCD1 signals.

FIG. 12C is a scatter plot showing the results of an RDIMIS screen performed using cholesterol as a readout on EVs expressing gag-Rluc (x-axis) or vesicles harvested from untransfected cells (y-axis).

FIG. 13A is a set of photomicrographs showing Western blots of EVs expressing full-length (FL) or gD-GPI tagged ectodomains of the receptors PVR, PD-L1, CD276, CD80, and LRRC15 stained with an antibody specific for the gD tag (α-gD). α-Actin, α-PVR, and α-LRRC15 staining are also shown.

FIG. 13B is a schematic diagram and a graph showing the design and results of a BLI experiment. Biotinylated PD-L1 was incubated with a streptavidin BLI biosensor. This was then incubated with EVs expressing full-length or gD-GPI LRTM1 or a vector control, and the BLI signal (in nm) was measured.

FIG. 13C is a set of micrographs showing EVs bound to the surface of cells transiently expressing full-length or gD-GPI tagged PD-L1. EVs contained full-length or gD-GPI LRTM1 or a vector control. EVs comprised gag-NeonGreen, and green represents direct fluorescence from the EVs. DNA of the mammalian cells is shown in blue.

FIG. 13D is a schematic diagram and a graph showing the design and results of a BLI experiment. Biotinylated PD-L1 was incubated with a streptavidin BLI biosensor. This was then incubated with EVs expressing full-length or gD-GPI LRTM1 or a vector control in the presence or absence of different concentrations of Fc-tagged PD1 ectodomain or a human IgG control, and the BLI signal (in nm) was measured.

FIG. 14A is a scatter plot showing the results of an RDIMIS screen performed using EVs comprising PVR gD-GPI (Y axis) compared to “empty” EVs derived from cells transfected with a vector control (X axis).

FIG. 14B is a scatter plot showing the results of an RDIMIS screen performed using EVs comprising PD-L1 gD-GPI (Y axis) compared to “empty” EVs derived from cells transfected with a vector control (X axis).

FIG. 14C is a scatter plot showing the results of an RDIMIS screen performed using EVs comprising CD80 gD-GPI (Y axis) compared to “empty” EVs derived from cells transfected with a vector control (X axis).

FIG. 14D is a scatter plot showing the results of an RDIMIS screen performed using EVs comprising CD276 gD-GPI (Y axis) compared to “empty” EVs derived from cells transfected with a vector control (X axis).

FIG. 14E is a scatter plot showing the results of an RDIMIS screen performed using EVs comprising LRRC15 gD-GPI (Y axis) compared to “empty” EVs derived from cells transfected with a vector control (X axis).

FIG. 14F is a scatter plot showing the results of an RDIMIS screen performed using EVs comprising full-length LRRC15 (Y axis) compared to “empty” EVs derived from cells transfected with a vector control (X axis).

FIG. 15 is a network diagram showing the generic vesicle binders identified herein (green boxes) integrated with the IgSF Interactome's list of high confidence interactions (1) (blue edges) and the experimental and database list of interactions from STRING (2) (red edges) to identify potential interaction partners (blue boxes). The height of the boxes represent normalized expression values in HEK293 cells from The Cell Atlas (3) to estimate the expression of the potential binding partners in the EV parent cells and, therefore, the EVs themselves.

FIG. 16A is a set of scatter plots showing correlations and correlation coefficients for each of the RDIMIS screens performed. Screens were done in several batches: 1) PVR gD-GPI repeat 1, 2) PVR gD-GPI repeat 2 and PD-L1 gD-GPI, 3) CD80 gD-GPI and CD26 gD-GPI, 4) LRRC15 gD-GPI, LRRC15 FL and PVR FL, 5) Vesicle control which are EVs with no overexpressed receptor-of-interest.

FIG. 16B is a set of scatter plots showing the correlation between CD80 gD-GPI and PVR-FL screens. Two populations of generic vesicle binders are shown. Lower panel: zoomed-in view of x axis.

Right panel: correlation with generic vesicle binders removed.

FIG. 17A is a graph showing the results of a BLI experiment in which membranes were disrupted with the cholesterol binder Filipin III. CD248 monomer were loaded onto a NiNTA biosensor and incubated with LRRC15 gD-GPI EVs that had been pre-treated for 30 minutes at room temperature with filipin III. Empty vesicles or filipin III are shown as negative controls.

FIG. 17B is a graph showing the results of a BLI experiment in which membranes were disrupted with the cholesterol binder Filipin III. CD248 monomer were loaded onto a NiNTA biosensor and incubated with full-length LRRC15 EVs that had been pre-treated for 30 minutes at room temperature with filipin III. Empty vesicles or filipin III are shown as negative controls.

FIG. 17C is a graph showing binding of the EVs of FIG. 17A to an anti-gD antibody.

FIG. 17D is a graph showing the results of a BLI experiment in which membranes were disrupted with Methyl-beta cyclodextrin (MpβCD). CD248 monomer were loaded onto a NiNTA biosensor and incubated with LRRC15 gD-GPI EVs that had been pre-treated for 30 minutes at room temperature with filipin III. Empty vesicles or filipin III are shown as negative controls.

FIG. 17E is a graph showing the results of a BLI experiment in which membranes were disrupted with MpβCD. CD248 monomer were loaded onto a NiNTA biosensor and incubated with full-length LRRC15 EVs that had been pre-treated for 30 minutes at room temperature with filipin III. Empty vesicles or filipin III are shown as negative controls.

FIG. 17F is a graph showing binding of the EVs of FIG. 17D to an anti-gD antibody.

FIG. 18A is a scatter plot showing level of antibody surface staining (a.u.) for >500 multi-transmembrane receptors expressed on cells and a pair of photomicrographs showing representative cell surface staining for a low-expressing receptor (DRD2) and a high-expressing receptor (S1 PR1). Background staining is denoted by the line.

FIG. 18B is a scatter plot showing level of surface staining (a.u.) using an anti-FLAG antibody and fluorescence of a Venus tag (X-axis; total receptor (a.u.)) for >400 G protein-coupled receptors (GPCRs) engineered with an N-terminal FLAG tag and a C-terminal Venus. The inset images are photomicrographs showing representative cell surface staining (magenta) and Venus fluorescence (green) for a low-expressing receptor (DRD2), a high-expressing receptor (S1 PR1), and a very highly expressed single-transmembrane receptor (EGFR). Background staining was determined by average of signal on untransfected cells and is denoted by the line.

FIG. 18C is a circle chart showing characteristics of the 1791 members of the multi-transmembrane (MTMR) receptor library. Only >500 members have an extracellular HIS tag, and only about half of those receptors show staining above background.

FIG. 18D is a circle chart showing the proportions of the GPCRs of FIG. 18B having low, medium, and high FLAG staining expression levels. “Medium” receptor expression was defined as ten times the background signal.

FIG. 19A is a scatter plot showing results of a screen for binding (a.u.) of EGF-647 to members of the multi-transmembrane receptor library. EGF-647 bound only to EGFR, which was printed on each plate as a transfection control. The inset panel is a photomicrograph showing the fluorescent ligand. DAPI staining is shown.

FIG. 19B is a scatter plot showing results of a screen for binding (a.u.) of RSPO3 to members of the multi-transmembrane receptor library. RSPO3 bound to LGR4 and LGR5. Imaging artifacts are denoted by an X. The inset panel is a photomicrograph showing the fluorescent ligand. DAPI staining is shown.

FIG. 19C is a scatter plot showing results of a screen for binding (a.u.) of PVR to members of the multi-transmembrane receptor library. PVR bound to CD226, a single-pass transmembrane receptor that was added as a positive control. The inset panel is a photomicrograph showing the fluorescent ligand. DAPI staining is shown.

FIG. 19D is a scatter plot showing results of a screen for binding (a.u.) of PD-L1 to members of the multi-transmembrane receptor library. PD-L1 bound to PVR the adhesion G protein-coupled receptor B1 (ADGRB1), as well as to the single-pass transmembrane receptors PD1, PDL2, CD80, and EPHA3, which were added as positive controls. The inset panel is a photomicrograph showing the fluorescent ligand. DAPI staining is shown.

FIG. 20A is a schematic diagram showing an extracellular vesicle (EV) comprising a tagged multi-pass transmembrane receptor. The extracellular regions of the receptor are on the outside of the EV, and the intracellular regions are in the lumen of the EV. Locations of the FLAG tag and fluorescent tag (Luc) are shown.

FIG. 20B is a negative stain electron microscopy image showing EVs.

FIG. 20C is a graph showing the size distribution (in nm) and concentration (10⁶ particles per mL) of EVs from cells transfected with PVR and GAG; transfected with PVR only; or not transfected (control), as measured using NanoSight particle tracking.

FIG. 20D is a bar graph showing the mean size (nm) of the EVs of FIG. 20C.

FIG. 20E is a graph showing the results of a BLI experiment assessing binding of an anti-gD antibody to EVs derived from cells transfected with PVR and GAG; PVR only; or EVs from untransfected cells.

FIG. 20F is a graph showing the results of a BLI experiment assessing binding of the PVR ligand TIGIT (TIGIT Fc) to EVs derived from cells transfected with PVR and GAG; PVR only; or EVs from untransfected cells.

FIG. 20G is a graph showing the results of a BLI experiment assessing binding of an anti-FLAG antibody to EVs comprising the G protein-coupled receptors (GPCRs) ADGRB1, LGR4, and LGR5. GPCRs comprised an N-terminal FLAG tag.

FIG. 21A is a scatter plot showing results of a screen for binding (a.u.) of EVs comprising PVR to members of the multi-transmembrane receptor library and to positive controls. PVR bound to the positive controls. Imaging artifacts are denoted by an X. The inset panel is a photomicrograph showing vesicle fluorescence from GAG-neonGreen fusion. DAPI staining is shown.

FIG. 21B is a scatter plot showing results of a screen for binding (a.u.) of EVs comprising PD-L1 to members of the multi-transmembrane receptor library and to positive controls. PVR bound to the positive controls and to ADGRB1. Imaging artifacts are denoted by an X. The inset panel is a photomicrograph showing vesicle fluorescence from GAG-neonGreen fusion. DAPI staining is shown.

FIG. 21C is a scatter plot showing results of a screen for binding (a.u.) of ADGRB1 to members of a library comprising the extracellular domains of Fc-fused single transmembrane receptors (STMRs). Interactions with RTN4R and PD-L1 were confirmed and new interactions were revealed.

FIG. 22A is a bar graph showing quantification of binding of the recombinant proteins PD-L1-Fc, ICOSLG-Fc, and RTN4R-Fc (each conjugated to a protein A plate) to EVs comprising ADGRB1 or LGR4.

FIG. 22B is a set of photomicrographs showing the results of assays for binding of the recombinant proteins PD-L1-Fc, ICOSLG-Fc, and RTN4R-Fc to HEK cells expressing ADGRB1 fused to Venus. In the merged image, DAPI is shown in blue; the Venus signal from the ADGRB1 fusion protein is shown in green, and the signal for staining of the Fc tag is shown in magenta. Co-localized green and magenta signals are shown in white.

FIG. 23 is a schematic diagram showing the design of the GPCR screening platform. Comprehensive libraries are collected in 384-well plate format. A comprehensive collection of overexpression plasmids are printed onto 384-well imaging plates. Cells are reverse transfected, then treated with fluorescent ligand and analyzed in a high throughput, high content imager FIG. 24A is a set of photomicrographs showing the results of assays for binding of the recombinant protein PD-L1-Fc to HEK cells expressing ADGRB1 fused to Venus. In the merged image, DAPI is shown in blue; the Venus signal from the ADGRB1 fusion protein is shown in green, and the signal for staining of the Fc tag is shown in magenta. Co-localized green and magenta signals are shown in white. All contrast and brightness settings are matched to FIG. 22B.

FIG. 24B is a set of photomicrographs showing the results of assays for binding of the recombinant protein ICOSLG-Fc to HEK cells expressing ADGRB1 fused to Venus. In the merged image, DAPI is shown in blue; the Venus signal from the ADGRB1 fusion protein is shown in green, and the signal for staining of the Fc tag is shown in magenta. Co-localized green and magenta signals are shown in white. All contrast and brightness settings are matched to FIG. 22B.

FIG. 24C is a set of photomicrographs showing the results of assays for binding of the recombinant protein RTN4R-Fc to HEK cells expressing ADGRB1 fused to Venus. In the merged image, DAPI is shown in blue; the Venus signal from the ADGRB1 fusion protein is shown in green, and the signal for staining of the Fc tag is shown in magenta. Co-localized green and magenta signals are shown in white. All contrast and brightness settings are matched to FIG. 22B.

FIG. 25A is a graph showing the results of bioluminescent energy transfer (BRET) assays for β-arrestin and SH2 recruitment in HEK cells. No activation of ADGRB1 or EphA3 was observed in response to PD-L1.

FIG. 25B is a graph showing calcium sensing (GCaMP6s fluorescence) following treatment of HEK cells. No response was observed downstream of ADGRB1 or EphA3 in response to PD-L1.

FIG. 25C is a graph showing cAMP stimulation (assessed by GLOSENSOR™) following treatment of HEK cells. No response was observed.

FIG. 25D is a graph showing cAMP inhibition following treatment of HEK cells. No response was observed.

FIG. 26A is a scatter plot showing results of a screen for binding of EVs comprising ADGRB1 to members of a secreted protein Fc library. A positive control (anti-FLAG antibody) is labeled.

FIG. 26B is a scatter plot showing results of a screen for binding of EVs comprising ADGRB1 to members of a library comprising the extracellular domains of single transmembrane receptors fused to Fc (STM library). Binding to RTN4R and PD-L1 was confirmed, and new interactions were identified. Novel hits are labeled.

FIG. 26C is a scatter plot showing results of a screen for binding of EVs comprising LGR4 to members of a secreted protein Fc library. A positive control (anti-FLAG antibody) is labeled.

FIG. 26D is a scatter plot showing results of a screen for binding of EVs comprising LGR4 to members of the STM library. A positive control (RSPO3) is labeled. Novel hits are shown in green.

FIG. 26E is a scatter plot showing results of a screen for binding of EVs comprising LGR5 to members of a secreted protein Fc library. A positive control (anti-FLAG antibody) is labeled. Novel hits are shown in green.

FIG. 26F is a scatter plot showing results of a screen for binding of EVs comprising LGR5 to members of the STM library. A positive control (anti-FLAG antibody) is labeled. Novel hits are shown in green. Shared LGR4 and LGR5 hits are shown in blue.

FIG. 27 is a bar graph showing the results of a carboxypeptidase M (CPM) activity assay. CPM-FL: vesicles comprised full-length CPM. CPMgD: vesicles comprised gD-GPI (gD) CPM. pRK EV: vector control; PBS only: buffer control.

FIG. 28 is a pair of photomicrographs showing a pair of Western blots showing levels of total EPHA3 and phosphorylated EPHA3 species (pEPHA2/3/4 and pEPHA3/4/5) detected in EVs comprising full-length EPHA3 (EPHA3-FL) and PDL1-Fc, EPHA3-Fc, the EPHA3 ligands EFNA1-Fc and EFNA5-Fc, and full-length PDL1. pRK EV: vector control.

### DETAILED DESCRIPTION OF THE INVENTION

### I. Definitions

As used herein, the term “biological vesicle” or “By” refers to a lipid bilayer-delimited particle that is naturally secreted from a parent cell, e.g., a mammalian cell. BVs may be, e.g., extracellular vesicles (EVs; nanometer-sized particles, e.g., recombinant extracellular vesicles (rEVs)), exosomes, microvesicles, or virus-like particles (VLPs). VLPs are described, e.g., in Titeca et al., Nature Protocols, 12(5): 881-898, 2017). BV compositions or preparations may include only one of EVs, exosomes, microvesicles, or VLPs, or may include a mixture of two, three, or all four of EVs, exosomes, microvesicles, and VLPs. BVs contain proteins folded and inserted into their native membranes using the parent cell's endogenous machinery. In some aspects, BVs include proteins that are not native to the parent cell, e.g., proteins that the parent cell has been modified to express (e.g., heterologous membrane-associated proteins, e.g., heterologous membrane-associated proteins comprising a protein fragment, a tag, and an anchor). Production of BVs by a parent cell may be increased by contacting the parent cell with a membrane-budding agent (e.g., transforming the cell with a membrane-budding agent, e.g., a HIV gag protein) and/or exposing the cell to conditions that promote the formation of BVs. In some aspects, the BV is purified from the parent cell (e.g., purified from a culture medium comprising the parent cell).

As used herein, the term “membrane-budding agent” refers to an agent that increases the production of BVs (e.g., extracellular vesicles (EVs), exosomes, microvesicles, and/or virus-like particles (VLPs)) by the parent cell. In some aspects, the membrane-budding agent is an HIV gag protein. In some aspects, the HIV gag protein has the amino acid sequence of SEQ ID NO: 1. In some aspects, the HIV gag protein has at least 90% identity to the amino acid sequence of SEQ ID NO: 1, e.g., 91%, 92%, 93%, 94%, 95%, 96%, 97%, 98%, or 99% identity to SEQ ID NO: 1. In some aspects, the parent cells are transformed with the membrane-budding agent. Cells may additionally or alternatively be exposed to a condition (e.g., a culture condition) that increases the production of BVs (e.g., extracellular vesicles (EVs), exosomes, microvesicles, and/or virus-like particles (VLPs)) by the parent cell. Further examples of membrane-budding agents include self-assembling VLPs (e.g., MLGag, AARDC1 (e.g., hAARDC1), and Acyl.Hrs); agents that enhance endogenous vesicle formation pathways such as exosome or tumor pathways (e.g., RhoA.F3OL, ARF6.Q67L, VPS4a, HAS3, CD9, CD63, and CD81); and factors associated with apoptotic bodies (e.g., constitutively active ROCK1).

The term “about” as used herein refers to the usual error range for the respective value readily known to the skilled person in this technical field. Reference to “about” a value or parameter herein includes (and describes) aspects that are directed to that value or parameter per se.

The term “single transmembrane receptor,” “single-pass transmembrane receptor,” or “STM receptor,” as used herein, refers to a protein having a single transmembrane domain. In some aspects, the STM receptor is expressed on the cell surface. Exemplary STM receptors are provided in Table 4, as well as in PCT/US2020/025471, Martinez-Martin et al., Cell, 174(5): 1158-1171, 2018, and Clark et al., Genome Res, 13: 2265-2270, 2003, each of which is incorporated by reference herein in its entirety. In some aspects, the STM protein has the UniProt annotation “leucine-rich,” “cysteine-rich,” “ITIM/ITAM” (immunoreceptor tyrosine-based inhibition motif/immunoreceptor tyrosine-based activation motif), “TNFR” (tumor necrosis factor receptor), “TLR/ILR” (Toll-like receptor/interleukin receptor), “semaphorin,” “Kinase-like,” “Ig-like” (immunoglobulin-like), “fibronectin,” “ephrin,” “EGF,” “cytokineR,” or “cadherin.” STM receptors may be identified based on, e.g., the presence of a signal peptide or a predicted transmembrane region in the amino acid sequence. In some aspects, the STM receptor is expressed as an extracellular domain.

As used herein, the term “extracellular domain” or “ECD” refers to a protein domain that is predicted to be localized outside of the outer plasma membrane of the cell. In some instances, the ECD is an ECD of a receptor, e.g., a STM receptor. In some aspects, the ECD is an ECD of an IgSF protein. In some aspects, the ECD is the ECD of PDPN. In some aspects, the boundaries of the extracellular domain may be identified by prediction of domains that indicate that the protein crosses the plasma membrane, e.g., a transmembrane domain (e.g., a transmembrane helix). In some aspects, the presence of an extracellular domain may be predicted by the presence of a domain, sequence, or motif that indicates that the protein is trafficked to the plasma membrane, e.g., a signal sequence or a glycosylphosphatidylinositol (GPI) linkage site. In some aspects, the boundaries of the ECD are determined according to UniProt annotations. In some aspects, the ECD is soluble. In some aspects, the extracellular domain is expressed in the context of a full-length protein. In other aspects, the extracellular domain is expressed as an isolated extracellular domain, e.g., a sequence of amino acid residues comprising only the amino acid residues of a protein that are predicted to be extracellular.

In some aspects, the isolated ECD is included in a fusion protein. In some aspects, inclusion in a fusion protein increases solubility, ease of expression, ease of capture (e.g., on a protein A-coated plate), multimerization, or some other desirable property of the ECD. In some aspects, the ECD or ECD fusion protein is a monomer. In other aspects, the ECD or ECD fusion protein is a multimer, e.g., a tetramer or a pentamer. In some aspects, the ECD is fused to a human IgG. In some aspects, the ECD is fused to a human Fc tag. In some aspects, the ECD is fused to an Avidity AVITAG™ (Avi tag). In some aspects, the ECD is fused to a polyhistidine (His) tag. In some aspects, the ECD is fused to a glycoprotein D (gD) tag and a glycosylphosphatidylinositol (GPI) linker, e.g., a gD-GPI tag. In other aspects, the ECD is fused to the pentamerization domain of rat cartilaginous oligomeric matrix protein (COMP) and the β-lactamase protein, e.g., as described in Bushell et al., Genome Res, 18: 622-630, 2008. In some aspects, the ECD fusion protein further includes a cleavage sequence, e.g., a TEV cleavage sequence, to allow removal of one or more domains. In some instances, an ECD fusion protein having an Avi tag and an Fc tag cleavable at a cleavage sequence is further processed to remove the Fc tag, to biotinylate the Avi tag, and to fuse the biotinylated ECD fusion protein to a fluorescent streptavidin (SA), e.g., to form a tetramerized ECD fusion protein. In some instances, the isolated ECD or ECD fusion protein is purified.

As used herein, a “modulator” is an agent that modulates (e.g., increases, decreases, activates, or inhibits) a given biological activity, e.g., an interaction or a downstream activity resulting from an interaction. A modulator or candidate modulator may be, e.g., a small molecule, an antibody, an antigen-binding fragment (e.g., a bis-Fab, an Fv, a Fab, a Fab′-SH, a F(ab′)₂, a diabody, a linear antibody, an scFv, an ScFab, a VH domain, or a VHH domain), a peptide, a mimic, an antisense oligonucleotide (ASO), or a small interfering RNA (siRNA).

By “increase” or “activate” is meant the ability to cause an overall increase, for example, of 20% or greater, of 50% or greater, or of 75%, 85%, 90%, or 95% or greater. In certain aspects, increase or activate can refer to a downstream activity of a protein-protein interaction.

By “reduce” or “inhibit” is meant the ability to cause an overall decrease, for example, of 20% or greater, of 50% or greater, or of 75%, 85%, 90%, or 95% or greater. In certain aspects, reduce or inhibit can refer to a downstream activity of a protein-protein interaction.

“Affinity” refers to the strength of the sum total of noncovalent interactions between a single binding site of a molecule (e.g., a receptor) and its binding partner (e.g., a ligand). Unless indicated otherwise, as used herein, “binding affinity” refers to intrinsic binding affinity, which reflects a 1:1 interaction between members of a binding pair (e.g., receptor and ligand). The affinity of a molecule X for its partner Y can generally be represented by the dissociation constant (K_{D}). Affinity can be measured by common methods known in the art, including those described herein.

“Complex” or “complexed” as used herein refers to the association of two or more molecules that interact with each other through bonds and/or forces (e.g., Van der Waals, hydrophobic, hydrophilic forces) that are not peptide bonds. In one aspect, a complex is heteromultimeric. It should be understood that the term “protein complex” or “polypeptide complex” as used herein includes complexes that have a non-protein entity conjugated to a protein in the protein complex (e.g., including, but not limited to, chemical molecules such as a toxin or a detection agent).

The term “parent cell” refers to cells from which BVs are produced. Parent cells include cells into which an exogenous nucleic acid has been introduced, including the progeny of such cells. Parent cells include “transfected cells,” “transformed cells,” and “transformants,” which include the primary transformed cell and progeny derived therefrom without regard to the number of passages. Progeny may not be completely identical in nucleic acid content to a parent cell, but may contain mutations. Mutant progeny that have the same function or biological activity as screened or selected for in the originally transformed cell are included herein. In some aspects, the parent cell is stably transformed with the exogenous nucleic acid. In other aspects, the parent cell is transiently transformed with the exogenous nucleic acid.

The term “leucine-rich repeat-containing protein 15” or “LRRC15,” as used herein, broadly refers to any native LRRC15 from any mammalian source, including primates (e.g., humans) and rodents (e.g., mice and rats), unless otherwise indicated. The term encompasses full-length LRRC15 and isolated regions or domains of LRRC15, e.g., the LRRC15 ECD. The term also encompasses naturally occurring variants of LRRC15, e.g., splice variants or allelic variants. The amino acid sequence of an exemplary human LRRC15 is shown under UniProt Accession No. Q8TF66. Minor sequence variations, especially conservative amino acid substitutions of LRRC15 that do not affect LRRC15 function and/or activity, are also contemplated by the invention.

The term “programmed cell death 1 ligand 1” or “PD-L1,” as used herein, broadly refers to any native PD-L1 from any mammalian source, including primates (e.g., humans) and rodents (e.g., mice and rats), unless otherwise indicated. PD-L1 is also called CD274. The term encompasses full-length PD-L1 and isolated regions or domains of PD-L1, e.g., the PD-L1 ECD. The term also encompasses naturally occurring variants of PD-L1, e.g., splice variants or allelic variants. The amino acid sequence of an exemplary human PD-L1 is shown under UniProt Accession No. Q9NZQ7. Minor sequence variations, especially conservative amino acid substitutions of PD-L1 that do not affect PD-L1 function and/or activity, are also contemplated by the invention.

The term “poliovirus receptor” or “PVR,” as used herein, broadly refers to any native PVR from any mammalian source, including primates (e.g., humans) and rodents (e.g., mice and rats), unless otherwise indicated. The term encompasses full-length PVR and isolated regions or domains of PVR, e.g., the PVR ECD. The term also encompasses naturally occurring variants of PVR, e.g., splice variants or allelic variants. The amino acid sequence of an exemplary human PVR is shown under UniProt Accession No. A0A0C4DG49. Minor sequence variations, especially conservative amino acid substitutions of PVR that do not affect PVR function and/or activity, are also contemplated by the invention.

The term “CD80,” as used herein, broadly refers to any native CD80 from any mammalian source, including primates (e.g., humans) and rodents (e.g., mice and rats), unless otherwise indicated. CD80 is also called B7-1. The term encompasses full-length CD80 and isolated regions or domains of CD80, e.g., the CD80 ECD. The term also encompasses naturally occurring variants of CD80, e.g., splice variants or allelic variants. The amino acid sequence of an exemplary human CD80 is shown under UniProt Accession No. P33681. Minor sequence variations, especially conservative amino acid substitutions of CD80 that do not affect CD80 function and/or activity, are also contemplated by the invention.

The term “CD276,” as used herein, broadly refers to any native CD276 from any mammalian source, including primates (e.g., humans) and rodents (e.g., mice and rats), unless otherwise indicated. CD276 is also called B7-H3. The term encompasses full-length CD276 and isolated regions or domains of CD276, e.g., the CD276 ECD. The term also encompasses naturally occurring variants of CD276, e.g., splice variants or allelic variants. The amino acid sequence of an exemplary human CD276 is shown under UniProt Accession No. Q5ZPR3. Minor sequence variations, especially conservative amino acid substitutions of CD276 that do not affect CD276 function and/or activity, are also contemplated by the invention.

The terms “TEM1,” “CD248,” and “endosialin,” as used herein, broadly refer to any native TEM1 from any mammalian source, including primates (e.g., humans) and rodents (e.g., mice and rats), unless otherwise indicated. The term encompasses full-length TEM1 and isolated regions or domains of TEM1, e.g., the TEM1 ECD. The term also encompasses naturally occurring variants of TEM1, e.g., splice variants or allelic variants. The amino acid sequence of an exemplary human TEM1 is shown under UniProt Accession No. Q9HCU0. Minor sequence variations, especially conservative amino acid substitutions of TEM1 that do not affect TEM1 function and/or activity, are also contemplated by the invention.

The terms “ADGRB1,” “adhesion GPCR B11,” and “adhesion G protein-coupled receptor B11,” as used herein, broadly refer to any native ADGRB1 from any mammalian source, including primates (e.g., humans) and rodents (e.g., mice and rats), unless otherwise indicated. The term encompasses full-length ADGRB1 and isolated regions or domains of ADGRB1, e.g., the ADGRB1 ECDs. The term also encompasses naturally occurring variants of ADGRB1, e.g., splice variants or allelic variants. The amino acid sequence of an exemplary human ADGRB1 is shown under UniProt Accession No. O14514. Minor sequence variations, especially conservative amino acid substitutions of TEM1 that do not affect TEM1 function and/or activity, are also contemplated by the invention.

The terms “ICOSLG,” “inducible T cell costimulatory ligand,” and “ICOS ligand,” as used herein, broadly refer to any native ICOSLG from any mammalian source, including primates (e.g., humans) and rodents (e.g., mice and rats), unless otherwise indicated. The term encompasses full-length ICOSLG and isolated regions or domains of ICOSLG, e.g., the ICOSLG ECD. The term also encompasses naturally occurring variants of ICOSLG, e.g., splice variants or allelic variants. The amino acid sequence of an exemplary human ICOSLG is shown under UniProt Accession No. 075144. Minor sequence variations, especially conservative amino acid substitutions of ICOSLG that do not affect ICOSLG function and/or activity, are also contemplated by the invention.

The term “protein,” as used herein, refers to any native protein from any vertebrate source, including mammals such as primates (e.g., humans) and rodents (e.g., mice and rats), unless otherwise indicated. The term encompasses “full-length,” unprocessed protein any form of the protein that results from processing in the cell. The term also encompasses naturally occurring variants of the protein, e.g., splice variants or allelic variants, e.g., amino acid substitution mutations or amino acid deletion mutations. The term also includes isolated regions or domains of the protein, e.g., the extracellular domain (ECD).

An “isolated” protein or peptide is one which has been separated from a component of its natural environment. In some aspects, a protein or peptide is purified to greater than 95% or 99% purity as determined by, for example, electrophoresis (e.g., SDS-PAGE, isoelectric focusing (IEF), capillary electrophoresis) or chromatography (e.g., ion exchange or reverse phase HPLC).

An “isolated” nucleic acid refers to a nucleic acid molecule that has been separated from a component of its natural environment. An isolated nucleic acid includes a nucleic acid molecule contained in cells that ordinarily contain the nucleic acid molecule, but the nucleic acid molecule is present extrachromosomally or at a chromosomal location that is different from its natural chromosomal location.

The term “vector,” as used herein, refers to a nucleic acid molecule capable of propagating another nucleic acid to which it is linked. The term includes the vector as a self-replicating nucleic acid structure as well as the vector incorporated into the genome of a host cell into which it has been introduced. Certain vectors are capable of directing the expression of nucleic acids to which they are operatively linked. Such vectors are referred to herein as “expression vectors.”

The term “antibody” herein is used in the broadest sense and encompasses various antibody structures, including but not limited to monoclonal antibodies, polyclonal antibodies, multispecific antibodies (e.g., bispecific antibodies), and antibody fragments (e.g., bis-Fabs) so long as they exhibit the desired antigen-binding activity.

An “antigen-binding fragment” or “antibody fragment” refers to a molecule other than an intact antibody that comprises a portion of an intact antibody that binds the antigen to which the intact antibody binds. Examples of antigen-binding fragments include but are not limited to bis-Fabs; Fv; Fab; Fab, Fab′-SH; F(ab′)₂; diabodies; linear antibodies; single-chain antibody molecules (e.g., scFv, ScFab); and multispecific antibodies formed from antibody fragments.

A “single-domain antibody” refers to an antibody fragment comprising all or a portion of the heavy chain variable domain or all or a portion of the light chain variable domain of an antibody. In certain aspects, a single-domain antibody is a human single-domain antibody (see, e.g., U.S. Pat. No. 6,248,516 B1). Examples of single-domain antibodies include but are not limited to a VHH.

A “Fab” fragment is an antigen-binding fragment generated by papain digestion of antibodies and consists of an entire L chain along with the variable region domain of the H chain (VH), and the first constant domain of one heavy chain (CH1). Papain digestion of antibodies produces two identical Fab fragments. Pepsin treatment of an antibody yields a single large F(ab′)₂ fragment which roughly corresponds to two disulfide linked Fab fragments having divalent antigen-binding activity and is still capable of cross-linking antigen. Fab′ fragments differ from Fab fragments by having an additional few residues at the carboxy terminus of the CH1 domain including one or more cysteines from the antibody hinge region. Fab′-SH is the designation herein for Fab′ in which the cysteine residue(s) of the constant domains bear a free thiol group. F(ab′)₂ antibody fragments originally were produced as pairs of Fab′ fragments which have hinge cysteines between them. Other chemical couplings of antibody fragments are also known.

The term “Fc region” herein is used to define a C-terminal region of an immunoglobulin heavy chain, including native sequence Fc regions and variant Fc regions. Although the boundaries of the Fc region of an immunoglobulin heavy chain might vary, the human IgG heavy chain Fc region is usually defined to stretch from an amino acid residue at position Cys226, or from Pro230, to the carboxyl-terminus thereof. The C-terminal lysine (residue 447 according to the EU numbering system) of the Fc region may be removed, for example, during production or purification of the antibody, or by recombinantly engineering the nucleic acid encoding a heavy chain of the antibody. Accordingly, a composition of intact antibodies may comprise antibody populations with all Lys447 residues removed, antibody populations with no Lys447 residues removed, and antibody populations having a mixture of antibodies with and without the Lys447 residue.

“Fv” consists of a dimer of one heavy- and one light-chain variable region domain in tight, noncovalent association. From the folding of these two domains emanate six hypervariable loops (3 loops each from the H and L chain) that contribute the amino acid residues for antigen binding and confer antigen binding specificity to the antibody. However, even a single variable domain (or half of an Fv comprising only three CDRs specific for an antigen) has the ability to recognize and bind antigen, although often at a lower affinity than the entire binding site.

The terms “full-length antibody,” “intact antibody,” and “whole antibody” are used herein interchangeably to refer to an antibody having a structure substantially similar to a native antibody structure or having heavy chains that contain an Fc region as defined herein.

“Single-chain Fv” also abbreviated as “sFv” or “scFv” are antibody fragments that comprise the VH and VL antibody domains connected into a single polypeptide chain. Preferably, the scFv polypeptide further comprises a polypeptide linker between the VH and VL domains, which enables the scFv to form the desired structure for antigen binding. For a review of scFv, see Pluckthun, The Pharmacology of Monoclonal Antibodies, vol. 113, Rosenburg and Moore eds., Springer-Verlag, New York, pp. 269-315 (1994); Malmborg et al., J. Immunol. Methods 183:7-13, 1995.

The term “small molecule” refers to any molecule with a molecular weight of about 2000 daltons or less, e.g., about 1000 daltons or less. In some aspects, the small molecule is a small organic molecule.

The term “mimic,” “peptide mimic,” “polypeptide mimic,” or “molecular mimic,” as used herein, refers to a polypeptide having sufficient similarity in conformation and/or binding ability (e.g., secondary structure, tertiary structure) to a given polypeptide or to a portion of said polypeptide to bind to a binding partner of said polypeptide. The mimic may bind the binding partner with equal, less, or greater affinity than the polypeptide it mimics. A molecular mimic may or may not have obvious amino acid sequence similarity to the polypeptide it mimics. A mimic may be naturally occurring or may be engineered. In some aspects, the mimic is a mimic of the protein of Table 1. In other aspects, the mimic is a mimic of the protein of Table 2. In yet other aspects, the mimic is a mimic of another protein that binds to the protein of Table 1 or the protein of Table 2. In some aspects, the mimic is a mimic of the protein of Table 5. In other aspects, the mimic is a mimic of the protein of Table 6. In yet other aspects, the mimic is a mimic of another protein that binds to the protein of Table 5 or the protein of Table 6. In some aspects, the mimic may perform all functions of the mimicked polypeptide. In other aspects, the mimic does not perform all functions of the mimicked polypeptide.

As used herein, the term “conditions permitting the binding” of two or more proteins to each other (e.g., a protein of Table 1 and a protein of Table 2 or a protein of Table 5 and a protein of Table 6) refers to conditions (e.g., protein concentration, temperature, pH, salt concentration) under which the two or more proteins would interact in the absence of a modulator or a candidate modulator. Conditions permitting binding may differ for individual proteins and may differ between protein-protein interaction assays (e.g., surface plasmon resonance assays, biolayer interferometry assays, enzyme-linked immunosorbent assays (ELISA), extracellular interaction assays, and cell surface interaction assays.

“Percent (%) amino acid sequence identity” with respect to a reference polypeptide sequence is defined as the percentage of amino acid residues in a candidate sequence that are identical with the amino acid residues in the reference polypeptide sequence, after aligning the sequences and introducing gaps, if necessary, to achieve the maximum percent sequence identity, and not considering any conservative substitutions as part of the sequence identity. Alignment for purposes of determining percent amino acid sequence identity can be achieved in various ways that are within the skill in the art, for instance, using publicly available computer software such as BLAST, BLAST-2, ALIGN or Megalign (DNASTAR) software. Those skilled in the art can determine appropriate parameters for aligning sequences, including any algorithms needed to achieve maximal alignment over the full-length of the sequences being compared. For purposes herein, however, % amino acid sequence identity values are generated using the sequence comparison computer program ALIGN-2. The ALIGN-2 sequence comparison computer program was authored by Genentech, Inc., and the source code has been filed with user documentation in the U.S. Copyright Office, Washington D.C., 20559, where it is registered under U.S. Copyright Registration No. TXU510087. The ALIGN-2 program is publicly available from Genentech, Inc., South San Francisco, California, or may be compiled from the source code. The ALIGN-2 program should be compiled for use on a UNIX operating system, including digital UNIX V4.0D. All sequence comparison parameters are set by the ALIGN-2 program and do not vary.

In situations where ALIGN-2 is employed for amino acid sequence comparisons, the % amino acid sequence identity of a given amino acid sequence A to, with, or against a given amino acid sequence B (which can alternatively be phrased as a given amino acid sequence A that has or comprises a certain % amino acid sequence identity to, with, or against a given amino acid sequence B) is calculated as follows:

100 times the fraction X/Y

where X is the number of amino acid residues scored as identical matches by the sequence alignment program ALIGN-2 in that program's alignment of A and B, and where Y is the total number of amino acid residues in B. It will be appreciated that where the length of amino acid sequence A is not equal to the length of amino acid sequence B, the % amino acid sequence identity of A to B will not equal the % amino acid sequence identity of B to A. Unless specifically stated otherwise, all % amino acid sequence identity values used herein are obtained as described in the immediately preceding paragraph using the ALIGN-2 computer program.

The term “sample,” as used herein, refers to a composition that is obtained or derived from a subject and/or individual of interest that contains a cellular and/or other molecular entity that is to be characterized and/or identified, for example, based on physical, biochemical, chemical, and/or physiological characteristics. For example, the phrase “disease sample” and variations thereof refers to any sample obtained from a subject of interest that would be expected or is known to contain the cellular and/or molecular entity that is to be characterized. Samples include, but are not limited to, tissue samples, primary or cultured cells or cell lines, cell supernatants, cell lysates, platelets, serum, plasma, vitreous fluid, lymph fluid, synovial fluid, follicular fluid, seminal fluid, amniotic fluid, milk, whole blood, plasma, serum, blood-derived cells, urine, cerebro-spinal fluid, saliva, buccal swab, sputum, tears, perspiration, mucus, tumor lysates, and tissue culture medium, tissue extracts such as homogenized tissue, tumor tissue, cellular extracts, and combinations thereof. The sample may be an archival sample, a fresh sample, or a frozen sample. In some aspects, the sample is a formalin-fixed and paraffin-embedded (FFPE) tumor tissue sample.

### II. Biological Vesicles Displaying Proteins

The study of interactions involving membrane-bound proteins (e.g., receptor-ligand interactions) is instrumental to understanding cellular communication occurring in the extracellular milieu. However, progress on identifying and understanding these interactions has lagged behind that of cytoplasmic proteins, in part because receptor-ligand interactions take place in membranes. Physiological membranes contain a complex mix of lipids, sterols, proteins and glycans, all of which can participate in interactions. In addition, membranes help cluster, orient and fold receptors, strengthening weak protein-protein interactions. Standard methods for assessing protein-protein interactions typically require the absence of, or extraction from, cellular membranes. As a result, these commonly utilized methodologies underrepresent receptor-ligand interactions.

The disclosure features proteins (e.g., transmembrane receptors) displayed on the surface of biological vesicles (BVs), e.g., extracellular vesicles (EVs). In some aspects, the disclosure features a BV comprising (a) a heterologous membrane-associated protein comprising a protein fragment, a tag, and an anchor, wherein the heterologous membrane-associated protein is present on the outer face of the BV and (b) a membrane-budding agent. In some aspects, the membrane-budding agent is an HIV gag protein.

In some aspects, the disclosure features a BV comprising (a) a heterologous membrane-associated protein comprising a protein fragment, a tag, and an anchor, wherein the heterologous membrane-associated protein is present on the outer face of the BV and (b) a membrane-budding agent, wherein the membrane budding agent is an HIV gag protein, the BV being produced by a process comprising (i) providing a parent cell that has been modified to express the heterologous membrane-associated protein and the membrane-budding agent and (ii) isolating the BV from the parent cell.

A. Protein Fragments

In some aspects, a protein fragment is an extracellular domain of a transmembrane receptor, e.g., a single-pass transmembrane (STM) receptor or a multi-pass transmembrane receptor (multi-transmembrane receptor; MTMR), e.g., a G protein-coupled receptor (GPCR). Exemplary STM receptors are described in Section III herein and are provided in Table 2 and Table 4.

B. Anchors

In some aspects, an anchor tethers the protein fragment to the surface of a lipid membrane of a BV. In some aspects, the anchor is a glycosylphosphatidyl-inositol (GPI) polypeptide. In some aspects, the anchor is a moiety used in protein lipidation, e.g., a moiety used in cysteine palmitoylation, glycine myristoylation, lysine fatty-acylation, cholesterol esterification, cysteine prenylation, or serine fatty-acylation.

C. Tags

In some aspects, a tag can be directly or indirectly visualized, or otherwise detected. For example, the tag may comprise a moiety that can be detected using an antibody or an antibody fragment, e.g., may be a glycoprotein D (gD) polypeptide. In some aspects, the tag comprises a fluorescent protein. In some aspects, the protein fragment is conjugated (e.g., fused) to a gD-GPI construct comprising a gD tag and a GPI anchor.

D. Membrane-Budding Agents

BVs may further comprise a membrane-budding agent that, when present in the parent cell, increases the production of BVs (e.g., extracellular vesicles (EVs), exosomes, microvesicles, and/or virus-like particles (VLPs)) by the parent cell. The parent cell may be transfected with the membrane-budding agent, and the membrane-budding agent may be inherited by the BV, e.g., during the membrane budding process.

In some aspects, the membrane-budding agent is an HIV gag protein. In some aspects, the HIV gag protein has the amino acid sequence of SEQ ID NO: 1. In some aspects, the HIV gag protein has at least 90% identity to the amino acid sequence of SEQ ID NO: 1, e.g., 91%, 92%, 93%, 94%, 95%, 96%, 97%, 98%, or 99% identity to SEQ ID NO: 1. In some aspects, the membrane-budding agent (e.g., HIV gag protein) comprises a marker that can be directly or indirectly visualized, or otherwise detected. In some aspects, the detectable marker is a fluorescent protein. In some aspects, the detectable marker is an enzyme that produces a fluorescent signal in the presence of a substrate, e.g., Renilla luciferase (Rluc). Additional membrane-budding agents are described in Section III(C), below.

Cells may additionally or alternatively be exposed to a condition (e.g., a culture condition) that increases the production of BVs (e.g., extracellular vesicles (EVs), exosomes, microvesicles, and/or virus-like particles (VLPs)) by the parent cell. EVs comprising a membrane-budding agent may be referred to as recombinant EVs (rEVs).

The membrane-budding agent and/or agent or condition that increases BV production may increase BV production by the parent cell by, e.g., 1.5-fold, 2-fold, 2.5-fold, 3-fold, 3.5-fold, 4-fold, or more than 4-fold (e.g., 1.5 to 2.5-fold, 2.5 to 3.5-fold, or 3.5 to 4.5-fold). In some aspects, the membrane-budding agent (e.g., HIV gag protein) increases BV production by the parent cell by about 4-fold.

E. Parent Cells and Methods of Isolation

Biological vesicles (BVs) include any suitable lipid vesicle structure that has been derived from (e.g., produced by and separated from) a parent cell. In some aspects, the BV is produced by a mammalian cell. The cell may be, e.g., an EXPI293F™ cell. In some aspects, the BV is an extracellular vesicle (EV), an exosome, a microvesicle, or a virus-like particle (VLP). In some aspects, BV preparations or compositions include mixtures of EVs, exosomes, microvesicles, and/or VLPs. BVs may be separated from parent cells and/or large EVs and protein aggregates (e.g., separated from the culture media of parent cells), e.g., using centrifugation (e.g., ultracentrifugation).

In some aspects, the parent cell has been transfected with a plasmid encoding the heterologous membrane-associated protein and a plasmid encoding the membrane-budding agent. The heterologous membrane-associated protein and the membrane-budding agent may be encoded by a single plasmid, or may be encoded by separate plasmids.

The level of expression of the heterologous membrane-associated protein and/or the membrane-budding agent on the surface of the parent cell and/or on the surface of the BV may be assessed. In some aspects, the level of expression of the heterologous membrane-associated protein is assessed using biolayer interferometry (BLI), wherein the BV produces at least a shift that is at or above a threshold level when contacted with an antibody against the tag associated with the heterologous membrane-associated protein. In some aspects, the tag is a gD polypeptide, the antibody is an anti-gD antibody, and the shift is at least 1.5 nm when the BLI assay is performed at 30° C. In other aspects, the BV is contacted with an antibody specific for the heterologous membrane-associated protein.

In some aspects, the BV comprises a marker that allows direct or indirect visualization of the BV, e.g., a membrane marker (e.g., a fluorescent membrane marker). In some aspects, the membrane marker is a cholesterol marker, e.g., AMPLEX™ Red.

### III. Methods of Identifying Protein-Protein Interactions

Biological vesicles (BVs) provide a protein-purification free method for obtaining binding-competent receptors. The BVs bearing receptors may then be tested for interaction with ligands of the receptor (e.g., libraries of ligands), thus providing a method for identifying and assessing protein-protein interactions.

In some aspects, the disclosure features a method for identifying a protein-protein interaction, the method comprising (a) providing a collection of target polypeptides, optionally wherein the collection of target polypeptides is immobilized on one or more solid surfaces; (b) contacting the collection of step (a) with a biological vesicle (BV) comprising a heterologous membrane-associated protein and a membrane-budding agent under conditions permitting the binding of the heterologous membrane-associated protein and at least one of the target polypeptides, wherein the membrane budding agent is an HIV gag protein and wherein the heterologous membrane-associated protein is expressed at or above a threshold level on the surface of the BV; and (c) detecting an interaction between the heterologous membrane-associated protein and the at least one target polypeptide, thereby identifying a protein-protein interaction.

A. Heterologous Membrane-Associated Proteins

The heterologous membrane-associated protein may be any protein or polypeptide or fragment thereof that can be incorporated into an EV.

In some aspects, the heterologous membrane-associated protein is a full-length protein. In other aspects, the heterologous membrane-associated protein comprises a protein fragment, a tag, and an anchor. The protein fragment may be, e.g., an extracellular domain (e.g., an extracellular domain of a protein of interest, e.g., a transmembrane receptor. An ECD is a domain of a protein that is predicted to be localized outside of the plasma membrane of the cell. This domain of the protein is thus available to interact with the extracellular environment, e.g., interact with soluble proteins and ECDs of other proteins on the cell or on an adjacent cell. The ECD or ECDs of a protein may be identified by bioinformatics analysis, e.g., by analysis of UniProt annotations. For example, the boundaries of the ECD may be identified relative to the boundary of an adjacent predicted transmembrane region, e.g., a transmembrane helix. In some aspects, the presence of an extracellular domain may be predicted by the presence of a domain, sequence, or motif that indicates that the protein is trafficked to the plasma membrane, e.g., a signal sequence or a glycosylphosphatidylinositol (GPI) linkage site. In some aspects, the extracellular domain is expressed in the context of a full-length protein. In other aspects, the extracellular domain is expressed as an isolated extracellular domain, e.g., a sequence of amino acid residues comprising only the amino acid residues of a protein that are predicted to be extracellular. In some aspects, the isolated extracellular domain is expressed in a fusion protein.

In some aspects in which the heterologous membrane-associated protein comprises a protein fragment, a tag, and an anchor, the anchor tethers the protein fragment to the surface of a lipid membrane of a BV. In some aspects, the anchor is a glycosylphosphatidyl-inositol (GPI) polypeptide. In some aspects, the anchor is a moiety used in protein lipidation, e.g., a moiety used in cysteine palmitoylation, glycine myristoylation, lysine fatty-acylation, cholesterol esterification, cysteine prenylation, or serine fatty-acylation.

In some aspects in which the heterologous membrane-associated protein comprises a protein fragment, a tag, and an anchor, the tag can be directly or indirectly visualized, or otherwise detected. For example, the tag may comprise a moiety that can be detected using an antibody or an antibody fragment, e.g., may be a glycoprotein D (gD) polypeptide. In some aspects, the tag comprises a fluorescent protein.

In some aspects, the protein fragment is conjugated (e.g., fused) to a gD-GPI construct comprising a gD tag and a GPI anchor. In some aspects, the heterologous membrane-associated protein is a protein provided in Table 1 (e.g., an ECD of a protein provided in Table 1 conjugated to a gD-GPI construct) or a protein provided in Table 5, below.

TABLE 1
BV-expressed proteins

LRRC15
PD-L1/CD274
PVR
CD80/B7-1
CD276/B7-H3

B. BVs and Parent Cells

Biological vesicles (BVs) include any suitable lipid vesicle structure that has been derived from (e.g., produced by and separated from) a parent cell, as described in Section II(E) herein.

In some aspects, the parent cell has been transfected with a plasmid encoding the heterologous membrane-associated protein and a plasmid encoding the membrane-budding agent. The heterologous membrane-associated protein and the membrane-budding agent may be encoded by a single plasmid, or may be encoded by separate plasmids.

The level of expression of the heterologous membrane-associated protein and/or the membrane-budding agent on the surface of the parent cell and/or on the surface of the BV may be assessed. In some aspects, the level of expression of the heterologous membrane-associated protein is assessed using biolayer interferometry (BLI), wherein the BV produces at least a shift that is at or above a threshold level when contacted with an antibody against the tag associated with the heterologous membrane-associated protein. In some aspects, the tag is a gD polypeptide, the antibody is an anti-gD antibody, and the threshold level is a shift of at least 1.5 nm when the BLI assay is performed at 30° C. In other aspects, the BV is contacted with an antibody specific for the heterologous membrane-associated protein.

In some aspects, the BV comprises a marker that allows direct or indirect visualization of the BV, e.g., a membrane marker (e.g., a fluorescent membrane marker). In some aspects, the membrane marker is a cholesterol marker, e.g., AMPLEX™ Red.

C. Membrane-Budding Agents

BVs may further comprise a membrane-budding agent that increases the production of BVs (e.g., extracellular vesicles (EVs), exosomes, microvesicles, and/or virus-like particles (VLPs)) by the parent cell, as described in Section II(D) herein.

In some aspects, the membrane-budding agent is an HIV gag protein. In some aspects, the HIV gag protein has the amino acid sequence of SEQ ID NO: 1. In some aspects, the HIV gag protein has at least 90% identity to the amino acid sequence of SEQ ID NO: 1, e.g., 91%, 92%, 93%, 94%, 95%, 96%, 97%, 98%, or 99% identity to SEQ ID NO: 1.

Further exemplary membrane budding agents include self-assembling VLPs (e.g., MLGag, AARDC1 (e.g., hAARDC1), and Acyl.Hrs); agents that enhance endogenous vesicle formation pathways such as exosome or tumor pathways (e.g., RhoA.F30L, ARF6.Q67L, VPS4a, HAS3, CD9, CD63, and CD81); and factors associated with apoptotic bodies (e.g., constitutively active ROCK1).

In some embodiments, the membrane budding agent is MLGag, Acyl.Hrs, ARRDC1 (e.g., hAARDC1), ARF6 (e.g., ARF6Q67L), RhoA (e.g., RhoA.F30L), or a combination thereof.

In some aspects, the membrane budding agent is a Gag protein, e.g., a chimeric Gag protein (e.g., a chimeric Gag protein as discussed in Hammarstedt et al., J Virol. 78(11): 5686-97, 2004 or Chen et al., Proc Natl Acad Sci USA, 98(26): 15239-44, 2001). In some aspects, the chimeric Gag protein comprises a portion of HIV Gag and a portion of Gag from a different retrovirus. For example, but not by way of limitation, the chimeric Gag comprises an HIV Gag, wherein a region of the HIV Gag known to direct its localization is replaced with functionally homologous regions from Moloney murine leukemia virus (MLV), a murine retrovirus. In certain embodiments, the replaced region of the HIV Gag is a matrix domain (MA), thus generating a chimeric Gag referred to herein as MLGag. In certain embodiments, chimeric and full-length Gag proteins can be generated from endogenous retrovirus (ERV) sequences derived from any species, e.g., as described in Stocking et al., Cell Mol. Life Sci., 65(21):3383-3398, 2008. In certain embodiments, the vesicle factor is MLGag.

In certain embodiments, the vesicle factor is an arrestin domain-containing protein 1 (ARRDC1). In certain embodiments, the vesicle factor is a murine ARRDC1 (mARRDC1). In certain embodiments, the vesicle factor is a human ARRDC1 (hARRDC1). ARRDC1 is a tetrapeptide PSAP motif of an accessory protein and is a host protein that induces EV formation. It has been shown that overexpression of ARRDC 1 results in enhanced microvesicle (MV) formation. Such effect is mediated by the recruitment of Tsg 101 via PSAP/PTAP peptide. Overexpression of ATPase VP S4a results in further enhancement in MV formation (Nabhan et al., Proc Natl Acad Sci USA, 109(11): 4146-51, 2012).

In certain embodiments, the vesicle factor is ADP ribosylation factor-6 (ARF6). It has been shown that ARF6 is a Rho GTPase that drives microvesicle formation in tumor cells in an ERK-dependent manner (Muralidharan-Chari et al., Curr Biol., 19(22): 1875-85, 2009. In certain embodiments, the vesicle factor is a constitutively active form of ARF6. For example, but not by way of limitation, the constitutively active form of ARF6 is ARF6.Q67L (see, e.g., Peters et al., J. Cell Biol, 128(6):1003-1017, 1995).

In certain embodiments, the vesicle factor is a mutant RhoA/ROCK1 that can also drive microvesicle formation in tumor cells (Li et al., Oncogene, 31(45): 4740-9, 2012). In certain embodiments, the vesicle factor is a constitutively active form of RhoA. For example, but not by way of limitation, the constitutively active form of RhoA is RhoA.F30L (see, e.g., Lin et al., JBC, 274(33): 23633-23641, 1999).

In certain embodiments, the vesicle factor comprises a plasma membrane (PM) binding domain, a self-assembly domain, and an endosomal sorting complex required for transport (ESCRT) recruiting domain. The design principle for EV formation is to enable rapid generation of new EV factors/cargo. It has been shown that PM targeting and high order oligomerization drives EV incorporation (Fang et al., PLoS Biol., 5(6): e158, 2007. In certain embodiments, the vesicle factor is Acyl.Hrs that comprises a PM binding domain of acylation tag and the C-terminal domain of hepatocyte growth factor-regulated tyrosine kinase substrate (Hrs) that consists of a self-assembly domain of coiled coils, and an ESCRT recruiting domain. In certain embodiments, the vesicle factor is MLGag that comprises a PM binding domain of Matrix, a self-assembly domain of capsid, and an ESCRT recruiting domain of p6. In certain embodiments, the vesicle factor comprises a self-assembly domain and an ESCRT recruiting domain. In certain embodiments, the vesicle factor is ARRDC1 that comprises a self-assembly domain of arrestin domain, and an ESCRT recruiting domain.

Additional vesicle factors can be identified by any method known in the art. For example, but not by way of limitation, a screen of a cDNA library of all proteins, e.g., human proteins, can be performed to identify a single gene or a combination of genes that increases production of EVs. Alternatively or additionally, a CRISPR or RNAi screen can be performed to identify a single gene or a combination of genes that inhibits production of EVs.

In some aspects, the membrane-budding agent (e.g., HIV gag protein) comprises a marker that can be directly or indirectly visualized, or otherwise detected. In some aspects, the detectable marker is an enzyme that produces a fluorescent signal in the presence of a substrate, e.g., the enzyme is Renilla luciferase (Rluc) and the substrate is Rluc substrate.

Cells may additionally or alternatively be exposed to a condition (e.g., a culture condition) that increases the production of BVs (e.g., extracellular vesicles (EVs), exosomes, microvesicles, and/or virus-like particles (VLPs)) by the parent cell.

The membrane-budding agent and/or agent or condition that increases BV production may increase BV production by the parent cell by, e.g., 1.5-fold, 2-fold, 2.5-fold, 3-fold, 3.5-fold, 4-fold, or more than 4-fold (e.g., 1.5 to 2.5-fold, 2.5 to 3.5-fold, or 3.5 to 4.5-fold). In some aspects, the membrane-budding agent (e.g., HIV gag protein) increases BV production by the parent cell by about 4-fold.

D. Collections of Target Polypeptides

In some aspects, the collection of target polypeptides is a collection of transmembrane receptors or fragments thereof. In some aspects, the receptors are single-pass transmembrane (STM) receptors. STM receptor proteins are a large category of membrane-bound receptors having a single domain passing through the plasma membrane. Many STM receptors are expressed on the cell surface, and thus may participate in the extracellular interactome. Exemplary STM receptors are provided in Tables 2 and 4 and in Martinez-Martin et al., Cell, 174(5): 1158-1171, 2018 and Clark et al., Genome Res, 13: 2265-2270, 2003.

In some aspects, the protein fragment is an extracellular domain (ECD), e.g., an ECD identified as described above. In some aspects, each member of the collection of target polypeptides is an Fc-tagged extracellular domain, and the solid surface is coated with protein A. In some aspects, one or more of the target polypeptides is immobilized to a distinct location on the one or more solid surfaces. In other aspects, the one or more target polypeptides are not immobilized to a surface.

TABLE 2
STM library proteins

TEM1/CD248/endosialin
PLXDC2
PTPRD
SARAF
ASGR1
BMP10
CPM
LDLR
PILRA
PRRG2
C6orf72
LRTM1
CDHR2
IGF2R
NCR3
SUSD3
CLEC17A
PVRL4
BTNL3
CDHR2
GLT8D2
KIAA1467
RNF152
LRFN1
MXRA5
PVRL1
LRIT2
PLA2R1
SLITRK4

TABLE 3
Interactions between BV-expressed
proteins and STM library proteins

| Partner A | Partner B |
|---|---|
| LRRC15 | TEM1/CD248/endosialin |
| | PLXDC2 |
| | PTPRD |
| | SARAF |
| | ASGR1 |
| | BMP10 |
| | CPM |
| | LDLR |
| | PILRA |
| | PRRG2 |
| PD-L1/CD274 | C6orf72 |
| | LRTM1 |
| | CDHR2 |
| | IGF2R |
| | NCR3 |
| | SUSD3 |
| PVR | CLEC17A |
| | PVRL4 |
| CD80/B7-1 | BTNL3 |
| | CDHR2 |
| | GLT8D2 |
| | KIAA1467 |
| | RNF152 |
| CD276/B7-H3 | LRFN1 |
| | MXRA5 |
| | PVRL1 |
| | LRIT2 |
| | PLA2R1 |
| | SLITRK4 |

TABLE 4
Tagged STM library

| Prey short name | Prey Entrez ID |
|---|---|
| 1110032F04RIK | 68725 |
| A1BG | 1 |
| ACE | 1636 |
| ACE2 | 59272 |
| ACPP | 55 |
| ACPT | 93650 |
| ACVR1 | 90 |
| ACVR1B | 91 |
| ACVR1C | 130399 |
| ACVR2A | 92 |
| ACVR2B | 93 |
| ACVRL1 | 94 |
| ADAM10 | 102 |
| ADAM11 | 4185 |
| ADAM12 | 8038 |
| ADAM15 | 8751 |
| ADAM17 | 6868 |
| ADAM18 | 8749 |
| ADAM19 | 8728 |
| ADAM2 | 2515 |
| ADAM20 | 8748 |
| ADAM21 | 8747 |
| ADAM22 | 53616 |
| ADAM23 | 8745 |
| ADAM28 | 10863 |
| ADAM29 | 11086 |
| ADAM30 | 11085 |
| ADAM32 | 203102 |
| ADAM33 | 80332 |
| ADAM7 | 8756 |
| ADAM8 | 101 |
| ADAM9 | 8754 |
| AGER | 177 |
| AJAP1 | 55966 |
| ALCAM | 214 |
| ALK | 238 |
| ALPI | 248 |
| ALPL | 249 |
| ALPP | 250 |
| AMHR2 | 269 |
| AMICA1 | 120425 |
| AMIGO1 | 57463 |
| AMIGO2 | 347902 |
| AMIGO3 | 386724 |
| AMN | 81693 |
| ANTXR1 | 84168 |
| ANTXR2 | 118429 |
| APCDD1 | 147495 |
| APCDD1L | 164284 |
| APLP1 | 333 |
| APLP2 | 334 |
| APOO | 79135 |
| APP | 351 |
| AREG | 374 |
| ART1 | 417 |
| ART3 | 419 |
| ART4 | 420 |
| ATRAID | 51374 |
| ATRN | 8455 |
| ATRNL1 | 26033 |
| AXL | 558 |
| AZGP1 | 563 |
| BACE1 | 23621 |
| BACE2 | 25825 |
| BAMBI | 25805 |
| BCAM | 4059 |
| BCAN | 63827 |
| BMPR1A | 657 |
| BMPR1B | 658 |
| BMPR2 | 659 |
| BOC | 91653 |
| BSG | 682 |
| BST1 | 683 |
| BST2 | 684 |
| BTC | 685 |
| BTLA | 151888 |
| BTN1A1 | 696 |
| BTN2A1 | 11120 |
| BTN2A2 | 10385 |
| BTN2A3P | 54718 |
| BTN3A1 | 11119 |
| BTN3A2 | 11118 |
| BTN3A3 | 10384 |
| BTNL2 | 56244 |
| BTNL3 | 10917 |
| BTNL8 | 79908 |
| BTNL9 | 153579 |
| BUTR1 | 100129094 |
| C10orf26 | 54838 |
| C10orf35 | 219738 |
| C10orf54 | 64115 |
| C11orf24 | 53838 |
| C11orf87 | 399947 |
| C11orf92 | 399948 |
| C12orf53 | 196500 |
| C12orf59 | 120939 |
| C14orf132 | 100132684 |
| C14orf180 | 400258 |
| C14orf37 | 145407 |
| C15orf24 | 56851 |
| C16orf54 | 728070 |
| C16orf91 | 283951 |
| C16orf92 | 146378 |
| C17orf80 | 55028 |
| C18orf1 | 753 |
| C19orf18 | 147685 |
| C19orf24 | 55009 |
| C19orf38 | 255809 |
| C19orf63 | 284361 |
| C19orf75 | 284369 |
| C1orf101 | 257044 |
| C1orf130 | 400746 |
| C1orf159 | 54991 |
| C1orf85 | 112770 |
| C2orf82 | 389084 |
| C2orf89 | 129293 |
| C3orf18 | 51161 |
| C3orf35 | 339883 |
| C3orf45 | 132228 |
| C4orf32 | 132720 |
| C4orf34 | 201895 |
| C5orf15 | 56951 |
| C6orf25 | 80739 |
| C6orf72 | 116254 |
| C9orf11 | 54586 |
| CA12 | 771 |
| CA14 | 23632 |
| CA4 | 762 |
| CA9 | 768 |
| CACHD1 | 57685 |
| CACNA2D3 | 55799 |
| CACNA2D4 | 93589 |
| CADM1 | 23705 |
| CADM2 | 253559 |
| CADM3 | 57863 |
| CADM4 | 199731 |
| CATSPERD | 257062 |
| CATSPERG | 57828 |
| CAV3 | 859 |
| CCDC107 | 203260 |
| CCDC47 | 57003 |
| CD101 | 9398 |
| CD109 | 135228 |
| CD14 | 929 |
| CD160 | 11126 |
| CD163 | 9332 |
| CD164 | 8763 |
| CD164L2 | 388611 |
| CD177 | 57126 |
| CD180 | 4064 |
| CD19 | 930 |
| CD1A | 909 |
| CD1B | 910 |
| CD1C | 911 |
| CD1D | 912 |
| CD1E | 913 |
| CD2 | 914 |
| CD200 | 4345 |
| CD200R1 | 131450 |
| CD200R1L | 344807 |
| CD22 | 933 |
| CD226 | 10666 |
| CD24 | 100133941 |
| CD244 | 51744 |
| CD247 | 919 |
| CD248 | 57124 |
| CD27 | 939 |
| CD274 | 29126 |
| CD276 | 80381 |
| CD28 | 940 |
| CD300A | 11314 |
| CD300C | 10871 |
| CD300E | 342510 |
| CD300LB | 124599 |
| CD300LD | 100131439 |
| CD300LF | 146722 |
| CD300LG | 146894 |
| CD302 | 9936 |
| CD320 | 51293 |
| CD33 | 945 |
| CD34 | 947 |
| CD3D | 915 |
| CD3E | 916 |
| CD3G | 917 |
| CD4 | 920 |
| CD40 | 958 |
| CD44 | 960 |
| CD46 | 4179 |
| CD47 | 961 |
| CD48 | 962 |
| CD5 | 921 |
| CD52 | 1043 |
| CD55 | 1604 |
| CD58 | 965 |
| CD6 | 923 |
| CD68 | 968 |
| CD7 | 924 |
| CD79A | 973 |
| CD79B | 974 |
| CD80 | 941 |
| CD83 | 9308 |
| CD84 | 8832 |
| CD86 | 942 |
| CD8A | 925 |
| CD8B | 926 |
| CD93 | 22918 |
| CD96 | 10225 |
| CD99 | 4267 |
| CD99L2 | 83692 |
| CDCP1 | 64866 |
| CDH1 | 999 |
| CDH10 | 1008 |
| CDH11 | 1009 |
| CDH12 | 1010 |
| CDH13 | 1012 |
| CDH15 | 1013 |
| CDH16 | 1014 |
| CDH17 | 1015 |
| CDH18 | 1016 |
| CDH19 | 28513 |
| CDH2 | 1000 |
| CDH20 | 28316 |
| CDH22 | 64405 |
| CDH24 | 64403 |
| CDH26 | 60437 |
| CDH3 | 1001 |
| CDH4 | 1002 |
| CDH5 | 1003 |
| CDH6 | 1004 |
| CDH7 | 1005 |
| CDH8 | 1006 |
| CDH9 | 1007 |
| CDHR3 | 222256 |
| CDHR5 | 53841 |
| CDON | 50937 |
| CEACAM1 | 634 |
| CEACAM16 | 388551 |
| CEACAM18 | 729767 |
| CEACAM19 | 56971 |
| CEACAM20 | 125931 |
| CEACAM21 | 90273 |
| CEACAM3 | 1084 |
| CEACAM4 | 1089 |
| CEACAM5 | 1048 |
| CEACAM6 | 4680 |
| CEACAM7 | 1087 |
| CEACAM8 | 1088 |
| CHL1 | 10752 |
| CHODL | 140578 |
| CILP | 8483 |
| CILP2 | 148113 |
| CLCA2 | 9635 |
| CLCA4 | 22802 |
| CLEC14A | 161198 |
| CLMP | 79827 |
| CLSTN1 | 22883 |
| CLSTN2 | 64084 |
| CLSTN3 | 9746 |
| CNTFR | 1271 |
| CNTN1 | 1272 |
| CNTN2 | 6900 |
| CNTN3 | 5067 |
| CNTN4 | 152330 |
| CNTN5 | 53942 |
| CNTN6 | 27255 |
| CNTNAP1 | 8506 |
| CNTNAP2 | 26047 |
| CNTNAP3 | 79937 |
| CNTNAP4 | 85445 |
| CNTNAP5 | 129684 |
| CPM | 1368 |
| CR1 | 1378 |
| CR2 | 1380 |
| CRB1 | 23418 |
| CRB2 | 286204 |
| CRB3 | 92359 |
| CRIM1 | 51232 |
| CRLF1 | 9244 |
| CRLF2 | 64109 |
| CRTAM | 56253 |
| CSF1 | 1435 |
| CSF1R | 1436 |
| CSF2RA | 1438 |
| CSF2RB | 1439 |
| CSF3R | 1441 |
| CSPG4 | 1464 |
| CSPG5 | 10675 |
| CTLA4 | 1493 |
| CUZD1 | 50624 |
| CX3CL1 | 6376 |
| CXADR | 1525 |
| CXCL16 | 58191 |
| CXorf68 | 100132963 |
| CYYR1 | 116159 |
| DAG1 | 1605 |
| DCBLD1 | 285761 |
| DCBLD2 | 131566 |
| DCC | 1630 |
| DDOST | 1650 |
| DDR1 | 780 |
| DDR2 | 4921 |
| DGCR2 | 9993 |
| DLK1 | 8788 |
| DLK2 | 65989 |
| DLL1 | 28514 |
| DLL3 | 10683 |
| DLL4 | 54567 |
| DNER | 92737 |
| DPCR1 | 135656 |
| DPEP1 | 1800 |
| DPEP2 | 64174 |
| DPEP3 | 64180 |
| DSC1 | 1823 |
| DSC2 | 1824 |
| DSC3 | 1825 |
| DSCAM | 1826 |
| DSCAML1 | 57453 |
| DSG1 | 1828 |
| DSG2 | 1829 |
| DSG3 | 1830 |
| DSG4 | 147409 |
| DTPQ5903 | 147645 |
| ECSM2 | 641700 |
| EDA2R | 60401 |
| EDAR | 10913 |
| EFNA3 | 1944 |
| EFNA5 | 1946 |
| EFNB1 | 1947 |
| EFNB2 | 1948 |
| EFNB3 | 1949 |
| EGF | 1950 |
| EGFR | 1956 |
| ELFN1 | 392617 |
| ELFN2 | 114794 |
| EMB | 133418 |
| EMCN | 51705 |
| ENG | 2022 |
| ENPP5 | 59084 |
| EPCAM | 4072 |
| EPGN | 255324 |
| EPHA1 | 2041 |
| EPHA10 | 284656 |
| EPHA2 | 1969 |
| EPHA3 | 2042 |
| EPHA4 | 2043 |
| EPHA5 | 2044 |
| EPHA6 | 285220 |
| EPHA7 | 2045 |
| EPHA8 | 2046 |
| EPHB1 | 2047 |
| EPHB2 | 2048 |
| EPHB3 | 2049 |
| EPHB4 | 2050 |
| EPHB6 | 2051 |
| EPOR | 2057 |
| ERBB2 | 2064 |
| ERBB3 | 2065 |
| ERBB4 | 2066 |
| EREG | 2069 |
| ERMAP | 114625 |
| ERN2 | 10595 |
| ESAM | 90952 |
| EVA1C | 59271 |
| EVC2 | 132884 |
| EVI2A | 2123 |
| EVI2B | 2124 |
| F11R | 50848 |
| F3 | 2152 |
| FAIM3 | 9214 |
| FAM171A1 | 221061 |
| FAM171B | 165215 |
| FAM174A | 345757 |
| FAM174B | 400451 |
| FAM187A | 66784 |
| FAM187B | 148109 |
| FAM189A2 | 9413 |
| FAM200A | 221786 |
| FAM209A | 200232 |
| FAM209B | 388799 |
| FAS | 355 |
| FCAMR | 83953 |
| FCAR | 2204 |
| FCER1A | 2205 |
| FCGR1A | 2209 |
| FCGR1B | 2210 |
| FCGR1C | 100132417 |
| FCGR2A | 2212 |
| FCGR2B | 2213 |
| FCGR2C | 9103 |
| FCGR3A | 2214 |
| FCGR3B | 2215 |
| FCGRT | 2217 |
| FCRL1 | 115350 |
| FCRL2 | 79368 |
| FCRL3 | 115352 |
| FCRL4 | 83417 |
| FCRL5 | 83416 |
| FCRL6 | 343413 |
| FCRLA | 84824 |
| FCRLB | 127943 |
| FGFR1 | 2260 |
| FGFR2 | 2263 |
| FGFR3 | 2261 |
| FGFR4 | 2264 |
| FGFRL1 | 53834 |
| FLRT1 | 23769 |
| FLRT2 | 23768 |
| FLRT3 | 23767 |
| FLT1 | 2321 |
| FLT3 | 2322 |
| FLT3LG | 2323 |
| FLT4 | 2324 |
| FNDC3A | 22862 |
| FNDC4 | 64838 |
| FNDC9 | 408263 |
| FOLR1 | 2348 |
| FOLR2 | 2350 |
| FRRS1L | 23732 |
| FSTL4 | 23105 |
| FSTL5 | 56884 |
| FURIN | 5045 |
| FXYD5 | 53827 |
| GAS1 | 2619 |
| GFRA1 | 2674 |
| GFRA2 | 2675 |
| GFRA3 | 2676 |
| GFRA4 | 64096 |
| GFRAL | 389400 |
| GHR | 2690 |
| GLG1 | 2734 |
| GLIPR1 | 11010 |
| GLIPR1L2 | 144321 |
| GML | 2765 |
| GP1BA | 2811 |
| GP1BB | 2812 |
| GP2 | 2813 |
| GP5 | 2814 |
| GP6 | 51206 |
| GP9 | 2815 |
| GPA33 | 10223 |
| GPC1 | 2817 |
| GPC2 | 221914 |
| GPC3 | 2719 |
| GPC4 | 2239 |
| GPC6 | 10082 |
| GPIHBP1 | 338328 |
| GPNMB | 10457 |
| GPR116 | 221395 |
| GPR124 | 25960 |
| GPR125 | 166647 |
| GUCY2C | 2984 |
| GUCY2D | 3000 |
| GUCY2F | 2986 |
| GYPA | 2993 |
| GYPB | 2994 |
| GYPC | 2995 |
| GYPE | 2996 |
| HAPLN2 | 60484 |
| HAPLN3 | 145864 |
| HAPLN4 | 404037 |
| HAVCR1 | 26762 |
| HAVCR2 | 84868 |
| HBEGF | 1839 |
| HCST | 10870 |
| HEG1 | 57493 |
| HEPACAM | 220296 |
| HEPACAM2 | 253012 |
| HEPH | 9843 |
| HEPHL1 | 341208 |
| HFE | 3077 |
| HFE2 | 148738 |
| HHLA2 | 11148 |
| HYAL2 | 8692 |
| ICAM1 | 3383 |
| ICAM2 | 3384 |
| ICAM3 | 3385 |
| ICAM4 | 3386 |
| ICAM5 | 7087 |
| ICOS | 29851 |
| ICOSLG | 23308 |
| IFNAR1 | 3454 |
| IFNAR2 | 3455 |
| IFNGR1 | 3459 |
| IFNGR2 | 3460 |
| IFNLR1 | 163702 |
| IGDCC3 | 9543 |
| IGDCC4 | 57722 |
| IGF1R | 3480 |
| IGF2R | 3482 |
| IGFBP7 | 3490 |
| IGFBPL1 | 347252 |
| IGFLR1 | 79713 |
| IGLON5 | 402665 |
| IGSF1 | 3547 |
| IGSF11 | 152404 |
| IGSF21 | 84966 |
| IGSF3 | 3321 |
| IGSF5 | 150084 |
| IGSF6 | 10261 |
| IGSF8 | 93185 |
| IGSF9 | 57549 |
| IGSF9B | 22997 |
| IL10RA | 3587 |
| IL10RB | 3588 |
| IL11RA | 3590 |
| IL12B | 3593 |
| IL12RB1 | 3594 |
| IL12RB2 | 3595 |
| IL13RA1 | 3597 |
| IL13RA2 | 3598 |
| IL15RA | 3601 |
| IL17RA | 23765 |
| IL17RB | 55540 |
| IL17RC | 84818 |
| IL17RD | 54756 |
| IL17RE | 132014 |
| IL18BP | 10068 |
| IL18R1 | 8809 |
| IL18RAP | 8807 |
| IL1R1 | 3554 |
| IL1R2 | 7850 |
| IL1RAP | 3556 |
| IL1RAPL1 | 11141 |
| IL1RAPL2 | 26280 |
| IL1RL1 | 9173 |
| IL1RL2 | 8808 |
| IL20RA | 53832 |
| IL20RB | 53833 |
| IL21R | 50615 |
| IL22RA1 | 58985 |
| IL23R | 149233 |
| IL27RA | 9466 |
| IL2RA | 3559 |
| IL2RB | 3560 |
| IL2RG | 3561 |
| IL31RA | 133396 |
| IL3RA | 3563 |
| IL4R | 3566 |
| IL5RA | 3568 |
| IL6R | 3570 |
| IL6ST | 3572 |
| IL7R | 3575 |
| IL9R | 3581 |
| ILDR1 | 286676 |
| ILDR2 | 387597 |
| IMPG2 | 50939 |
| INSR | 3643 |
| INSRR | 3645 |
| ISLR | 3671 |
| ISLR2 | 57611 |
| ITFG1 | 81533 |
| ITGA1 | 3672 |
| ITGA10 | 8515 |
| ITGA11 | 22801 |
| ITGA2 | 3673 |
| ITGA2B | 3674 |
| ITGA3 | 3675 |
| ITGA4 | 3676 |
| ITGA5 | 3678 |
| ITGA6 | 3655 |
| ITGA7 | 3679 |
| ITGA8 | 8516 |
| ITGA9 | 3680 |
| ITGAD | 3681 |
| ITGAE | 3682 |
| ITGAL | 3683 |
| ITGAM | 3684 |
| ITGAV | 3685 |
| ITGAX | 3687 |
| ITLN1 | 55600 |
| IZUMO1 | 284359 |
| IZUMO2 | 126123 |
| JAG1 | 182 |
| JAG2 | 3714 |
| JAM2 | 58494 |
| JAM3 | 83700 |
| JTB | 10899 |
| KAZALD1 | 81621 |
| KCNE4 | 23704 |
| KDR | 3791 |
| KIAA0090 | 23065 |
| KIAA0319 | 9856 |
| KIAA0319L | 79932 |
| KIAA1024 | 23251 |
| KIAA1324 | 57535 |
| KIAA1324L | 222223 |
| KIAA1644 | 85352 |
| KIR2DL1 | 3802 |
| KIR2DL2 | 3803 |
| KIR2DL3 | 3804 |
| KIR2DL4 | 3805 |
| KIR2DL5A | 57292 |
| KIR2DL5B | 553128 |
| KIR2DS1 | 3806 |
| KIR2DS2 | 100132285 |
| KIR2DS3 | 3808 |
| KIR2DS4 | 3809 |
| KIR2DS5 | 3810 |
| KIR3DL1 | 3811 |
| KIR3DL2 | 3812 |
| KIR3DL3 | 115653 |
| KIR3DP1 | 548594 |
| KIR3DS1 | 3813 |
| KIR3DX1 | 90011 |
| KIRREL | 55243 |
| KIRREL2 | 84063 |
| KIRREL3 | 84623 |
| KIT | 3815 |
| KITLG | 4254 |
| KL | 9365 |
| KLB | 152831 |
| KLRAP1 | 10748 |
| KREMEN1 | 83999 |
| KREMEN2 | 79412 |
| L1CAM | 3897 |
| LAG3 | 3902 |
| LAIR1 | 3903 |
| LAIR2 | 3904 |
| LAMP1 | 3916 |
| LAMP2 | 3920 |
| LAMP3 | 27074 |
| LAMP5 | 24141 |
| LAX1 | 54900 |
| LAYN | 143903 |
| LCTL | 197021 |
| LDLR | 3949 |
| LDLRAD2 | 401944 |
| LDLRAD3 | 143458 |
| LEPR | 3953 |
| LIFR | 3977 |
| LILRA1 | 11024 |
| LILRA2 | 11027 |
| LILRA3 | 11026 |
| LILRA4 | 23547 |
| LILRA5 | 353514 |
| LILRA6 | 79168 |
| LILRB1 | 10859 |
| LILRB2 | 10288 |
| LILRB3 | 11025 |
| LILRB4 | 11006 |
| LILRB5 | 10990 |
| LINGO1 | 84894 |
| LINGO2 | 158038 |
| LINGO3 | 645191 |
| LINGO4 | 339398 |
| LMLN | 89782 |
| LOC160348 | 160348 |
| LOC256223 | 256223 |
| LOC374383 | 374383 |
| LOC376666 | 376666 |
| LOC652900 | 652900 |
| LRCH4 | 4034 |
| LRFN1 | 57622 |
| LRFN2 | 57497 |
| LRFN3 | 79414 |
| LRFN4 | 78999 |
| LRFN5 | 145581 |
| LRIG1 | 26018 |
| LRIG2 | 9860 |
| LRIG3 | 121227 |
| LRIT1 | 26103 |
| LRIT2 | 340745 |
| LRIT3 | 345193 |
| LRP10 | 26020 |
| LRP11 | 84918 |
| LRP12 | 29967 |
| LRP3 | 4037 |
| LRP4 | 4038 |
| LRP5 | 4041 |
| LRP6 | 4040 |
| LRP8 | 7804 |
| LRPAP1 | 4043 |
| LRRC15 | 131578 |
| LRRC19 | 64922 |
| LRRC24 | 441381 |
| LRRC25 | 126364 |
| LRRC26 | 389816 |
| LRRC3 | 81543 |
| LRRC32 | 2615 |
| LRRC33 | 375387 |
| LRRC37A | 9884 |
| LRRC37A2 | 474170 |
| LRRC37A3 | 374819 |
| LRRC37B | 114659 |
| LRRC38 | 126755 |
| LRRC3B | 116135 |
| LRRC4 | 64101 |
| LRRC4B | 94030 |
| LRRC4C | 57689 |
| LRRC52 | 440699 |
| LRRC55 | 219527 |
| LRRC66 | 339977 |
| LRRN1 | 57633 |
| LRRN2 | 10446 |
| LRRN3 | 54674 |
| LRRN4 | 164312 |
| LRRN4CL | 221091 |
| LRRTM1 | 347730 |
| LRRTM2 | 26045 |
| LRRTM3 | 347731 |
| LRRTM4 | 80059 |
| LRTM1 | 57408 |
| LRTM2 | 654429 |
| LSAMP | 4045 |
| LSR | 51599 |
| LTBR | 4055 |
| LTK | 4058 |
| LY6D | 8581 |
| LY6E | 4061 |
| LY6G6C | 80740 |
| LY6G6D | 58530 |
| LY6G6F | 259215 |
| LY6H | 4062 |
| LY6K | 54742 |
| LY75 | 4065 |
| LY9 | 4063 |
| LYPD1 | 116372 |
| LYPD3 | 27076 |
| LYPD5 | 284348 |
| LYPD6 | 130574 |
| LYPD6B | 130576 |
| LYSMD3 | 116068 |
| LYSMD4 | 145748 |
| LYVE1 | 10894 |
| M6PR | 4074 |
| MADCAM1 | 8174 |
| MAG | 4099 |
| MAMDC4 | 158056 |
| MANSC1 | 54682 |
| MCAM | 4162 |
| MDGA1 | 266727 |
| MDGA2 | 161357 |
| MEGF10 | 84466 |
| MEGF11 | 84465 |
| MEGF8 | 1954 |
| MEGF9 | 1955 |
| MEP1A | 4224 |
| MEP1B | 4225 |
| MERTK | 10461 |
| MET | 4233 |
| MFAP3 | 4238 |
| MFAP3L | 9848 |
| MFI2 | 4241 |
| MICA | 4276 |
| MICB | 4277 |
| MILR1 | 28402 |
| MMGT1 | 93380 |
| MMP14 | 4323 |
| MMP15 | 4324 |
| MMP16 | 4325 |
| MMP24 | 10893 |
| MOG | 4340 |
| MPEG1 | 219972 |
| MPL | 4352 |
| MPZ | 4359 |
| MPZL1 | 9019 |
| MPZL2 | 10205 |
| MPZL3 | 196264 |
| MR1 | 3140 |
| MRC1 | 4360 |
| MRC2 | 9902 |
| MSLN | 10232 |
| MST1R | 4486 |
| MUC1 | 4582 |
| MUC13 | 56667 |
| MUC15 | 143662 |
| MUC21 | 394263 |
| MUSK | 4593 |
| MXRA5 | 25878 |
| MXRA7 | 439921 |
| MXRA8 | 54587 |
| MYEOV | 26579 |
| NAGPA | 51172 |
| NCAM1 | 4684 |
| NCAM2 | 4685 |
| NCAN | 1463 |
| NCLN | 56926 |
| NCR1 | 9437 |
| NCR2 | 9436 |
| NCR3 | 259197 |
| NCSTN | 23385 |
| NEGR1 | 257194 |
| NEO1 | 4756 |
| NETO1 | 81832 |
| NETO2 | 81831 |
| NFAM1 | 150372 |
| NFASC | 23114 |
| NGFR | 4804 |
| NLGN1 | 22871 |
| NLGN2 | 57555 |
| NLGN3 | 54413 |
| NLGN4X | 57502 |
| NLGN4Y | 22829 |
| NOMO1 | 23420 |
| NOMO3 | 408050 |
| NOTCH1 | 4851 |
| NOTCH2 | 4853 |
| NOTCH3 | 4854 |
| NOTCH4 | 4855 |
| NPDC1 | 56654 |
| NPHS1 | 4868 |
| NPR1 | 4881 |
| NPR2 | 4882 |
| NPR3 | 4883 |
| NPTN | 27020 |
| NRCAM | 4897 |
| NRG1 | 3084 |
| NRG2 | 9542 |
| NRG4 | 145957 |
| NRN1 | 51299 |
| NRN1L | 123904 |
| NRP1 | 8829 |
| NRP2 | 8828 |
| NRXN1 | 9378 |
| NRXN2 | 9379 |
| NRXN3 | 9369 |
| NT5E | 4907 |
| NTM | 50863 |
| NTNG1 | 22854 |
| NTRK1 | 4914 |
| NTRK2 | 4915 |
| NTRK3 | 4916 |
| OMG | 4974 |
| OPCML | 4978 |
| OSCAR | 126014 |
| OSMR | 9180 |
| OSTM1 | 28962 |
| OTOA | 146183 |
| P2RX2 | 22953 |
| PAPLN | 89932 |
| PARM1 | 25849 |
| PCDHGB6 | 56100 |
| PCDHGC3 | 5098 |
| PCDHGC5 | 93708 |
| PCSK4 | 54760 |
| PCSK7 | 9159 |
| PDCD1 | 5133 |
| PDCD1LG2 | 80380 |
| PDGFRA | 5156 |
| PDGFRB | 5159 |
| PDGFRL | 5157 |
| PDPN | 10630 |
| PEAR1 | 375033 |
| PECAM1 | 5175 |
| Phospholipase | |
| Inhibitor | |
| PI16 | 221476 |
| PIGR | 5284 |
| PIK3IP1 | 113791 |
| PILRA | 29992 |
| PILRB | 29990 |
| PLA2R1 | 22925 |
| PLB1 | 151056 |
| PLXDC1 | 57125 |
| PLXDC2 | 84898 |
| PLXNA1 | 5361 |
| PLXNA2 | 5362 |
| PLXNA3 | 55558 |
| PLXNA4 | 91584 |
| PLXNB1 | 5364 |
| PLXNB2 | 23654 |
| PLXNB3 | 5365 |
| PLXNC1 | 10154 |
| PLXND1 | 23129 |
| PMEL | 6490 |
| PMEPA1 | 56937 |
| PODXL | 5420 |
| PODXL2 | 50512 |
| PRIMA1 | 145270 |
| PRLR | 5618 |
| PRND | 23627 |
| PRNP | 5621 |
| PROCR | 10544 |
| PRRG2 | 5639 |
| PRRG3 | 79057 |
| PRRG4 | 79056 |
| PRSS21 | 10942 |
| PRSS41 | 360226 |
| PRSS55 | 203074 |
| PRSS8 | 5652 |
| PRTG | 283659 |
| PSG1 | 5669 |
| PSG2 | 5670 |
| PSG3 | 5671 |
| PSG4 | 5672 |
| PSG5 | 5673 |
| PSG6 | 5675 |
| PSG7 | 5676 |
| PSG8 | 440533 |
| PSG9 | 5678 |
| PTCRA | 171558 |
| PTGFRN | 5738 |
| PTK7 | 5754 |
| PTPRA | 5786 |
| PTPRB | 5787 |
| PTPRC | 5788 |
| PTPRCAP | 5790 |
| PTPRD | 5789 |
| PTPRE | 5791 |
| PTPRF | 5792 |
| PTPRG | 5793 |
| PTPRH | 5794 |
| PTPRJ | 5795 |
| PTPRK | 5796 |
| PTPRM | 5797 |
| PTPRN | 5798 |
| PTPRN2 | 5799 |
| PTPRO | 5800 |
| PTPRR | 5801 |
| PTPRS | 5802 |
| PTPRT | 11122 |
| PTPRU | 10076 |
| PTPRZ1 | 5803 |
| PVR | 5817 |
| PVRL1 | 5818 |
| PVRL2 | 5819 |
| NECTIN3 | 25945 |
| PVRL4 | 81607 |
| PXDN | 7837 |
| PXDNL | 137902 |
| QSOX1 | 5768 |
| QSOX2 | 169714 |
| RAET1E | 135250 |
| RAET1G | 353091 |
| RAET1L | 154064 |
| RAMP1 | 10267 |
| RAMP2 | 10266 |
| RAMP3 | 10268 |
| RECK | 8434 |
| RELL1 | 768211 |
| RELT | 84957 |
| RET | 5979 |
| RGMA | 56963 |
| RGMB | 285704 |
| ROBO1 | 6091 |
| ROBO2 | 6092 |
| ROBO3 | 64221 |
| ROBO4 | 54538 |
| ROR1 | 4919 |
| ROR2 | 4920 |
| ROS1 | 6098 |
| RPN1 | 6184 |
| RPRML | 388394 |
| RTN4R | 65078 |
| RTN4RL1 | 146760 |
| RYK | 6259 |
| SARAF | 51669 |
| SCARF1 | 8578 |
| SCARF2 | 91179 |
| SCN1B | 6324 |
| SCN2B | 6327 |
| SCN3B | 55800 |
| SCN4B | 6330 |
| SDC1 | 6382 |
| SDC2 | 6383 |
| SDC3 | 9672 |
| SDC4 | 6385 |
| SDK1 | 221935 |
| SDK2 | 237979 |
| SECTM1 | 6398 |
| SEL1L3 | 23231 |
| SELE | 6401 |
| SELL | 6402 |
| SELP | 6403 |
| SELPLG | 6404 |
| SEMA3A | 10371 |
| SEMA3B | 7869 |
| SEMA3C | 10512 |
| SEMA3D | 223117 |
| SEMA3E | 9723 |
| SEMA3F | 6405 |
| SEMA3G | 56920 |
| SEMA4A | 64218 |
| SEMA4B | 10509 |
| SEMA4C | 54910 |
| SEMA4D | 10507 |
| SEMA4F | 10505 |
| SEMA4G | 57715 |
| SEMA5A | 9037 |
| SEMA5B | 54437 |
| SEMA6A | 57556 |
| SEMA6B | 10501 |
| SEMA6C | 10500 |
| SEMA6D | 80031 |
| SEMA7A | 8482 |
| SERTM1 | 400120 |
| SEZ6 | 124925 |
| SEZ6L | 23544 |
| SEZ6L2 | 26470 |
| SGCA | 6442 |
| SGCE | 8910 |
| SHISA2 | 387914 |
| SHISA3 | 152573 |
| SHISA4 | 149345 |
| SHISA5 | 51246 |
| SIGIRR | 59307 |
| SIGLEC1 | 6614 |
| SIGLEC10 | 89790 |
| SIGLEC11 | 114132 |
| SIGLEC12 | 89858 |
| SIGLEC14 | 100049587 |
| SIGLEC15 | 284266 |
| SIGLEC16 | 400709 |
| SIGLEC5 | 8778 |
| SIGLEC6 | 946 |
| SIGLEC7 | 27036 |
| SIGLEC8 | 27181 |
| SIGLEC9 | 27180 |
| SIRPA | 140885 |
| SIRPB1 | 10326 |
| SIRPB2 | 284759 |
| SIRPD | 128646 |
| SIRPG | 55423 |
| SIT1 | 27240 |
| SKINTL | 391037 |
| SLAMF1 | 6504 |
| SLAMF6 | 114836 |
| SLAMF7 | 57823 |
| SLAMF8 | 56833 |
| SLAMF9 | 89886 |
| SLITRK1 | 114798 |
| SLITRK2 | 84631 |
| SLITRK3 | 22865 |
| SLITRK4 | 139065 |
| SLITRK5 | 26050 |
| SLITRK6 | 84189 |
| SMAGP | 57228 |
| SOGA3 | 387104 |
| SORCS1 | 114815 |
| SORCS2 | 57537 |
| SORCS3 | 22986 |
| SORL1 | 6653 |
| SORT1 | 6272 |
| SPACA1 | 81833 |
| SPACA4 | 171169 |
| SPATA9 | 83890 |
| SPINT1 | 6692 |
| SPINT2 | 10653 |
| SPN | 6693 |
| SPRN | 503542 |
| STAB1 | 23166 |
| STAB2 | 55576 |
| STIM1 | 6786 |
| SUSD1 | 64420 |
| SUSD2 | 56241 |
| SUSD3 | 203328 |
| SUSD4 | 55061 |
| SUSD5 | 26032 |
| TACSTD2 | 4070 |
| TAPBP | 6892 |
| TAPBPL | 55080 |
| TARM1 | 441864 |
| TCP11 | 6954 |
| TCTN2 | 79867 |
| TCTN3 | 26123 |
| TDGF1 | 6997 |
| TECTA | 7007 |
| TECTB | 6975 |
| TEK | 7010 |
| TEX101 | 83639 |
| TEX29 | 121793 |
| TGFA | 7039 |
| TGFBR1 | 7046 |
| TGFBR2 | 7048 |
| TGFBR3 | 7049 |
| TGOLN2 | 10618 |
| THBD | 7056 |
| THSD1 | 55901 |
| THSD7A | 221981 |
| THSD7B | 80731 |
| THY1 | 7070 |
| TIE1 | 7075 |
| TIGIT | 201633 |
| TIMD4 | 91937 |
| TLR1 | 7096 |
| TLR10 | 81793 |
| TLR2 | 7097 |
| TLR3 | 7098 |
| TLR4 | 7099 |
| TLR5 | 7100 |
| TLR6 | 10333 |
| TLR7 | 51284 |
| TLR8 | 51311 |
| TLR9 | 54106 |
| TMEFF1 | 8577 |
| TMEFF2 | 23671 |
| TMEM108 | 66000 |
| TMEM119 | 338773 |
| TMEM123 | 114908 |
| TMEM130 | 222865 |
| TMEM132A | 54972 |
| TMEM132B | 114795 |
| TMEM132D | 121256 |
| TMEM132E | 124842 |
| TMEM154 | 201799 |
| TMEM156 | 80008 |
| TMEM158 | 25907 |
| TMEM167A | 153339 |
| TMEM167B | 56900 |
| TMEM183A | 92703 |
| TMEM183B | 653659 |
| TMEM190 | 147744 |
| TMEM207 | 131920 |
| TMEM213 | 155006 |
| TMEM240 | 339453 |
| TMEM25 | 84866 |
| TMEM27 | 57393 |
| TMEM52 | 339456 |
| TMEM59 | 9528 |
| TMEM59L | 25789 |
| TMEM81 | 388730 |
| TMEM92 | 162461 |
| TMEM95 | 339168 |
| TMEM9B | 56674 |
| TMIE | 259236 |
| TMIGD1 | 388364 |
| TMIGD2 | 126259 |
| TMPRSS12 | 283471 |
| TMX4 | 56255 |
| TNFRSF10A | 8797 |
| TNFRSF10B | 8795 |
| TNFRSF10C | 8794 |
| TNFRSF10D | 8793 |
| TNFRSF11A | 8792 |
| TNFRSF11B | 4982 |
| TNFRSF12A | 51330 |
| TNFRSF13B | 23495 |
| TNFRSF13C | 115650 |
| TNFRSF14 | 8764 |
| TNFRSF17 | 608 |
| TNFRSF18 | 8784 |
| TNFRSF19 | 55504 |
| TNFRSF1A | 7132 |
| TNFRSF1B | 7133 |
| TNFRSF21 | 27242 |
| TNFRSF25 | 8718 |
| TNFRSF4 | 7293 |
| TNFRSF6B | 8771 |
| TNFRSF8 | 943 |
| TNFRSF9 | 3604 |
| TNFSF15 | 9966 |
| TP53\|13 | 90313 |
| TPBG | 7162 |
| TPO | 7173 |
| TPSG1 | 25823 |
| TREH | 11181 |
| TREM1 | 54210 |
| TREM2 | 54209 |
| TREML1 | 340205 |
| TREML2 | 79865 |
| TREML4 | 285852 |
| TRIL | 9865 |
| TXNDC15 | 79770 |
| TYRO3 | 7301 |
| TYROBP | 7305 |
| ULBP1 | 80329 |
| ULBP2 | 80328 |
| ULBP3 | 79465 |
| UMOD | 7369 |
| UMODL1 | 89766 |
| UNC5A | 90249 |
| UNC5B | 219699 |
| UNC5C | 8633 |
| UNC5D | 137970 |
| UPK3BL | 100134938 |
| VASN | 114990 |
| VCAM1 | 7412 |
| VLDLR | 7436 |
| VNN1 | 8876 |
| VNN2 | 8875 |
| VNN3 | 55350 |
| VPREB1 | 7441 |
| VPREB3 | 29802 |
| VSIG1 | 340547 |
| VSIG10 | 54621 |
| VSIG2 | 23584 |
| VSIG4 | 11326 |
| VSTM1 | 284415 |
| VSTM2A | 222008 |
| VSTM2B | 342865 |
| VSTM2L | 128434 |
| VSTM4 | 196740 |
| VSTM5 | 387804 |
| VTCN1 | 79679 |
| WFIKKN1 | 117166 |
| WFIKKN2 | 124857 |
| XG | 7499 |
| XPNPEP2 | 7512 |
| ZAN | 7455 |
| ZP1 | 22917 |
| ZP2 | 7783 |
| ZP3 | 7784 |
| ZP4 | 57829 |
| ZPBP | 11055 |
| ZPBP2 | 124626 |
| ZPLD1 | 131368 |

In some aspects, the collection of target polypeptides comprises the extracellular domains of at least 5%, at least 6%, at least 7%, at least 8%, at least 9%, at least 10%, at least 11%, at least 12%, at least 13%, at least 14%, at least 15%, at least 16%, at least 17%, at least 18%, at least 19%, at least 20%, at least 21%, at least 22%, at least 23%, at least 24%, at least 25%, at least 26%, at least 27%, at least 28%, at least 29%, at least 30%, at least 31%, at least 32%, at least 33%, at least 34%, at least 35%, at least 36%, at least 37%, at least 38%, at least 39%, at least 40%, at least 41%, at least 42%, at least 43%, at least 44%, at least 45%, at least 46%, at least 47%, at least 48%, at least 49%, at least 50%, at least 51%, at least 52%, at least 53%, at least 54%, at least 55%, at least 56%, at least 57%, at least 58%, at least 59%, at least 60%, at least 61%, at least 62%, at least 63%, at least 64%, at least 65%, at least 66%, at least 67%, at least 68%, at least 69%, at least 70%, at least 71%, at least 72%, at least 73%, at least 74%, at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% of the proteins of Table 4, e.g., 5%-15%, 15%-25%, 25%-35%, 35%-455, 45%-55%, 55%-65%, 65%-75%, 75%-85%, 85%-95%, or 95%-100% of the proteins of Table 4.

In some aspects, the collection of target polypeptides comprises the extracellular domains of at least 100, at least 150, at least 200, at least 250, at least 300, at least 350, at least 400, at least 450, at least 500, at least 550, at least 600, at least 650, at least 700, at least 750, at least 800, at least 850, at least 900, at least 950, at least 1000, at least 1050, at least 1100, at least 1150, or all 1195 of the proteins of Table 4, e.g., comprises the extracellular domains of 100-150, 150-200, 200-250, 250-300, 300-350, 350-400, 400-450, 450-500, 500-550, 550-600, 600-650, 650-700, 750-800, 800-850, 850-900, 900-950, 950-1000, 1000-1050, 1050-1100, 1100-1150, or all 1195 of the polypeptides of Table 4.

In some aspects, the collection of target polypeptides comprises the extracellular domains of at least 25% of the proteins of Table 4. In some aspects, the collection of target polypeptides comprises the extracellular domains of at least 50% of the proteins of Table 4. In some aspects, the collection of target polypeptides comprises the extracellular domains of at least 75% of the proteins of Table 4. In some aspects, the collection of target polypeptides comprises the extracellular domains of at least 90% of the proteins of Table 4. In some aspects, the collection of target polypeptides comprises the extracellular domains of all of the proteins of Table 4.

In other aspects, the receptors are multi-transmembrane receptors (MTMRs), e.g., members of the GPCR superfamily.

E. Assays for Interaction

To perform the protein-protein interaction assay, the collection of target polypeptides (e.g., target polypeptides immobilized to a surface, e.g., target polypeptides immobilized in wells of a plate) is contacted with the BV comprising the heterologous membrane-associated protein (e.g., contacted with a solution comprising purified BVs). The assay may then be incubated and washed one or more times to remove non-bound BVs.

In some aspects, an interaction between the heterologous membrane-associated protein and the at least one target polypeptide is identified by detecting a signal at a location on the solid surface that is above a threshold level. The signal detected may be from one or more visualizable components of the BV, as follows.

In some aspects, the membrane-budding agent (e.g., HIV gag protein) further comprises (e.g., is conjugated to) a detectable marker, and detecting an interaction comprises detecting a level of the detectable marker at a location on the solid surface that is above a threshold level. In some aspects, the detectable marker is an enzyme that produces a fluorescent signal in the presence of a substrate. In some aspects, the enzyme is Renilla luciferase (Rluc), and the assay further comprises adding Rluc substrate, thus generating a fluorescent signal at a location on the solid surface at which an interaction has taken place.

In some aspects, the BV comprises a membrane marker, and detecting an interaction comprises detecting a level of the membrane marker at a location on the solid surface that is above a threshold level. In some aspects, the membrane marker is a cholesterol marker. In some aspects, the cholesterol marker is AMPLEX™ Red.

In some aspects, the interaction is a transient interaction.

In some aspects, the interaction is a low-affinity interaction.

In some aspects, the proteins provided in Table 1 and the STM proteins provided in Table 4 are tested for interaction in a protein-protein interaction assay as described above.

In some aspects, the assay described herein may identify the interactions provided in Table 3.

In some aspects, the assay described herein may identify interactions between LRRC15 and TEM1/CD248/endosialin, PLXDC2, PTPRD, SARAF, ASGR1, BMP10, CPM, LDLR, PILRA, and/or PRRG2.

In some aspects, the assay described herein may identify interactions between PD-L1/CD274 and C6orf72, LRTM1, CDHR2, IGF2R, NCR3, and/or SUSD3.

In some aspects, the assay described herein may identify interactions between PVR and CLEC17A and/or PVRL4.

In some aspects, the assay described herein may identify interactions between CD80/B7-1 and BTNL3, CDHR2, GLT8D2, KIAA1467, and/or RNF152.

In some aspects, the assay described herein may identify interactions between CD276/B7-H3 and LRFN1, MXRA5, PVRL1, LRIT2, PLA2R1, and/or SLITRK4. In some aspects, one or more multi-transmembrane receptors (MTMRs), e.g., members of the GPCR superfamily, are tested for interaction in a protein-protein interaction assay as described above.

In some aspects, the proteins provided in Table 5 and the proteins provided in Table 6 are tested for interaction in a protein-protein interaction assay as described above.

In some aspects, the assay described herein may identify the interactions provided in Table 7.

TABLE 5
Query proteins

ADGRB1
LGR4
LGR5

TABLE 6
Library proteins

PD-L1
ICOSLG
DNER
CNTN6
CLPS
EDIL3
IZUMO4
IZUMO1
BTNL3
CD93
CEACAM16
IL-6
LRRC4C
SCARF1
TRIL
CLPS
EDIL3
IZUMO4
CD93
GPR125
IL6R
SCARF1
TRIL

TABLE 7
Interactions between query proteins and library proteins

| Partner A | Partner B |
|---|---|
| ADGRB1 | PD-L1 |
| | ICOSLG |
| | DNER |
| | CNTN6 |
| LGR4 | CLPS |
| | EDIL3 |
| | IZUMO4 |
| | IZUMO1 |
| | BTNL3 |
| | CD93 |
| | CEACAM16 |
| | IL-6 |
| | LRRC4C |
| | SCARF1 |
| | TRIL |
| LGR5 | CLPS |
| | EDIL3 |
| | IZUMO4 |
| | CD93 |
| | GPR125 |
| | IL6R |
| | SCARF1 |
| | TRIL |

In some aspects, the assay described herein may identify interactions between ADGRB1 and PD-L1, ICOSLG, DNER, and/or CNTN6.

In some aspects, the assay described herein may identify interactions between LGR4 and CLPS, EDIL3, IZUMO4, IZUMO1, BTNL3, CD93, CEACAM16, IL-6, LRRC4C, SCARF1, and/or TRIL.

In some aspects, the assay described herein may identify interactions between LGR5 and CLPS, EDIL3, IZUMO4, CD93, GPR125, IL6-R, and/or TRIL.

F. BV-Protein Complexes

In another aspect, the disclosure features a protein complex comprising (a) a BV comprising a heterologous membrane-associated protein and a membrane-budding agent and (b) a target polypeptide, wherein the heterologous membrane-associated protein and the target polypeptide are bound to one another. Exemplary heterologous membrane-associated proteins and target polypeptides are described in Sections IIIA and IIID, respectively. In some aspects, the target polypeptide is immobilized on a surface and the complex is localized on the surface.

### IV. Methods of Identifying a Modulator of a Protein-Protein Interaction

A. Assays for Modulation of Interaction

i. Proteins of Table 1 and Table 2

In some aspects, the disclosure features identifying a modulator of the interaction between a protein of Table 1 and a protein of Table 2, the method comprising: (a) providing a candidate modulator (e.g., a candidate modulator described in Section IV herein); (b) contacting a protein of Table 1 with a protein of Table 2 in the presence or absence of the candidate modulator under conditions permitting the binding of the protein of Table 1 to the protein of Table 2, wherein the protein of Table 1 and the protein of Table 2 are reported to interact in Table 3; and (c) measuring the binding of the protein of Table 1 to the protein of Table 2, wherein an increase or decrease in binding in the presence of the candidate modulator relative to binding in the absence of the candidate modulator identifies the candidate modulator as a modulator of the interaction between the protein of Table 1 and the protein of Table 2.

ii. TEM1 and LRRC15

In some aspects, the disclosure features a method of identifying a modulator of the interaction between LRRC15 and TEM1, the method comprising (a) providing a candidate modulator; (b) contacting LRRC15 with TEM1 in the presence or absence of the candidate modulator under conditions permitting the binding of LRRC15 to TEM1; and (c) measuring the binding of LRRC15 to TEM1, wherein an increase or decrease in binding in the presence of the candidate modulator relative to binding in the absence of the candidate modulator identifies the candidate modulator as a modulator of the interaction between LRRC15 and TEM1.

iii. Proteins of Table 5 and Table 6

In some aspects, the disclosure features identifying a modulator of the interaction between a protein of Table 5 and a protein of Table 6, the method comprising: (a) providing a candidate modulator (e.g., a candidate modulator described in Section IV herein); (b) contacting a protein of Table 5 with a protein of Table 6 in the presence or absence of the candidate modulator under conditions permitting the binding of the protein of Table 5 to the protein of Table 6, wherein the protein of Table 5 and the protein of Table 6 are reported to interact in Table 7; and (c) measuring the binding of the protein of Table 5 to the protein of Table 6, wherein an increase or decrease in binding in the presence of the candidate modulator relative to binding in the absence of the candidate modulator identifies the candidate modulator as a modulator of the interaction between the protein of Table 5 and the protein of Table 6.

iv. PD-L1 and ADGRB1

In some aspects, the disclosure features a method of identifying a modulator of the interaction between PD-L1 and ADGRB1, the method comprising (a) providing a candidate modulator; (b) contacting PD-L1 with ADGRB1 in the presence or absence of the candidate modulator under conditions permitting the binding of PD-L1 to ADGRB1; and (c) measuring the binding of PD-L1 to ADGRB1, wherein an increase or decrease in binding in the presence of the candidate modulator relative to binding in the absence of the candidate modulator identifies the candidate modulator as a modulator of the interaction between PD-L1 and ADGRB1.

v. ICOSLG and ADGRB1

In some aspects, the disclosure features a method of identifying a modulator of the interaction between ICOSLG and ADGRB1, the method comprising (a) providing a candidate modulator; (b) contacting ICOSLG with ADGRB1 in the presence or absence of the candidate modulator under conditions permitting the binding of ICOSLG to ADGRB1; and (c) measuring the binding of ICOSLG to ADGRB1, wherein an increase or decrease in binding in the presence of the candidate modulator relative to binding in the absence of the candidate modulator identifies the candidate modulator as a modulator of the interaction between ICOSLG and ADGRB1.

vi. Assays for Modulation of Interaction

In some aspects, the candidate modulator is provided to a cell (e.g., a mammalian cell); to cell culture media; to conditioned media; to a purified form of a protein of Table 1 (e.g., a form of Protein 1 expressed on a BV) and/or a protein of Table 2; and/or to a purified form of a protein of Table 5 (e.g., a form of Protein 5 expressed on a BV) and/or a protein of Table 6. In some aspects, the candidate modulator is provided at a concentration of at least 0.1 nM, 0.5 nM, 1 nM, 10 nM, 50 nM, 100 nM, 250 nM, 500 nM, 750 nM, 1 μM, 2 μM, 3 μM, 5 μM, or 10 μM. In some aspects, the candidate modulator is provided at a concentration of between 0.1 nM and 10 μM. In some aspects, the candidate modulator is provided in a solution, e.g., in a soluble form.

In some aspects, the candidate modulator is identified as a modulator if the increase in binding is at least 70% (e.g., as measured by surface plasmon resonance, biolayer interferometry, or an enzyme-linked immunosorbent assay (ELISA). In some aspects, the increase in binding is at least 5%, at least 10%, at least 20%, at least 30%, at least 40%, at least 50%, at least 60%, at least 70%, at least 80%, at least 90%, at least 100%, or more than 100% (e.g., 5%-10%, 10%-20%, 20%-30%, 30%-40%, 40%-50%, 50%-60%, 60%-70%, 70%-80%, 80%-90%, 90%-100%, or more than 100%). In some aspects, the increase in binding is at least 70%.

In some aspects, the candidate modulator is identified as a modulator if the decrease in binding is at least 70% (e.g., as measured by surface plasmon resonance, biolayer interferometry, or an enzyme-linked immunosorbent assay (ELISA)). In some aspects, the decrease in binding is at least 5%, at least 10%, at least 20%, at least 30%, at least 40%, at least 50%, at least 60%, at least 70%, at least 80%, at least 90%, or 100% (e.g., 5%-10%, 10%-20%, 20%-30%, 30%-40%, 40%-50%, 50%-60%, 60%-70%, 70%-80%, 80%-90%, or 90%-100%). In some aspects, the decrease in binding is at least 70%. Exemplary methods for identifying modulators of protein-protein interactions, as well as agents that may modulate such interactions, are described below and in PCT/US2020/025471, which is hereby incorporated by reference in its entirety.

Modulation of the interaction between the protein of Table 1 and the protein of Table 2 or between the protein of Table 5 and the protein of Table 6 may be identified as an increase in protein-protein interaction in the presence of the modulator compared to protein-protein interaction in the absence of the modulator, e.g., an increase of 5%, 10%, 15%, 20%, 25%, 30%, 35%, 40%, 45%, 50%, 55%, 60%, 65%, 70%, 80%, 90%, 95%, 100%, or more than 100% (e.g., 5%-10%, 10%-20%, 20%-30%, 30%-40%, 40%-50%, 50%-60%, 60%-70%, 70%-80%, 80%-90%, or 90%-100%) in protein-protein interaction. Alternatively, modulation may be identified as a decrease in protein-protein interaction in the presence of the modulator compared to protein-protein interaction in the absence of the modulator, e.g., an decrease of 5%, 10%, 15%, 20%, 25%, 30%, 35%, 40%, 45%, 50%, 55%, 60%, 65%, 70%, 80%, 90%, 95%, or 100% (e.g., 10%-20%, 20%-30%, 30%-40%, 40%-50%, 50%-60%, 60%-70%, 70%-80%, 80%-90%, or 90%-100%) in protein-protein interaction. The assay for protein-protein interaction may be, e.g., a surface plasmon resonance (SPR) assay, a biolayer interferometry (BLI) assay, an enzyme-linked immunosorbent assay (ELISA), an extracellular interaction assay, or a cell surface interaction assay.

SPR Assays for Modulation of Protein-Protein Interaction

In some aspects, the assay for protein-protein interaction is a surface plasmon resonance (SPR) assay. In some aspects, modulation of the binding of the protein of Table 1 to the protein of Table 2 or of the protein of Table 5 to the protein of Table 6 is measured as a difference in SPR signal response units (RU) in the presence compared to the absence of the modulator.

BLI Assays for Modulation of Protein-Protein Interaction

In some aspects, the assay for protein-protein interaction is a biolayer interferometry (BLI) assay. In some aspects, the BLI assay is performed using isolated extracellular domains (ECDs). In some aspects, modulation of the binding of the protein of Table 1 to the protein of Table 2 or of the protein of Table 5 to the protein of Table 6 is measured as a difference in wavelength shift (Δλ) measured at a biosensor tip in the presence compared to the absence of the modulator.

ELISA for Modulation of Protein-Protein Interaction

In some aspects, the assay for protein-protein interaction is an enzyme-linked immunosorbent assay (ELISA). In some aspects, a first protein is bound to a plate (e.g., directly bound to a plate or bound to a plate via an affinity tag recognized by an antibody bound to a plate) and a second protein is provided in a soluble form, e.g., as an isolated ECD. An interaction between the first protein and the second protein may be detected by providing an antibody that binds to the second protein or to an affinity tag thereof, wherein the antibody can be detected, e.g., visualized, in an assay for presence of the antibody.

Other Assays for Modulation of Protein-Protein Interaction

In some aspects, the assay is an extracellular interaction assay, e.g., as described in PCT/US2020/025471, which is incorporated herein by reference in its entirety. In some aspects, the assay is a cell surface interaction assay, e.g., as described in PCT/US2020/025471. In some aspects, the assay is an isothermal titration calorimetry (ITC) assay, an assay comprising immunoprecipitation, or an assay comprising an ALPHASCREEN™ technology.

B. Assays for Changes in Downstream Activity

i. Proteins of Table 1 and Table 2

In some aspects, the disclosure features a method of identifying a modulator of a downstream activity of a protein of Table 1, the method comprising: (a) providing a candidate modulator (e.g., a candidate modulator described in Section IV herein); (b) contacting the protein of Table 1 with a protein of Table 2 in the presence or absence of the candidate modulator under conditions permitting the binding of the protein of Table 1 to the protein of Table 2, wherein the protein of Table 1 and the protein of Table 2 are reported to interact in Table 3; and (c) measuring a downstream activity of the protein of Table 1, wherein a change in the downstream activity in the presence of the candidate modulator relative to the downstream activity in the absence of the candidate modulator identifies the candidate modulator as a modulator of the downstream activity of the protein of Table 1.

In some aspects, the disclosure features a method of identifying a modulator of a downstream activity of a protein of Table 2, the method comprising: (a) providing a candidate modulator (e.g., a candidate modulator described in Section IV herein); (b) contacting the protein of Table 2 with a protein of Table 1 in the presence or absence of the candidate modulator under conditions permitting the binding of the protein of Table 2 to the protein of Table 1, wherein the protein of Table 1 and the protein of Table 2 are reported to interact in Table 3; and (c) measuring a downstream activity of the protein of Table 2, wherein a change in the downstream activity in the presence of the candidate modulator relative to the downstream activity in the absence of the candidate modulator identifies the candidate modulator as a modulator of the downstream activity of the protein of Table 2.

ii. TEM1 and LRRC15

In some aspects, the disclosure features a method of identifying a modulator of a downstream activity of LRRC15, the method comprising (a) providing a candidate modulator; (b) contacting LRRC15 with TEM1 in the presence or absence of the candidate modulator under conditions permitting the binding of LRRC15 to TEM1; and (c) measuring a downstream activity of LRRC15, wherein a change in the downstream activity in the presence of the candidate modulator relative to the downstream activity in the absence of the candidate modulator identifies the candidate modulator as a modulator of the downstream activity of LRRC15.

In some aspects, the disclosure features a method of identifying a modulator of a downstream activity of TEM1, the method comprising (a) providing a candidate modulator; (b) contacting TEM1 with LRRC15 in the presence or absence of the candidate modulator under conditions permitting the binding of TEM1 to LRRC15; and (c) measuring a downstream activity of TEM1, wherein a change in the downstream activity in the presence of the candidate modulator relative to the downstream activity in the absence of the candidate modulator identifies the candidate modulator as a modulator of the downstream activity of TEM1. In some aspects, the increase or decrease in binding is at least 70%, as measured by surface plasmon resonance, biolayer interferometry, or ELISA.

iii. Proteins of Table 5 and Table 6

In some aspects, the disclosure features a method of identifying a modulator of a downstream activity of a protein of Table 5, the method comprising: (a) providing a candidate modulator (e.g., a candidate modulator described in Section IV herein); (b) contacting the protein of Table 5 with a protein of Table 6 in the presence or absence of the candidate modulator under conditions permitting the binding of the protein of Table 5 to the protein of Table 6, wherein the protein of Table 5 and the protein of Table 6 are reported to interact in Table 7; and (c) measuring a downstream activity of the protein of Table 5, wherein a change in the downstream activity in the presence of the candidate modulator relative to the downstream activity in the absence of the candidate modulator identifies the candidate modulator as a modulator of the downstream activity of the protein of Table 5.

In some aspects, the disclosure features a method of identifying a modulator of a downstream activity of a protein of Table 6, the method comprising: (a) providing a candidate modulator (e.g., a candidate modulator described in Section IV herein); (b) contacting the protein of Table 6 with a protein of Table 5 in the presence or absence of the candidate modulator under conditions permitting the binding of the protein of Table 6 to the protein of Table 5, wherein the protein of Table 5 and the protein of Table 6 are reported to interact in Table 7; and (c) measuring a downstream activity of the protein of Table 6, wherein a change in the downstream activity in the presence of the candidate modulator relative to the downstream activity in the absence of the candidate modulator identifies the candidate modulator as a modulator of the downstream activity of the protein of Table 6.

iv. PD-L1 and ADGRB1

In some aspects, the disclosure features a method of identifying a modulator of a downstream activity of PD-L1, the method comprising (a) providing a candidate modulator; (b) contacting PD-L1 with ADGRB1 in the presence or absence of the candidate modulator under conditions permitting the binding of PD-L1 to ADGRB1; and (c) measuring a downstream activity of PD-L1, wherein a change in the downstream activity in the presence of the candidate modulator relative to the downstream activity in the absence of the candidate modulator identifies the candidate modulator as a modulator of the downstream activity of PD-1.

In some aspects, the disclosure features a method of identifying a modulator of a downstream activity of ADGRB1, the method comprising (a) providing a candidate modulator; (b) contacting ADGRB1 with PD-L1 in the presence or absence of the candidate modulator under conditions permitting the binding of ADGRB1 to PD-L1; and (c) measuring a downstream activity of ADGRB1, wherein a change in the downstream activity in the presence of the candidate modulator relative to the downstream activity in the absence of the candidate modulator identifies the candidate modulator as a modulator of the downstream activity of ADGRB1. In some aspects, the increase or decrease in binding is at least 70%, as measured by surface plasmon resonance, biolayer interferometry, or ELISA.

v. ICOSLG and ADGRB1

In some aspects, the disclosure features a method of identifying a modulator of a downstream activity of ICOSLG, the method comprising (a) providing a candidate modulator; (b) contacting ICOSLG with ADGRB1 in the presence or absence of the candidate modulator under conditions permitting the binding of ICOSLG to ADGRB1; and (c) measuring a downstream activity of ICOSLG, wherein a change in the downstream activity in the presence of the candidate modulator relative to the downstream activity in the absence of the candidate modulator identifies the candidate modulator as a modulator of the downstream activity of ICOSLG.

In some aspects, the disclosure features a method of identifying a modulator of a downstream activity of ADGRB1, the method comprising (a) providing a candidate modulator; (b) contacting ADGRB1 with ICOSLG in the presence or absence of the candidate modulator under conditions permitting the binding of ADGRB1 to ICOSLG; and (c) measuring a downstream activity of ADGRB1, wherein a change in the downstream activity in the presence of the candidate modulator relative to the downstream activity in the absence of the candidate modulator identifies the candidate modulator as a modulator of the downstream activity of ADGRB1. In some aspects, the increase or decrease in binding is at least 70%, as measured by surface plasmon resonance, biolayer interferometry, or ELISA.

vi. Assays for Changes in Downstream Activity

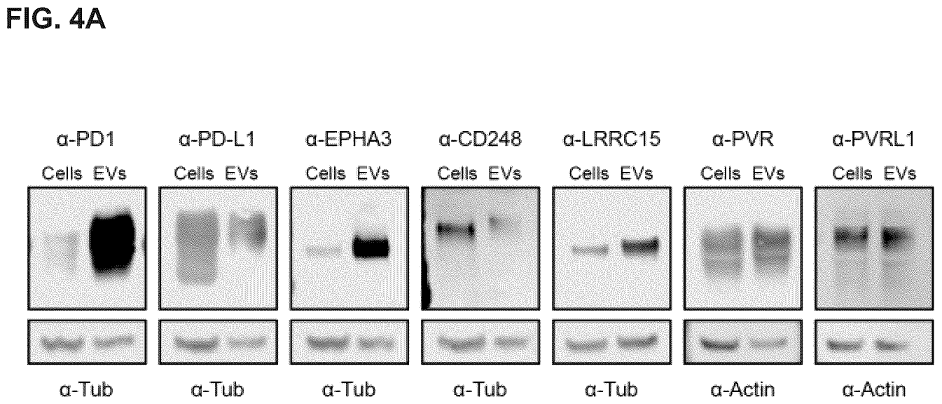

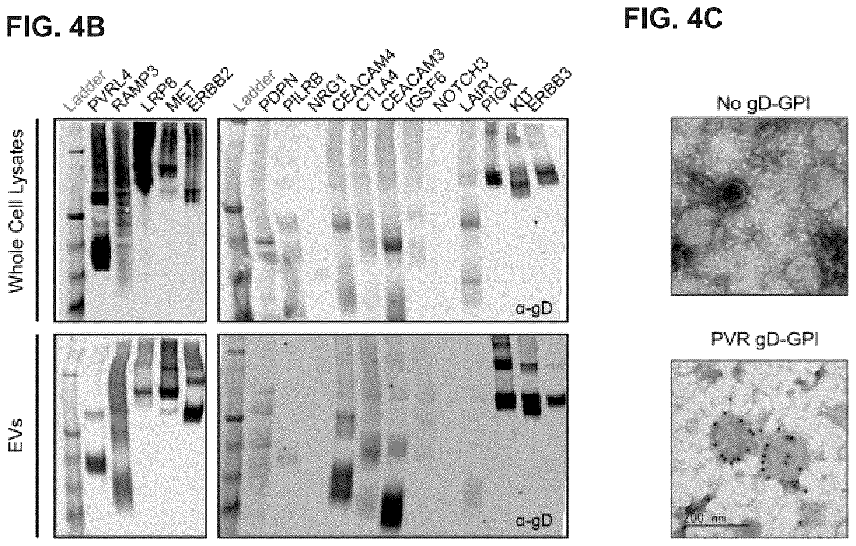

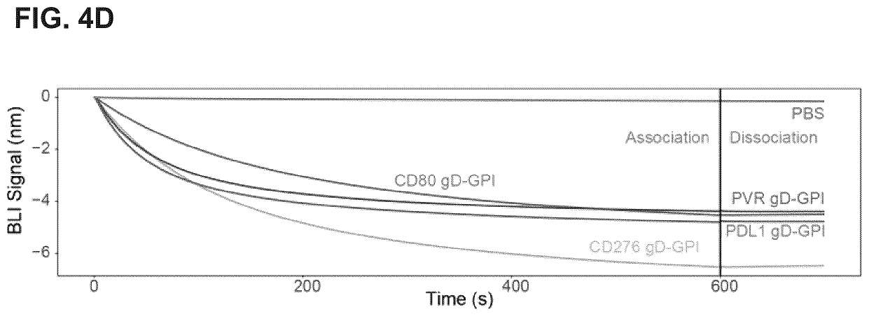

In some aspects, the candidate modulator is provided at a concentration of at least 0.1 nM, 0.5 nM, 1 nM, 10 nM, 50 nM, 100 nM, 250 nM, 500 nM, 750 nM, 1 μM, 2 μM, 3 μM, 5 μM, or 10 μM. In some aspects, the candidate modulator is provided at a concentration of between 0.1 nM and 10 μM. In some aspects, the candidate modulator is provided at a range of concentrations, e.g., as in FIG. 4F. In some aspects, the candidate modulator is provided is provided in a solution, e.g., in a soluble form. In some aspects, the candidate modulator is provided to an organism comprising the protein of Table 1 and the protein of Table 2, to a tissue comprising the protein of Table 1 and the protein of Table 2, to a cell (e.g., a mammalian cell), to cell culture media, to conditioned media, and/or to a purified form of a protein of Table 1 and/or a protein of Table 2. In some aspects, the candidate modulator is provided to an organism comprising the protein of Table 5 and the protein of Table 6, to a tissue comprising the protein of Table 5 and the protein of Table 6, to a cell (e.g., a mammalian cell), to cell culture media, to conditioned media, and/or to a purified form of a protein of Table 5 and/or a protein of Table 6.

In some aspects, the modulator is an activator of the downstream activity of the protein of Table 1 or Table 2. In some aspects, the candidate modulator is identified as a modulator if the increase in the downstream activity of the protein of Table 1 or the protein of Table 2 is at least 30%. In some aspects, the modulator is an activator of the downstream activity of the protein of Table 5 or Table 6. In some aspects, the candidate modulator is identified as a modulator if the increase in the downstream activity of the protein of Table 5 or the protein of Table 6 is at least 30%. In some aspects, the increase in the downstream activity is at least 5%, at least 10%, at least 20%, at least 30%, at least 40%, at least 50%, at least 60%, at least 70%, at least 80%, at least 90%, at least 100%, or more than 100% (e.g., at least 5%-10%, 10%-20%, 20%-30%, 30%-40%, 40%-50%, 50%-60%, 60%-70%, 70%-80%, 80%-90%, 90%-100%, or more than 100%). In some aspects, the increase in the downstream activity is at least 30%. In some aspects, the change in the downstream activity is an increase in the amount, strength, or duration of the downstream activity.

In some aspects, the modulator is an inhibitor of the downstream activity of the protein of Table 1 or Table 2. In some aspects, the candidate modulator is identified as a modulator if the decrease in the downstream activity of the protein of Table 1 or the protein of Table 2 is at least 30%. In some aspects, the modulator is an inhibitor of the downstream activity of the protein of Table 5 or Table 6. In some aspects, the candidate modulator is identified as a modulator if the decrease in the downstream activity of the protein of Table 5 or the protein of Table 6 is at least 30%. In some aspects, the decrease in the downstream activity is at least 5%, at least 10%, at least 20%, at least 30%, at least 40%, at least 50%, at least 60%, at least 70%, at least 80%, at least 90%, or 100% (e.g., at least 5%-10%, 10%-20%, 20%-30%, 30%-40%, 40%-50%, 50%-60%, 60%-70%, 70%-80%, 80%-90%, or 90%-100%). In some aspects, the decrease in downstream activity is at least 30%. In some aspects, the change in the downstream activity is a decrease in the amount, strength, or duration of the downstream activity.

In some aspects, the downstream activity of the protein of Table 1 or the protein of Table 2 is assessed in one or more assays. In some aspects, the downstream activity of the protein of Table 5 or the protein of Table 6 is assessed in one or more assays.

In some aspects, the downstream activity is an activity relating to the development or progression of a disease, e.g., a cancer.

In some aspects, the downstream activity is tumor growth. In some aspects, the protein of Table 1 is LRRC15, the protein of Table 2 is TEM1, and the downstream activity is tumor growth. In some aspects, tumor growth is decreased by at least 10%, at least 20%, at least 30%, at least 40%, at least 50%, at least 60%, at least 70%, at least 80%, at least 90%, or 100% (e.g., at least 10%-20%, 20%-30%, 30%-40%, 40%-50%, 50%-60%, 60%-70%, 70%-80%, 80%-90%, or 90%-100%) in the presence of the modulator, as measured in a tumor growth assay. In some aspects, tumor growth is decreased by at least 20% in the presence of the modulator, as measured in a tumor growth assay.

In some aspects, the protein of Table 5 is ADGRB, the protein of Table 2 is PD-L1, and the downstream activity is tumor growth. In some aspects, tumor growth is decreased by at least 10%, at least 20%, at least 30%, at least 40%, at least 50%, at least 60%, at least 70%, at least 80%, at least 90%, or 100% (e.g., at least 10%-20%, 20%-30%, 30%-40%, 40%-50%, 50%-60%, 60%-70%, 70%-80%, 80%-90%, or 90%-100%) in the presence of the modulator, as measured in a tumor growth assay. In some aspects, tumor growth is decreased by at least 20% in the presence of the modulator, as measured in a tumor growth assay.

In some aspects, the protein of Table 5 is ADGRB, the protein of Table 2 is PD-L1, and the downstream activity is engulfment of bacterial cells or apoptotic cells. In some aspects, the rate of engulfment is increased by at least 10%, at least 20%, at least 30%, at least 40%, at least 50%, at least 60%, at least 70%, at least 80%, at least 90%, or 100% (e.g., at least 10%-20%, 20%-30%, 30%-40%, 40%-50%, 50%-60%, 60%-70%, 70%-80%, 80%-90%, or 90%-100%) in the presence of the modulator.

In some aspects, the protein of Table 5 is ADGRB, the protein of Table 2 is ICOSLG, and the downstream activity is T cell activation. In some aspects, T cell activation is increased by at least 10%, at least 20%, at least 30%, at least 40%, at least 50%, at least 60%, at least 70%, at least 80%, at least 90%, or 100% (e.g., at least 10%-20%, 20%-30%, 30%-40%, 40%-50%, 50%-60%, 60%-70%, 70%-80%, 80%-90%, or 90%-100%). In some aspects, T cell activation is increased by at least 20% in the presence of the modulator.

C. Small Molecules

In some aspects, the modulator or candidate modulator is a small molecule. Small molecules are molecules other than binding polypeptides or antibodies as defined herein that may bind, preferably specifically, to a protein of Table 1 and/or a protein of Table 2 or to a protein of Table 5 and/or a protein of Table 6. Binding small molecules may be identified and chemically synthesized using known methodology (see, e.g., PCT Publication Nos. WO00/00823 and WO00/39585). Binding small molecules are usually less than about 2000 daltons in size (e.g., less than about 2000, 1500, 750, 500, 250 or 200 daltons in size), wherein such organic small molecules that are capable of binding, preferably specifically, to a polypeptide as described herein may be identified without undue experimentation using well known techniques. In this regard, it is noted that techniques for screening small molecule libraries for molecules that are capable of binding to a polypeptide target are well known in the art (see, e.g., PCT Publication Nos. WO00/00823 and WO00/39585). Binding small molecules may be, for example, aldehydes, ketones, oximes, hydrazones, semicarbazones, carbazides, primary amines, secondary amines, tertiary amines, N-substituted hydrazines, hydrazides, alcohols, ethers, thiols, thioethers, disulfides, carboxylic acids, esters, amides, ureas, carbamates, carbonates, ketals, thioketals, acetals, thioacetals, aryl halides, aryl sulfonates, alkyl halides, alkyl sulfonates, aromatic compounds, heterocyclic compounds, anilines, alkenes, alkynes, diols, amino alcohols, oxazolidines, oxazolines, thiazolidines, thiazolines, enamines, sulfonamides, epoxides, aziridines, isocyanates, sulfonyl chlorides, diazo compounds, acid chlorides, or the like.

In some aspects, the binding of a protein of Table 1 and a protein of Table 2 is decreased (e.g., decreased by 5%, 10%, 20%, 30%, 40%, 50%, 60%, 70%, 80%, 90%, or 100% (e.g., 5%-10%, 10%-20%, 20%-30%, 30%-40%, 40%-50%, 50%-60%, 60%-70%, 70%-80%, 80%-90%, or 90%-100%)) in the presence of the small molecule. In some aspects, the binding of a protein of Table 1 and a protein of Table 2 is increased (e.g., increased by 5%, 10%, 20%, 30%, 40%, 50%, 60%, 70%, 80%, 90%, 100%, or more than 100% (e.g., 5%-10%, 10%-20%, 20%-30%, 30%-40%, 40%-50%, 50%-60%, 60%-70%, 70%-80%, 80%-90%, 90%-100%, or more than 100%)) in the presence of the small molecule. In some aspects, a downstream activity of the protein of Table 1 and/or the protein of Table 2 is decreased (e.g., decreased by 5%, 10%, 20%, 30%, 40%, 50%, 60%, 70%, 80%, 90%, or 100% (e.g., 5%-10%, 10%-20%, 20%-30%, 30%-40%, 40%-50%, 50%-60%, 60%-70%, 70%-80%, 80%-90%, or 90%-100%)) in the presence of the small molecule. In some aspects, a downstream activity of the protein of Table 1 and/or the protein of Table 2 is increased (e.g., increased by 5%, 10%, 20%, 30%, 40%, 50%, 60%, 70%, 80%, 90%, 100%, or more than 100% (e.g., 5%-10%, 10%-20%, 20%-30%, 30%-40%, 40%-50%, 50%-60%, 60%-70%, 70%-80%, 80%-90%, 90%-100%, or more than 100%)) in the presence of the small molecule.

In some aspects, the binding of a protein of Table 5 and a protein of Table 6 is decreased (e.g., decreased by 5%, 10%, 20%, 30%, 40%, 50%, 60%, 70%, 80%, 90%, or 100% (e.g., 5%-10%, 10%-20%, 20%-30%, 30%-40%, 40%-50%, 50%-60%, 60%-70%, 70%-80%, 80%-90%, or 90%-100%)) in the presence of the small molecule. In some aspects, the binding of a protein of Table 5 and a protein of Table 6 is increased (e.g., increased by 5%, 10%, 20%, 30%, 40%, 50%, 60%, 70%, 80%, 90%, 100%, or more than 100% (e.g., 5%-10%, 10%-20%, 20%-30%, 30%-40%, 40%-50%, 50%-60%, 60%-70%, 70%-80%, 80%-90%, 90%-100%, or more than 100%)) in the presence of the small molecule. In some aspects, a downstream activity of the protein of Table 5 and/or the protein of Table 6 is decreased (e.g., decreased by 5%, 10%, 20%, 30%, 40%, 50%, 60%, 70%, 80%, 90%, or 100% (e.g., 5%-10%, 10%-20%, 20%-30%, 30%-40%, 40%-50%, 50%-60%, 60%-70%, 70%-80%, 80%-90%, or 90%-100%)) in the presence of the small molecule. In some aspects, a downstream activity of the protein of Table 5 and/or the protein of Table 6 is increased (e.g., increased by 5%, 10%, 20%, 30%, 40%, 50%, 60%, 70%, 80%, 90%, 100%, or more than 100% (e.g., 5%-10%, 10%-20%, 20%-30%, 30%-40%, 40%-50%, 50%-60%, 60%-70%, 70%-80%, 80%-90%, 90%-100%, or more than 100%)) in the presence of the small molecule.

D. Antibodies and Antigen-Binding Fragments

In some aspects, the modulator or candidate modulator is an antibody or an antigen-binding fragment thereof binding a protein of Table 1 and/or a protein of Table 2. In some aspects, the antigen-binding fragment is a bis-Fab, an Fv, a Fab, a Fab′-SH, a F(ab′)₂, a diabody, a linear antibody, an scFv, an ScFab, a VH domain, or a VHH domain. In some aspects, the antibody or antigen-binding fragment thereof binds the protein of Table 1. In other aspects, the antibody or antigen-binding fragment thereof binds the protein of Table 2.

In some aspects, the binding of a protein of Table 1 and a protein of Table 2 is decreased (e.g., decreased by 5%, 10%, 20%, 30%, 40%, 50%, 60%, 70%, 80%, 90%, or 100% (e.g., 5%-10%, 10%-20%, 20%-30%, 30%-40%, 40%-50%, 50%-60%, 60%-70%, 70%-80%, 80%-90%, or 90%-100%)) in the presence of the antibody or antigen-binding fragment. In some aspects, the binding of a protein of Table 1 and a protein of Table 2 is increased (e.g., increased by 5%, 10%, 20%, 30%, 40%, 50%, 60%, 70%, 80%, 90%, 100%, or more than 100% (e.g., 5%-10%, 10%-20%, 20%-30%, 30%-40%, 40%-50%, 50%-60%, 60%-70%, 70%-80%, 80%-90%, 90%-100%, or more than 100%)) in the presence of the antibody or antigen-binding fragment. In some aspects, a downstream activity (e.g., a downstream activity described in Section IIIB herein, e.g., CAF contractility, immune checkpoint inhibition, suppression of cell proliferation, modulation of target phosphorylation, inhibition of cell migration, suppression of tumor formation, suppression of cell invasion, macrophage polarization, regulation of phagocytosis, osteoclast differentiation, activation of a signaling pathway, or formation of filopodia) of the protein of Table 1 and/or the protein of Table 2 is decreased (e.g., decreased by 5%, 10%, 20%, 30%, 40%, 50%, 60%, 70%, 80%, 90%, or 100% (e.g., 5%-10%, 10%-20%, 20%-30%, 30%-40%, 40%-50%, 50%-60%, 60%-70%, 70%-80%, 80%-90%, or 90%-100%)) in the presence of the antibody or antigen-binding fragment. In some aspects, a downstream activity (e.g., a downstream activity described in Section IIIB herein) of the protein of Table 1 and/or the protein of Table 2 is increased (e.g., increased by 5%, 10%, 20%, 30%, 40%, 50%, 60%, 70%, 80%, 90%, 100%, or more than 100% (e.g., 5%-10%, 10%-20%, 20%-30%, 30%-40%, 40%-50%, 50%-60%, 60%-70%, 70%-80%, 80%-90%, 90%-100%, or more than 100%)) in the presence of the antibody or antigen-binding fragment.

In some aspects, the modulator or candidate modulator is an antibody or an antigen-binding fragment thereof binding a protein of Table 5 and/or a protein of Table 6. In some aspects, the antigen-binding fragment is a bis-Fab, an Fv, a Fab, a Fab′-SH, a F(ab′)₂, a diabody, a linear antibody, an scFv, an ScFab, a VH domain, or a VHH domain. In some aspects, the antibody or antigen-binding fragment thereof binds the protein of Table 5. In other aspects, the antibody or antigen-binding fragment thereof binds the protein of Table 6.

In some aspects, the binding of a protein of Table 5 and a protein of Table 6 is decreased (e.g., decreased by 5%, 10%, 20%, 30%, 40%, 50%, 60%, 70%, 80%, 90%, or 100% (e.g., 5%-10%, 10%-20%, 20%-30%, 30%-40%, 40%-50%, 50%-60%, 60%-70%, 70%-80%, 80%-90%, or 90%-100%)) in the presence of the antibody or antigen-binding fragment. In some aspects, the binding of a protein of Table 5 and a protein of Table 6 is increased (e.g., increased by 5%, 10%, 20%, 30%, 40%, 50%, 60%, 70%, 80%, 90%, 100%, or more than 100% (e.g., 5%-10%, 10%-20%, 20%-30%, 30%-40%, 40%-50%, 50%-60%, 60%-70%, 70%-80%, 80%-90%, 90%-100%, or more than 100%)) in the presence of the antibody or antigen-binding fragment. In some aspects, a downstream activity (e.g., a downstream activity described in Section IIIB herein, e.g., CAF contractility, immune checkpoint inhibition, suppression of cell proliferation, modulation of target phosphorylation, inhibition of cell migration, suppression of tumor formation, suppression of cell invasion, macrophage polarization, regulation of phagocytosis, osteoclast differentiation, activation of a signaling pathway, or formation of filopodia) of the protein of Table 5 and/or the protein of Table 6 is decreased (e.g., decreased by 5%, 10%, 20%, 30%, 40%, 50%, 60%, 70%, 80%, 90%, or 100% (e.g., 5%-10%, 10%-20%, 20%-30%, 30%-40%, 40%-50%, 50%-60%, 60%-70%, 70%-80%, 80%-90%, or 90%-100%)) in the presence of the antibody or antigen-binding fragment. In some aspects, a downstream activity (e.g., a downstream activity described in Section IIIB herein) of the protein of Table 5 and/or the protein of Table 6 is increased (e.g., increased by 5%, 10%, 20%, 30%, 40%, 50%, 60%, 70%, 80%, 90%, 100%, or more than 100% (e.g., 5%-10%, 10%-20%, 20%-30%, 30%-40%, 40%-50%, 50%-60%, 60%-70%, 70%-80%, 80%-90%, 90%-100%, or more than 100%)) in the presence of the antibody or antigen-binding fragment.

E. Peptides

In some aspects, the modulator or candidate modulator is a peptide that binds to a protein of Table 1 and/or a protein of Table 2. The peptide may be the peptide may be naturally occurring or may be engineered. In some aspects, the peptide is a fragment of the protein of Table 1, the protein of Table 2, or another protein that binds to the protein of Table 1 or the protein of Table 2. The peptide may bind the binding partner with equal, less, or greater affinity than the full-length protein. In some aspects, the peptide performs all functions of the full-length protein. In other aspects, the peptide does not perform all functions of the full-length protein.

In some aspects, the binding of a protein of Table 1 and a protein of Table 2 is decreased (e.g., decreased by 5%, 10%, 20%, 30%, 40%, 50%, 60%, 70%, 80%, 90%, or 100% (e.g., 5%-10%, 10%-20%, 20%-30%, 30%-40%, 40%-50%, 50%-60%, 60%-70%, 70%-80%, 80%-90%, or 90%-100%)) in the presence of the peptide. In some aspects, the binding of a protein of Table 1 and a protein of Table 2 is increased (e.g., increased by 5%, 10%, 20%, 30%, 40%, 50%, 60%, 70%, 80%, 90%, 100%, or more than 100% (e.g., 5%-10%, 10%-20%, 20%-30%, 30%-40%, 40%-50%, 50%-60%, 60%-70%, 70%-80%, 80%-90%, 90%-100%, or more than 100%)) in the presence of the peptide. In some aspects, a downstream activity of the protein of Table 1 and/or the protein of Table 2 is decreased (e.g., decreased by 5%, 10%, 20%, 30%, 40%, 50%, 60%, 70%, 80%, 90%, or 100% (e.g., 5%-10%, 10%-20%, 20%-30%, 30%-40%, 40%-50%, 50%-60%, 60%-70%, 70%-80%, 80%-90%, or 90%-100%)) in the presence of the peptide. In some aspects, a downstream activity of the protein of Table 1 and/or the protein of Table 2 is increased (e.g., increased by 5%, 10%, 20%, 30%, 40%, 50%, 60%, 70%, 80%, 90%, 100%, or more than 100% (e.g., 5%-10%, 10%-20%, 20%-30%, 30%-40%, 40%-50%, 50%-60%, 60%-70%, 70%-80%, 80%-90%, 90%-100%, or more than 100%)) in the presence of the peptide.

In some aspects, the modulator or candidate modulator is a peptide that binds to a protein of Table 5 and/or a protein of Table 6. The peptide may be the peptide may be naturally occurring or may be engineered. In some aspects, the peptide is a fragment of the protein of Table 5, the protein of Table 6, or another protein that binds to the protein of Table 5 or the protein of Table 6. The peptide may bind the binding partner with equal, less, or greater affinity than the full-length protein. In some aspects, the peptide performs all functions of the full-length protein. In other aspects, the peptide does not perform all functions of the full-length protein.

In some aspects, the binding of a protein of Table 5 and a protein of Table 6 is decreased (e.g., decreased by 5%, 10%, 20%, 30%, 40%, 50%, 60%, 70%, 80%, 90%, or 100% (e.g., 5%-10%, 10%-20%, 20%-30%, 30%-40%, 40%-50%, 50%-60%, 60%-70%, 70%-80%, 80%-90%, or 90%-100%)) in the presence of the peptide. In some aspects, the binding of a protein of Table 5 and a protein of Table 6 is increased (e.g., increased by 5%, 10%, 20%, 30%, 40%, 50%, 60%, 70%, 80%, 90%, 100%, or more than 100% (e.g., 5%-10%, 10%-20%, 20%-30%, 30%-40%, 40%-50%, 50%-60%, 60%-70%, 70%-80%, 80%-90%, 90%-100%, or more than 100%)) in the presence of the peptide. In some aspects, a downstream activity of the protein of Table 5 and/or the protein of Table 6 is decreased (e.g., decreased by 5%, 10%, 20%, 30%, 40%, 50%, 60%, 70%, 80%, 90%, or 100% (e.g., 5%-10%, 10%-20%, 20%-30%, 30%-40%, 40%-50%, 50%-60%, 60%-70%, 70%-80%, 80%-90%, or 90%-100%)) in the presence of the peptide. In some aspects, a downstream activity of the protein of Table 5 and/or the protein of Table 6 is increased (e.g., increased by 5%, 10%, 20%, 30%, 40%, 50%, 60%, 70%, 80%, 90%, 100%, or more than 100% (e.g., 5%-10%, 10%-20%, 20%-30%, 30%-40%, 40%-50%, 50%-60%, 60%-70%, 70%-80%, 80%-90%, 90%-100%, or more than 100%)) in the presence of the peptide.

F. Mimics

In some aspects, the modulator or candidate modulator is a mimic, e.g., a molecular mimic, that binds to a protein of Table 1 and/or a protein of Table 2. The mimic may be a molecular mimic of the protein of Table 1, the protein of Table 2, or another protein that binds to the protein of Table 1 or the protein of Table 2. In some aspects, the mimic may perform all functions of the mimicked polypeptide. In other aspects, the mimic does not perform all functions of the mimicked polypeptide.

In some aspects, the binding of a protein of Table 1 and a protein of Table 2 is decreased (e.g., decreased by 5%, 10%, 20%, 30%, 40%, 50%, 60%, 70%, 80%, 90%, or 100% (e.g., 5%-10%, 10%-20%, 20%-30%, 30%-40%, 40%-50%, 50%-60%, 60%-70%, 70%-80%, 80%-90%, or 90%-100%)) in the presence of the mimic. In some aspects, the binding of a protein of Table 1 and a protein of Table 2 is increased (e.g., increased by 5%, 10%, 20%, 30%, 40%, 50%, 60%, 70%, 80%, 90%, 100%, or more than 100% (e.g., 5%-10%, 10%-20%, 20%-30%, 30%-40%, 40%-50%, 50%-60%, 60%-70%, 70%-80%, 80%-90%, 90%-100%, or more than 100%)) in the presence of the mimic. In some aspects, a downstream activity of the protein of Table 1 and/or the protein of Table 2 is decreased (e.g., decreased by 5%, 10%, 20%, 30%, 40%, 50%, 60%, 70%, 80%, 90%, or 100% (e.g., 5%-10%, 10%-20%, 20%-30%, 30%-40%, 40%-50%, 50%-60%, 60%-70%, 70%-80%, 80%-90%, or 90%-100%)) in the presence of the mimic. In some aspects, a downstream activity of the protein of Table 1 and/or the protein of Table 2 is increased (e.g., increased by 5%, 10%, 20%, 30%, 40%, 50%, 60%, 70%, 80%, 90%, 100%, or more than 100% (e.g., 5%-10%, 10%-20%, 20%-30%, 30%-40%, 40%-50%, 50%-60%, 60%-70%, 70%-80%, 80%-90%, 90%-100%, or more than 100%)) in the presence of the mimic.

In some aspects, the modulator or candidate modulator is a mimic, e.g., a molecular mimic, that binds to a protein of Table 5 and/or a protein of Table 6. The mimic may be a molecular mimic of the protein of Table 5, the protein of Table 6, or another protein that binds to the protein of Table 5 or the protein of Table 6. In some aspects, the mimic may perform all functions of the mimicked polypeptide. In other aspects, the mimic does not perform all functions of the mimicked polypeptide.

In some aspects, the binding of a protein of Table 5 and a protein of Table 6 is decreased (e.g., decreased by 5%, 10%, 20%, 30%, 40%, 50%, 60%, 70%, 80%, 90%, or 100% (e.g., 5%-10%, 10%-20%, 20%-30%, 30%-40%, 40%-50%, 50%-60%, 60%-70%, 70%-80%, 80%-90%, or 90%-100%)) in the presence of the mimic. In some aspects, the binding of a protein of Table 5 and a protein of Table 6 is increased (e.g., increased by 5%, 10%, 20%, 30%, 40%, 50%, 60%, 70%, 80%, 90%, 100%, or more than 100% (e.g., 5%-10%, 10%-20%, 20%-30%, 30%-40%, 40%-50%, 50%-60%, 60%-70%, 70%-80%, 80%-90%, 90%-100%, or more than 100%)) in the presence of the mimic. In some aspects, a downstream activity of the protein of Table 5 and/or the protein of Table 6 is decreased (e.g., decreased by 5%, 10%, 20%, 30%, 40%, 50%, 60%, 70%, 80%, 90%, or 100% (e.g., 5%-10%, 10%-20%, 20%-30%, 30%-40%, 40%-50%, 50%-60%, 60%-70%, 70%-80%, 80%-90%, or 90%-100%)) in the presence of the mimic. In some aspects, a downstream activity of the protein of Table 5 and/or the protein of Table 6 is increased (e.g., increased by 5%, 10%, 20%, 30%, 40%, 50%, 60%, 70%, 80%, 90%, 100%, or more than 100% (e.g., 5%-10%, 10%-20%, 20%-30%, 30%-40%, 40%-50%, 50%-60%, 60%-70%, 70%-80%, 80%-90%, 90%-100%, or more than 100%)) in the presence of the mimic.

### V. Methods of Treatment Comprising Modulators of Identified Protein-Protein Interactions

In some aspects, a modulator of a protein-protein interaction described herein is used to treat or delay progression of a pathological state, disease, disorder, or condition, e.g., a cancer.

In some aspects, the modulator increases or decreases the amount, strength, or duration of a downstream activity of the protein-protein interaction, e.g., tumor formation or tumor growth, in an individual to whom the modulator has been administered.

A. Cancers

In some aspects, a modulator of a protein-protein interaction described herein (e.g., a modulator of the interaction between LRRC15 and TEM1; a modulator of an interaction between PD-L1 and ADGRB1; or a modulator of an interaction between ADGRB1 and ICOSLG), e.g., a small molecule, an antibody, an antigen-binding fragment, a peptide, a mimic, an antisense oligonucleotide, or an siRNA, is used to treat or delay progression of a cancer in a subject in need thereof. In some aspects, the subject is a human. The cancer may be a solid tumor cancer or a non-solid tumor cancer. Solid cancer tumors include, but are not limited to a bladder cancer, a melanoma, a breast cancer, a colorectal cancer, a lung cancer, a head and neck cancer, a kidney cancer, an ovarian cancer, a pancreatic cancer, or a prostate cancer, or metastatic forms thereof. In some aspects, the cancer is a bladder cancer. Further aspects of bladder cancer include urothelial carcinoma, muscle invasive bladder cancer (MIBC), or non-muscle invasive bladder cancer (NMIBC). In some aspects, the bladder cancer is a metastatic urothelial carcinoma (mUC). In some aspects, the cancer is a breast cancer. Further aspects of breast cancer include a hormone receptor-positive (HR+) breast cancer, e.g., an estrogen receptor-positive (ER+) breast cancer, a progesterone receptor-positive (PR+) breast cancer, or an ER+/PR+ breast cancer. Other aspects of breast cancer include a HER2-positive (HER2+) breast cancer. Yet other aspects of breast cancer include a triple-negative breast cancer (TNBC). In some aspects, the breast cancer is an early breast cancer. In some aspects, the cancer is a lung cancer. Further aspects of lung cancer include an epidermal growth factor receptor-positive (EGFR+) lung cancer. Other aspects of lung cancer include an epidermal growth factor receptor-negative (EGFR−) lung cancer. Yet other aspects of lung cancer include a non-small cell lung cancer, e.g., a squamous lung cancer or a non-squamous lung cancer. Other aspects of lung cancer include a small cell lung cancer. In some aspects, the cancer is a head and neck cancer. Further aspects of head and neck cancer include a squamous cell carcinoma of the head & neck (SCCHN). In some aspects, the cancer is a kidney cancer. Further aspects of kidney cancer include a renal cell carcinoma (RCC). In some aspects, the cancer is a liver cancer. Further aspects of liver cancer include a hepatocellular carcinoma. In some aspects, the cancer is a prostate cancer. Further aspects of prostate cancer include a castration-resistant prostate cancer (CRPC). In some aspects, the cancer is a metastatic form of a solid tumor. In some aspects, the metastatic form of a solid tumor is a metastatic form of a melanoma, a breast cancer, a colorectal cancer, a lung cancer, a head and neck cancer, a bladder cancer, a kidney cancer, an ovarian cancer, a pancreatic cancer, or a prostate cancer. In some aspects, the cancer is a metastatic urothelial carcinoma (mUC). In some aspects, the cancer is a non-solid tumor cancer. Non-solid tumor cancers include, but are not limited to, a B-cell lymphoma. Further aspects of B-cell lymphoma include, e.g., a chronic lymphocytic leukemia (CLL), a diffuse large B-cell lymphoma (DLBCL), a follicular lymphoma, myelodysplastic syndrome (MDS), a non-Hodgkin lymphoma (NHL), an acute lymphoblastic leukemia (ALL), a multiple myeloma, an acute myeloid leukemia (AML), or a mycosis fungoides (MF).

B. Combination Therapies

In some aspects of the above-described methods of treatment and prophylaxis, the method comprises administering to the individual at least one additional therapy (e.g., one, two, three, four, or more than four additional therapies). The modulator may be administered to the individual prior to, concurrently with, or after the at least one additional therapy.

C. Methods of Delivery

The compositions utilized in the methods described herein (e.g., a modulator of a protein-protein interaction described herein, e.g., a small molecule, an antibody, an antigen-binding fragment, a peptide, a mimic, an antisense oligonucleotide, or an siRNA) can be administered by any suitable method, including, for example, intravenously, intramuscularly, subcutaneously, intradermally, percutaneously, intraarterially, intraperitoneally, intralesionally, intracranially, intraarticularly, intraprostatically, intrapleurally, intratracheally, intrathecally, intranasally, intravaginally, intrarectally, topically, intratumorally, peritoneally, subconjunctivally, intravesicularly, mucosally, intrapericardially, intraumbilically, intraocularly, intraorbitally, orally, transdermally, intravitreally (e.g., by intravitreal injection), by eye drop, by inhalation, by injection, by implantation, by infusion, by continuous infusion, by localized perfusion bathing target cells directly, by catheter, by lavage, in cremes, or in lipid compositions. The compositions utilized in the methods described herein can also be administered systemically or locally. The method of administration can vary depending on various factors (e.g., the compound or composition being administered and the severity of the condition, disease, or disorder being treated). In some aspects, a modulator of a protein-protein interaction is administered intravenously, intramuscularly, subcutaneously, topically, orally, transdermally, intraperitoneally, intraorbitally, by implantation, by inhalation, intrathecally, intraventricularly, or intranasally. Dosing can be by any suitable route, e.g., by injections, such as intravenous or subcutaneous injections, depending in part on whether the administration is brief or chronic. Various dosing schedules including but not limited to single or multiple administrations over various time-points, bolus administration, and pulse infusion are contemplated herein.

A modulator of a protein-protein interaction described herein (and any additional therapeutic agent) may be formulated, dosed, and administered in a fashion consistent with good medical practice. Factors for consideration in this context include the particular disorder being treated, the particular mammal being treated, the clinical condition of the individual patient, the cause of the disorder, the site of delivery of the agent, the method of administration, the scheduling of administration, and other factors known to medical practitioners. The modulator need not be, but is optionally formulated with and/or administered concurrently with one or more agents currently used to prevent or treat the disorder in question. The effective amount of such other agents depends on the amount of the modulator present in the formulation, the type of disorder or treatment, and other factors discussed above. These are generally used in the same dosages and with administration routes as described herein, or about from 1 to 99% of the dosages described herein, or in any dosage and by any route that is empirically/clinically determined to be appropriate.

### VI. Methods of Identifying Biological Vesicles Having Altered Binding Profiles

In some aspects, the disclosure features a method for identifying a biological vesicle (BV) having an altered binding profile, the method comprising (a) providing a collection of target polypeptides that are immobilized on one or more solid surfaces; (b) contacting the collection of step (a) with a BV of interest; (c) detecting an interaction between the BV of interest and the at least one target polypeptide, thereby identifying an interaction profile; and (d) comparing the interaction profile of the BV of interest to the interaction profile of a control BV, wherein a difference between the interaction profile of the BV of interest and the interaction profile of the control BV identifies the BV of interest as one having an altered binding profile.

In some aspects, the collection of target polypeptides comprises the extracellular domains of at least 5%, at least 6%, at least 7%, at least 8%, at least 9%, at least 10%, at least 11%, at least 12%, at least 13%, at least 14%, at least 15%, at least 16%, at least 17%, at least 18%, at least 19%, at least 20%, at least 21%, at least 22%, at least 23%, at least 24%, at least 25%, at least 26%, at least 27%, at least 28%, at least 29%, at least 30%, at least 31%, at least 32%, at least 33%, at least 34%, at least 35%, at least 36%, at least 37%, at least 38%, at least 39%, at least 40%, at least 41%, at least 42%, at least 43%, at least 44%, at least 45%, at least 46%, at least 47%, at least 48%, at least 49%, at least 50%, at least 51%, at least 52%, at least 53%, at least 54%, at least 55%, at least 56%, at least 57%, at least 58%, at least 59%, at least 60%, at least 61%, at least 62%, at least 63%, at least 64%, at least 65%, at least 66%, at least 67%, at least 68%, at least 69%, at least 70%, at least 71%, at least 72%, at least 73%, at least 74%, at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% of the proteins of Table 4, e.g., 5%-15%, 15%-25%, 25%-35%, 35%-455, 45%-55%, 55%-65%, 65%-75%, 75%-85%, 85%-95%, or 95%-100% of the proteins of Table 4.

In some aspects, the collection of target polypeptides comprises the extracellular domains of at least 100, at least 150, at least 200, at least 250, at least 300, at least 350, at least 400, at least 450, at least 500, at least 550, at least 600, at least 650, at least 700, at least 750, at least 800, at least 850, at least 900, at least 950, at least 1000, at least 1050, at least 1100, at least 1150, or all 1195 of the proteins of Table 4, e.g., comprise the extracellular domains of 100-150, 150-200, 200-250, 250-300, 300-350, 350-400, 400-450, 450-500, 500-550, 550-600, 600-650, 650-700, 750-800, 800-850, 850-900, 900-950, 950-1000, 1000-1050, 1050-1100, 1100-1150, or all 1195 of the polypeptides of Table 4.

In some aspects, the collection of target polypeptides comprises the extracellular domains of at least 25% of the proteins of Table 4. In some aspects, the collection of target polypeptides comprises the extracellular domains of at least 50% of the proteins of Table 4. In some aspects, the collection of target polypeptides comprises the extracellular domains of at least 75% of the proteins of Table 4. In some aspects, the collection of target polypeptides comprises the extracellular domains of at least 90% of the proteins of Table 4. In some aspects, the collection of target polypeptides comprises the extracellular domains of all of the proteins of Table 4.

In some aspects, the extracellular domain of the prey protein (e.g., STM protein) has a native conformation, e.g., a conformation observed in the wild-type protein. In some aspects, the extracellular domain of the prey protein (e.g., STM protein) comprises a native post-translational modification.

In some aspects, the BV of interest is an engineered BV, e.g., a BV derived from a parent cell that has been modified (e.g., modified to express a heterologous protein, e.g., a receptor). The control BV may be, e.g., a BV produced by a control process or a BV derived from an unmodified parent cell.

In some aspects, the BV is a BV for use as a drug delivery vehicle. The control BV may be, e.g., a BV having a desired characteristic of a drug delivery vehicle. In some aspects, the method is used for quality control, e.g., to detect unexpected changes in the binding profile of EVs (e.g., changes arising from the overexpression of target receptors, addition of therapeutics, or modifications of the cells generating the EVs).

In some aspects, the BV of interest is derived from a sample from a subject. In some aspects, the method is used for unbiased profiling of the receptors for EVs from samples from a subject (e.g., used for comparison of EVs produced by different tissues, cell types, tumor cell lines, or immune cells. In some aspects, the BV of interest and the control BV are derived from different tissues or different cell types. In some aspects, the BV of interest is derived from a diseased tissue (e.g., tumor tissue), and the control BV is derived from healthy tissue.

### VII. Methods of Characterizing Interaction Profiles of Cell Lines

A. Methods of Characterizing Interaction Profiles of Cell Lines

In some aspects, the disclosure features a method for characterizing an interaction profile of a cell line, the method comprising (a) modifying the cell line to comprise a membrane-budding agent; and (b) characterizing an interaction profile of a biological vesicle (BV) produced by the cell line.

In some aspects, the disclosure features a method for characterizing an interaction profile of a cell line that has been modified to comprise a membrane-budding agent, the method comprising characterizing an interaction profile of a BV produced by the cell line.

In some aspects, the cell line may be a mammalian cell line. In some aspects, the mammalian cell line is a neuronal cell line, a fibroblast cell line, or an immune cell line. In some aspects, the immune cell line comprises one or more of T-cells, B-cells, or monocytes (e.g., consists of T-cell, or consists of B-cells, or consists of monocytes), e.g., is a T cell line, a B cell line, or a monocyte cell line.

In some aspects, the cell line is a mammalian cell line representing (e.g., derived from) a tissue type of interest, (e.g., a diseased or healthy tissue type) or a cell type of interest or a cell line representing (e.g., derived from) a tumor of interest (e.g., a tumor cell line). In some aspects, the cell line (e.g., mammalian cell line) is derived from a sample from a subject, e.g., a subject having a disease.

Exemplary membrane-budding agents are provided in Section III(C) herein. In some aspects, the membrane-budding agent is selected from the group consisting of a HIV gag protein, Acyl.Hrs, ARRDC1, and ARF6. In some aspects, the membrane-budding agent is a HIV gag protein. In some aspects, expression of the membrane-budding agent in the cell line is inducible. Exemplary constructs for inducible expression of the membrane-budding agent that may be introduced into the cell line include (a) an inducible promoter that relieves or suppresses the expression of the membrane-budding agent after the addition of a small molecule (e.g., a cell-permeable small molecule), e.g., the T-REX™ System; (b) a small molecule-induced degradation system in which the membrane-budding agent is rapidly degraded upon induction (e.g., the TIR1 auxin inducible degron (AID) system); and (c) a small molecule-induced stabilization system in which the membrane-budding agent comprises a degradation domain and the protein is protected from degradation upon induction (e.g., the Shield-1-FKBP system). Integration of constructs can be performed, e.g., by using CRISPR-Cas9 or genome engineering techniques to insert the construct into a safe-harbor locus. Alternatively, methods of random integration such as the PiggyBac Transposon System (SBI) may be used. Expression of the membrane-budding agent in the cell line may be induced at any desired time point; thus, production of BVs be the cell line may be induced at any time point.

In some aspects, characterizing the interaction profile of the BV comprises determining a level of one or more membrane-associated proteins of interest (e.g., one or more receptors of interest) on the BV.

In some aspects, characterizing the interaction profile of the BV is performed using a method comprising (a) providing a collection of target polypeptides that are immobilized on one or more solid surfaces; (b) contacting the collection of target polypeptides in step (a) with the BV; and (c) detecting an interaction between the BV and the at least one target polypeptide of the collection of target polypeptides, thereby identifying an interaction profile.

In some aspects, the collection of target polypeptides comprises the extracellular domains of at least 5%, at least 6%, at least 7%, at least 8%, at least 9%, at least 10%, at least 11%, at least 12%, at least 13%, at least 14%, at least 15%, at least 16%, at least 17%, at least 18%, at least 19%, at least 20%, at least 21%, at least 22%, at least 23%, at least 24%, at least 25%, at least 26%, at least 27%, at least 28%, at least 29%, at least 30%, at least 31%, at least 32%, at least 33%, at least 34%, at least 35%, at least 36%, at least 37%, at least 38%, at least 39%, at least 40%, at least 41%, at least 42%, at least 43%, at least 44%, at least 45%, at least 46%, at least 47%, at least 48%, at least 49%, at least 50%, at least 51%, at least 52%, at least 53%, at least 54%, at least 55%, at least 56%, at least 57%, at least 58%, at least 59%, at least 60%, at least 61%, at least 62%, at least 63%, at least 64%, at least 65%, at least 66%, at least 67%, at least 68%, at least 69%, at least 70%, at least 71%, at least 72%, at least 73%, at least 74%, at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% of the proteins of Table 4, e.g., 5%-15%, 15%-25%, 25%-35%, 35%-45%, 45%-55%, 55%-65%, 65%-75%, 75%-85%, 85%-95%, or 95%-100% of the proteins of Table 4.

In some aspects, the collection of target polypeptides comprises the extracellular domains of at least 100, at least 150, at least 200, at least 250, at least 300, at least 350, at least 400, at least 450, at least 500, at least 550, at least 600, at least 650, at least 700, at least 750, at least 800, at least 850, at least 900, at least 950, at least 1000, at least 1050, at least 1100, at least 1150, or all 1195 of the proteins of Table 4, e.g., comprise the extracellular domains of 100-150, 150-200, 200-250, 250-300, 300-350, 350-400, 400-450, 450-500, 500-550, 550-600, 600-650, 650-700, 750-800, 800-850, 850-900, 900-950, 950-1000, 1000-1050, 1050-1100, 1100-1150, or all 1195 of the polypeptides of Table 4.

In some aspects, the collection of target polypeptides comprises the extracellular domains of at least 25% of the proteins of Table 4. In some aspects, the collection of target polypeptides comprises the extracellular domains of at least 50% of the proteins of Table 4. In some aspects, the collection of target polypeptides comprises the extracellular domains of at least 75% of the proteins of Table 4. In some aspects, the collection of target polypeptides comprises the extracellular domains of at least 90% of the proteins of Table 4. In some aspects, the collection of target polypeptides comprises the extracellular domains of all of the proteins of Table 4.

In some aspects, the extracellular domain of the prey protein (e.g., STM protein) has a native conformation, e.g., a conformation observed in the wild-type protein. In some aspects, the extracellular domain of the prey protein (e.g., STM protein) comprises a native post-translational modification.

In some aspects, the method further comprises characterizing a cytoplasmic protein profile of the BV (e.g., comprises characterizing proteins present in the lumen of the BV).

B. Methods of Identifying Changes in Interaction Profiles of Cell Lines

In some aspects, the disclosure features a method for identifying a change in the interaction profile of a cell line, the method comprising (a) modifying the cell line to comprise a membrane-budding agent; (b) characterizing an interaction profile of a BV produced by the cell line at a first time point; (c) characterizing an interaction profile of a BV produced by the cell line at a second time point; and (d) comparing the interaction profile of the BV produced at the first time point to that of the BV produced at the second time point, wherein a difference between the interaction profile of the BV produced at the first time point and that of the BV produced at the second time point identifies a change in the interaction profile of the cell line.

In some aspects, the disclosure features a method for identifying a change in the interaction profile of a cell line that has been modified to comprise a membrane-budding agent, the method comprising (a) characterizing an interaction profile of a BV produced by the cell line at a first time point; (b) characterizing an interaction profile of a BV produced by the cell line at a second time point; and (c) comparing the interaction profile of the BV produced at the first time point to that of the BV produced at the second time point, wherein a difference between the interaction profile of the BV produced at the first time point and that of the BV produced at the second time point identifies a change in the interaction profile of the cell line.

In some aspects, the method comprises exposing the cell line to a stimulus following the first time point and before the second time point; thus, the method can be used to identify changes in the interaction profile of the cell line that occur as a result of exposure to the stimulus. The stimulus may be, e.g., a condition or agent that induces signaling, a condition or agent that induces a disease-related state, and/or a condition or agent that induces differentiation. In some aspects, the cell line is an immune cell line and the disease-related state is immune exhaustion.

In other aspects, the method does not comprise exposing the cell line to a stimulus. For example, in some aspects, the first time point and the second time point are selected to be at different stages during differentiation of a cell line, or BVs produced at a first time point and a second time point are assessed to determine whether the cell line has differentiated between the time points.

In some aspects, the method further comprises characterizing an interaction profile of a BV produced by the cell line at one or more additional time points, e.g., at 1, 2, 3, 4, 5, 6, 7, 8, 9, 10, or more than 10 additional time points. In some aspects, the method comprises exposing the cell line to a stimulus following the second time point, e.g., exposing the cell line to a stimulus before the one or more additional time points.

The cell line may be a mammalian cell line. In some aspects, the mammalian cell line is a neuronal cell line, a fibroblast cell line, or an immune cell line. In some aspects, the immune cell line comprises one or more of T-cells, B-cells, or monocytes (e.g., consists of T-cell, or consists of B-cells, or consists of monocytes), e.g., is a T cell line, a B cell line, or a monocyte cell line.

In some aspects, the cell line is a mammalian cell line representing (e.g., derived from) a tissue type of interest, (e.g., a diseased or healthy tissue type) or a cell type of interest or a cell line representing (e.g., derived from) a tumor of interest (e.g., a tumor cell line). In some aspects, the cell line (e.g., mammalian cell line) is derived from a sample from a subject, e.g., a subject having a disease.

Exemplary membrane-budding agents are provided in Section III(C) herein. In some aspects, the membrane-budding agent is selected from the group consisting of a HIV gag protein, Acyl.Hrs, ARRDC1, and ARF6. In some aspects, the membrane-budding agent is a HIV gag protein. In some aspects, expression of the membrane-budding agent in the cell line is inducible. Exemplary membrane-budding agents are provided in Section III(C) herein, and exemplary inducible constructs are described in Section VII(A), above. Expression of the membrane-budding agent in the parent cell line, and thus production of BVs by the parent cell line, may be induced at any desired time point. For example, in some aspects, expression of the membrane-budding agent is induced at the first time point, the second time point, and optionally at one or more additional time points. In some aspects, expression of the membrane-budding agent is not induced in the interval between the first time point and the second time point, e.g., the membrane-budding agent is not expressed in the interval between the first time point and the second time point.

In some aspects, characterizing the interaction profile of the BV produced by the cell line at a first time point and the BV produced by the cell line at a second time point comprises determining a level of one or more membrane-associated proteins of interest (e.g., one or more receptors of interest) on each of the BVs, as described in Section VII(A), above.

In some aspects, the method further comprises characterizing a cytoplasmic protein profile of the BV produced by the cell line at a first time point and the BV produced by the cell line at the second time point (e.g., comprises characterizing proteins present in the lumen of the BV produced by the cell line at the first time point and the BV produced by the cell line at the second time point).

C. Methods of Comparing Interaction Profiles of Cell Lines

In some aspects, the disclosure features a method for identifying a difference in the interaction profiles of two cell lines, the method comprising (a) modifying each of the cell lines to comprise a membrane-budding agent; (b) characterizing an interaction profile of a BV produced by the first cell line; (c) characterizing an interaction profile of a BV produced by the second cell line; and (d) comparing the interaction profile of the BV produced at the first cell line to that of the BV produced by the second cell line, wherein a difference between the interaction profile of the BV produced by the first cell line and that of the BV produced by the second cell line identifies a difference in the surface protein profiles of two cell lines.

In some aspects, the disclosure features a method for identifying a difference in the interaction profiles of two cell lines that have been modified to comprise a membrane-budding agent, the method comprising (a) characterizing an interaction profile of a BV produced by the first cell line; (b) characterizing an interaction profile of a BV produced by the second cell line; and (c) comparing the interaction profile of the BV produced at the first cell line to that of the BV produced by the second cell line, wherein a difference between the interaction profile of the BV produced by the first cell line and that of the BV produced by the second cell line identifies a difference in the surface protein profiles of two cell lines.

In some aspects, the first cell line and the second cell line are mammalian cell lines. In some aspects, the BV produced by the first cell line and the BV produced by the second cell line are derived from different tissues or different cell types. In some aspects, the BV produced by the first cell line is derived from a diseased tissue (e.g., tumor tissue), and the BV produced by the second cell line is derived from healthy tissue.

Exemplary membrane-budding agents are provided in Section III(C) herein. In some aspects, the membrane-budding agent is selected from the group consisting of a HIV gag protein, Acyl.Hrs, ARRDC1, and ARF6. In some aspects, the membrane-budding agent is a HIV gag protein. In some aspects, expression of the membrane-budding agent in the cell line is inducible. Exemplary membrane-budding agents are provided in Section III(C) herein, and exemplary inducible constructs are described in Section VII(A), above.

In some aspects, characterizing the interaction profile of the BV produced by the cell line at a first time point and the BV produced by the cell line at a second time point comprises determining a level of one or more membrane-associated proteins of interest (e.g., one or more receptors of interest) on each of the BVs, as described in Section VII(A), above.

In some aspects, the method further comprises characterizing a cytoplasmic protein profile of the BV produced by the cell line at a first time point and the BV produced by the cell line at the second time point.

In another aspect, the disclosure features a BV comprising a heterologous membrane-budding agent, wherein the BV is produced by a process comprising (i) providing a parent cell line that has been modified to express the membrane-budding agent under inducible control; (ii) inducing expression of the membrane-budding agent, and (iii) isolating the BV from the parent cell line. In some aspects, the membrane-budding agent is selected from the group consisting of a HIV gag protein, Acyl.Hrs, ARRDC1, and ARF6. In some aspects, the membrane-budding agent is a HIV gag protein. In some aspects, the parent cell line is a mammalian cell line. In some aspects, the BV is an extracellular vesicle (EV).

### VIII. Methods of Assessing Membrane Protein Activity Using Biological Vesicles

In some aspects, the disclosure features a method for assessing an enzymatic activity of a membrane-associated protein, the method comprising conducting an assay for enzymatic activity on a biological vesicle (BV) comprising the protein.

Exemplary BVs are described in Section III(B) herein. In some aspects, the membrane-associated protein is endogenous to the BV and/or the parent cell thereof, i.e., the enzymatic activity of an endogenous membrane-associated protein is assessed using the method.

In other aspects, the BV membrane-associated protein is a heterologous membrane-associated protein present on the outer face of the BV, i.e., the enzymatic activity of the heterologous membrane-associated protein is assessed using the method. BVs comprising heterologous membrane-associated proteins are described in Section II herein.

In some aspects, the heterologous membrane-associated protein is a full-length protein. In other aspects, the heterologous membrane-associated protein is a protein fragment. In some aspects, the heterologous membrane-associated protein comprises a protein fragment, a tag, and an anchor (e.g., an anchor that tethers the protein fragment to the surface of a membrane of the BV, e.g., a glycosylphosphatidyl-inositol (GPI) polypeptide).

In some aspects, the membrane-associated protein is a peptidase and the assay for enzymatic activity is an assay for peptidase activity, e.g., an assay for degradation of one or more known or putative substrates of the peptidase.

In some aspects, the membrane-associated protein is a protease and the assay for enzymatic activity is an assay for protease activity, e.g., an assay for degradation of one or more known or putative substrates of the protease.

In some aspects, the membrane-associated protein is a kinase and the assay for enzymatic activity is an assay for kinase activity, e.g., an assay for phosphorylation of one or more known or putative substrates of the kinase.

In some aspects, the membrane-associated protein is a phosphatase and the assay for enzymatic activity is an assay for phosphatase activity, e.g., an assay for dephosphorylation of one or more known or putative substrates of the phosphatase.

### IX. Methods of Purifying Biological Vesicles

In some aspects, the disclosure features a method of purifying a biological vesicle (BV) from a culture medium (e.g., a liquid culture medium) or a sample from a subject (e.g., a liquid sample, e.g., a urine sample, a blood sample, or a digested tissue sample), the method comprising contacting a BV (e.g., a BV in a culture medium) with a solid surface comprising one or more of the generic vesicle binder proteins of Table 8 or Table 9.

TABLE 8
Generic vesicle binder genes

AGER
ALK
AMICA1
APLP2
APP
ASGR1
BTC
C14orf180
C19orf59
CD177
CD22
CD300A
CD300E
CD300LB
CD300LF
CD300LG
CD33
CD6
CD72
CILP
CLEC10A
CLEC14A
CLEC4A
CLEC4G
CLSTN2
CLSTN3
CR2
CSF1R
DDOST
DPCR1
EDA
EPHA6
EPHA7
EPHB1
EPHB2
EPHB3
FCAMR
FCRL2
FGFR4
FGFRL1
FIBCD1
FLT1
FSTL5
GFRA1
GFRA2
GLG1
HAVCR1
HAVCR2
HBEGF
ICAM5
IFNLR1
IGFBPL1
IGSF3
IL15RA
IL1RL1
KLRK1
KREMEN1
KREMEN2
LAG3
LDLR
LILRA1
LILRA3
LILRA6
LILRB2
LILRB5
LRPAP1
MAG
MRC1
MSR1
NFASC
NLGN3
NRG2
NRP1
NRP2
OLR1
PLXDC2
PRND
PRNP
PSG4
PSG5
PTPRB
PTPRD
PTPRF
PTPRS
PXDN
RARRES1
RNF13
ROBO1
ROBO2
RTN4R
SARAF
SELL
SELP
SIGLEC10
SIGLEC14
SIGLEC15
SIGLEC5
SIGLEC6
SIGLEC7
SIGLEC8
SIGLEC9
SIRPA
SIRPG
SPRN
TIE1
TIMD4
TMEM132A
TNFRSF11B
TREML2
WBP1

TABLE 9
List of Generic Vesicle Binder Genes and Scores

| Gene Name | Average Score |
|---|---|
| HAVCR1 | 1.463141 |
| MAG | 1.120588 |
| TIMD4 | 0.984632 |
| SIGLEC7 | 0.972398 |
| CD300LF | 0.805923 |
| SIRPA | 0.76706 |
| SIGLEC9 | 0.737391 |
| MRC1 | 0.688295 |
| SIGLEC8 | 0.674243 |
| CD300LG | 0.669373 |
| MSR1 | 0.572835 |
| SIGLEC10 | 0.558422 |
| CD22 | 0.544031 |
| IGSF3 | 0.533152 |
| SIRPG | 0.495548 |
| FCRL2 | 0.486656 |
| ROBO1 | 0.463355 |
| SELP | 0.458027 |
| CLEC10A | 0.426572 |
| C19orf59 | 0.419371 |
| SIGLEC15 | 0.414953 |
| TMEM132A | 0.413924 |
| FGFRL1 | 0.401978 |
| ASGR1 | 0.401957 |
| GFRA1 | 0.394782 |
| CLEC14A | 0.383711 |
| TREML2 | 0.368332 |
| FLT1 | 0.364596 |
| PTPRD | 0.360015 |
| PTPRB | 0.353434 |
| PRND | 0.344341 |
| GFRA2 | 0.339367 |
| NRP1 | 0.331184 |
| RNF13 | 0.32993 |
| APLP2 | 0.320281 |
| SARAF | 0.318255 |
| HBEGF | 0.309675 |
| EPHB2 | 0.299617 |
| FSTL5 | 0.296042 |
| CD300A | 0.294351 |
| NRP2 | 0.283246 |
| LDLR | 0.280714 |
| PILRA | 0.266112 |
| IFNLR1 | 0.265823 |
| SIGLEC5 | 0.264558 |
| NRG2 | 0.256761 |
| LILRA3 | 0.251963 |
| EPHB1 | 0.248776 |
| OLR1 | 0.246329 |
| CD33 | 0.240488 |
| CD177 | 0.240066 |
| CD72 | 0.23771 |
| LILRB2 | 0.233592 |
| PRNP | 0.232195 |
| RARRES1 | 0.231731 |
| EPHA6 | 0.228359 |
| FIBCD1 | 0.225934 |
| AGER | 0.225417 |
| PTPRS | 0.221677 |
| ICAM5 | 0.22125 |
| C14orf180 | 0.220482 |
| NLGN3 | 0.21094 |
| BTC | 0.202919 |
| PLXDC2 | 0.199299 |
| EPHB3 | 0.197941 |
| IL15RA | 0.193258 |
| LAG3 | 0.193037 |
| LILRA1 | 0.190473 |
| MEGF10 | 0.189179 |
| ROBO2 | 0.181367 |
| IL1RL1 | 0.177439 |
| PSG4 | 0.169163 |
| LILRB5 | 0.162532 |
| SIGLEC6 | 0.156384 |
| LILRA6 | 0.153831 |
| ALK | 0.15204 |
| PSG5 | 0.147583 |
| CD2 | 0.137838 |
| PTPRF | 0.118992 |
| SORT1 | 0.110567 |

Exemplary BVs are described in Section III(B) herein. The BV may be an unmodified BV or may comprise an exogenous protein (e.g., a heterologous membrane-associated protein, e.g., a full-length heterologous membrane-associated protein or a heterologous membrane-associated protein comprising a protein fragment, a tag, and an anchor) as described in Section II herein. In some aspects, the BV comprises a membrane-budding agent and is produced by a process comprising (i) providing a parent cell that has been modified to express the membrane-budding agent and (ii) isolating the BV from the parent cell.

The solid surface may be any stationary surface suitable for affinity purification, e.g., a column (e.g., a column comprising Protein A-functionalized beads), a bead, a plane, or a plate.

The solid surface may be modified to comprise the one or more of the proteins of Table 8 or Table 9 using any appropriate method. In some aspects, the solid surface comprises a moiety with affinity for the one or more proteins of Table 8 or Table 9, and the solid surface is contacted with (e.g., washed with) the one or more proteins of Table 8 or Table 9, thus immobilizing the one or more proteins of Table 8 or Table 9 on the solid surface. The one or more proteins of Table 8 or Table 9 may be modified to comprise a moiety (e.g., a tag), and the affinity of the moiety comprised by the solid surface may be to the moiety or tag. For example, in one aspect, the solid surface comprises Protein A and the one or more proteins of Table 8 or Table 9 have been modified to comprise an Fc region.

In some aspects, the solid surface comprises a single protein of Table 8 or Table 9. In some aspects, the solid surface comprises HAVCR1, MAG, TIMD4, SIGLEC7, CD300LF, SIRPA, SIGLEC9, MRC1, SIGLEC8, or CD300LG. In some aspects, the solid surface comprises HAVCR1. In some aspects, the solid surface comprises MAG. In some aspects, the solid surface comprises SIGLEC7. In some aspects, the solid surface comprises CD300LF. In some aspects, the solid surface comprises 2, 3, 4, 5, 6, 7, 8, 9, or all 10 of HAVCR1, MAG, TIMD4, SIGLEC7, CD300LF, SIRPA, SIGLEC9, MRC1, SIGLEC8, and CD300LG.

In some aspects, the solid surface comprises 2, 3, 4, 5, 6, 7, 8, 9, 10, 11, 12, 13, 14, 15, 16, 17, 18, 19, 20, 21, 22, 23, 24, 25, or more than 25 proteins of Table 8 or Table 9, e.g., comprises 2-5, 5-10, 10-15, 15-20, or 20-25 of the proteins of Table 8 or Table 9. In some aspects, the solid surface comprises two of the proteins of Table 8 or Table 9. In some aspects, the solid surface comprises three of the proteins of Table 8 or Table 9. In some aspects, the solid surface comprises four of the proteins of Table 8 or Table 9. In some aspects, the solid surface comprises five of the proteins of Table 8 or Table 9.

In some aspects, the one or more of the proteins of Table 8 or Table 9 are human proteins. In some aspects, the BVs are derived from human cells.

In some aspects, contacting the BV with the solid surface comprises flowing culture medium or a sample from a subject (e.g., a liquid sample, e.g., a urine sample, a blood sample, or a digested tissue sample) comprising the BV over the solid surface. In some aspects, the culture medium is conditioned medium. In some aspects, the culture medium or sample comprises one or more parent cells from which the BV was produced. In other aspects, the culture medium or sample does not comprise parent cells, e.g., has been processed to remove parent cells.

In some aspects, the method further comprises detaching (e.g., eluting) the BV from the solid surface. BVs may be eluted using any method suitable to detach the BV from the solid surface. For example, washes (e.g., harsh washes, e.g., high-salt washes) and/or appropriate ligands may be used to detach EVs. For example, in some aspects, the one or more proteins of Table 8 or Table 9 comprise a member of the SIGLEC family of proteins and one or more sialoglycans is used in elution.

In some aspects, the solid surface is a column comprising Protein A-functionalized beads and the method comprises flowing the conditioned media comprising the one or more of the proteins of Table 8 or Table 9 over the column, wherein the one or more proteins of Table 8 or Table 9 have been modified to comprise an Fc region (e.g., a human Fc region), thus immobilizing the one or more proteins of Table 8 or Table 9; flowing the culture medium or sample from the subject comprising the BVs over the column; and eluting the BVs from the column.

In some aspects, the method comprises an ultracentrifugation-based cleanup step. In other aspects, the method does not comprise ultracentrifugation.

In some aspects, the method is used for large-scale purification of BVs. In some aspects, the method is performed using a sample volume of at least 10 mL, i.e., at least 10 mL of a culture medium or a sample from a subject is processed according to the method. For example, in some aspects, the method is performed using a sample volume of at least 15 mL, 20 mL, 25 mL, 30 mL, 35 mL, 40 mL, 45 mL, 50 mL, 55 mL, 60 mL, 65 mL, 70 mL, 75 mL, 80 mL, 85 mL, 90 mL, 95 mL, or 100 mL (e.g., is performed using a sample volume of 10-20 mL, 20-30 mL, 30-40 mL, 40-50 mL, 50-60 mL, 60-70 mL, 70-80 mL, 80-90 mL, or 90-100 mL). In some aspects, the method is performed using a sample volume of at least 50 mL. In some aspects, the method is performed using a sample volume of at least 100 mL. For example, in some aspects, the method is performed using a sample volume of at least 150 mL, 200 mL, 250 mL, 300 mL, 350 mL, 400 mL, 450 mL, 500 mL, 550 mL, 600 mL, 650 mL, 700 mL, 750 mL, 800 mL, 850 mL, 900 mL, 950 mL, or 1 L (e.g., is performed using a sample volume of 100-200 mL, 200-300 mL, 300-400 mL, 400-500 mL, 500-600 mL, 600-700 mL, 700-800 mL, 800-900 mL, or 900 mL-1 L).

BVs purified according to these methods may be used in any of the methods described herein.

All patent, patent publication and literature references cited in the present specification are hereby incorporated by reference in their entirety.

### X. Examples

### Example 1. An Extracellular Vesicle-Based Screen for Detection of Extracellular Protein-Protein Interactions in Membranes

A. Background

Membrane proteins play an essential role in translating extracellular cues into intracellular responses. Their cell-surface exposure, resulting in increased accessibility to therapeutic molecules, and their ability to orchestrate cellular behavior makes them attractive drug targets. Therefore, it is unsurprising that, while they make up only ˜30% of human genes, they account for over 60% of all drug targets (Santos et al., Nat. Rev. Drug Discov., 16: 9-34, 2016.

The identification of receptor-ligand interactions is thus instrumental to understanding cellular communication occurring in the extracellular milieu. However, progress on the characterization of membrane proteins and their interaction partners has lagged far behind that of cytoplasmic proteins. This is in part due to difficulties in expressing membrane proteins in their native conformations and the lack of techniques that have sufficient sensitivity to detect the weak interactions common for membrane proteins (Martinez-Martin, J. Immunol. Res, 2017: 2197615, 2017; Wright, Mol. Biosyst. 5: 1405-1412, 2009). Methods designed for general protein-protein interaction discovery require either well-folded purified protein (e.g., microarray- or plate-based screening methods (Martinez-Martin, J. Immunol. Res, 2017: 2197615, 2017; Wright et al., Biochem. Soc. Trans, 38: 919-922, 2010)), or strong interactions that can survive extraction of proteins from membranes and washes (e.g. affinity purification-mass spectrometry (AP/MS) (Huttlin et al., bioRxiv, doi:10.1101/2020.01.19.905109, 2020)). While some newer approaches have been able to capture many membrane protein interactions by using multimerization to strengthen the weak interactions (Bushell et al., Genome Res., 18: 622-630, 2008; Husain et al., Mol. Cell. Proteomics, 18: 2310-2323, 2019), they may still miss key interactions because the proteins are removed from their native membrane contexts.

Physiological membranes contain a complex mix of lipids, sterols, proteins and glycans that can participate in interactions (Goñi, Biochim. Biophys. Acta—Biomembr., 1838: 1467-1476, 2014). In addition, membranes can strengthen individually weak protein-protein interactions by clustering and orienting (Banjade et al., Elife, 3: 1-24, 2014; Taylor et al., Cell, 169: 108-119e.20, 2017; Hu et al., Proc. Nat. Acad. Sci. U.S.A., 110: 15283-15288, 2013). These membrane-dependent aspects of the receptor-ligand interactions remain a major bottleneck in the development of screening methods. Recent advances in proximity-based techniques allow detection of interactions in membranes and have been instrumental for the study of transient binders (Geri et al., Science, 367: 1091-1097, 2020; Li et al., Cell, 180: 373-386.e15, 2020, Gingras et al., Curr. Opin. Chem. Biol., 48: 55-54, 2019). However, these techniques often struggle to distinguish direct interaction partners from nearby bystanders, typically focus on binding partners within the same cell (in-cis interactions), and are often incompatible with high-throughput studies. Alternative approaches such as nanodiscs and liposome particles enable membrane protein reconstitution and have been successfully employed to study challenging receptors (Rouck et al., FEBS Lett., 591: 2057-2088, 2017; De Franceschi et al., J. Cell Sci., 132: 2019). However, these methods require protein purification, which can disrupt native folds and rarely account for potential protein or non-protein cofactors. As a result, the extracellular protein cross-talk remains remarkably underrepresented in existing datasets (Wright et al., Biochem. Soc. Trans., 38: 919-922, 2010; Bausch-Fluck et al., Proc. Natl. Acad. Sci., 2018). These limitations underscore the need for additional techniques specifically designed for the study of membrane proteins, with sufficient throughput and sensitivity for the characterization of receptor interactomes.

The need for an in-membrane receptor display method has spurred several solutions that take advantage of machinery from enveloped viruses to incorporate receptors into mammalian membranes. For example, microarrays consisting of herpes simplex virions (VirD) displaying different membrane proteins have been used successfully as a GPCR library for ligand discovery (Hu et al., Proc. Natl. Acad. Sci. U.S.A., 110: 15283-15288, 2013; Da Syu et al., Nat. Commun., 10: 1-12, 2019. Additionally, extracellular vesicles containing the HIV gag protein, termed recombinant extracellular vesicles (rEVs), have been used to display multi-transmembrane proteins for immunization and antibody generation (Tucker et al., Proc. Natl. Acad. Sci. U.S.A., 115: E4990-E4999, 2018) as well as antibody selection and ligand-binding characterization (Willis et al., Biochemistry, 47: 6988-6990, 2008). These rEVs have lipid and protein compositions similar to the naturally occurring EVs that play a role in cellular communication and pathogenesis of multiple disease (Lavado-Garcia et al., J. Proteome Res., 19: 4516-4532, 2020; Geeurickx et al., Nat. Commun., 10: 1-12, 2019). Therefore, defining interactomes for rEVs can provide insights into both the binding profile of a displayed receptor-of-interest and basic EV biology.

To address the limitations of these previous approaches, an assay combining the robustness of a direct protein-protein interaction screen with the presentation of a target-of-interest in the context of a membrane was designed using EVs, e.g., rEVs as described above. EVs are nanometer-sized, lipid bilayer-delimited particles that are naturally secreted from cells (Colombo et al., Annu. Rev. Cell Dev. Biol., 30: 255-289, 2014). EVs contain proteins folded and inserted into their native membranes using the cell's endogenous machinery. Herein, EVs are shown to provide a protein-purification free method for obtaining binding-competent receptors. To take advantage of these naturally secreted particles, RDIMIS (Receptor-Display In Membranes Interaction Screen) (also called EVEXIS (extracellular vesicle (EV)-based extracellular interaction screen)), a new platform for receptor-ligand discovery, was developed. RDIMIS allows for rapid, unbiased identification of single-pass transmembrane (STM) protein interactomes by screening any protein-of-interest expressed on EVs against a comprehensive conditioned media library of STM ectodomains (Czajkowsky et al., EMBO Mol. Med., 4:1015-1028, 2012; Martinez-Martin et al., Cell, 174: 1158-1171.e19, 2018).

RDIMIS is a time-efficient method for the elucidation of receptor-ligand interactions that is receptor-agnostic and thus applicable to most targets of interest that can be incorporated in EVs. Profiling the interactome of a membrane protein requires only a plasmid for its expression. The EV context obviates time-consuming and uncertain protein purifications while retaining the advantages of studying proteins in a simplified, plate-based system. EVs use the endogenous cellular machinery to translate, fold and insert the protein into the membrane; therefore, the likelihood of expressing functional protein is optimal. Furthermore, capturing the STM library proteins directly from conditioned media eliminates all protein purification steps, enabling higher throughput studies, and again minimizing resource-consuming purification steps without compromising protein activity (Husain et al., Mol. Cell. Proteomics, 18: 2310-2323, 2019). This allows characterization of the interactome of a membrane protein-of-interest to be performed in about a week, limited primarily by the time it takes cells to produce proteins and EVs.

While the rEV-based approach enables the use of full-length untagged receptor molecules, use of gD-GPI tagged ectodomains can achieve similar results and skirts the need for elusive high-affinity antibodies to measure receptor incorporation. The gD-GPI tag works across receptor families and allows direct quantitative comparison of expression by BLI. This tagging strategy also enables the direct comparison of the interaction profiles of the ectodomain and full-length protein, quickly identifying interactions in which the transmembrane or cytoplasmic domains may play a role. Lastly, the gD-GPI tag can anchor non-membrane proteins, providing a way to study extracellular matrix or secreted factors in proximity to membranes.

rEVs also contain a cytoplasmic luminal space with cytoskeletal elements that can play roles in higher-order complex formation (Banjade et al., Elife, 3: 1-24, 2014; Keerthikumar et al., J. Mol. Biol., 428: 688-692, 2016). In addition to capturing the complexity of cellular membranes, the small size and stability of rEVs (Colombo and Raposo, Annu. Rev. Cell Dev. Biol., 30: 255-289, 2014) make them a very attractive vehicle for high-throughput screening in a physiologically relevant context. As such, the approach implemented in this study can be coupled to any library of choice, from high-coverage collections to more focused libraries such as receptor families or protein disease variants, enabling sensitive and relatively low resource-intensive identification of interactomes while maximizing query protein quality.

Beyond identification of protein interactomes and deorphanization of complex or hard-to-purify targets, the RDIMIS approach has wide applications in the study of receptor, membrane as well as human EV biology. RDIMIS identified over a hundred vesicle-specific binders for a population of HEK cell-derived, human EVs and showed that the interactome of recombinant and endogenously generated EVs were remarkably similar. This is consistent with previous works showing strong similarities between the proteomic and lipid compositions of rEVs and endogenous EVs (Lavado-Garcia et al., J. Proteome Res., 19: 4516-4532, 2020; Geeurickx et al., Nat. Commun., 10: 1-12, 2019). Therefore, these binders may shed light on endogenous EV tropism and signaling. Results indicate that EVs interact with major signaling proteins such as immunomodulatory proteins (i.e. SIGLEC family, LILR family and CD300 family), growth regulators proteins (i.e. growth factor receptors like FGFR4, FLT1 and NRP proteins and several receptor-tyrosine kinases) or neuronal proteins (i.e. APP and CLSTN proteins), supporting the idea that EVs mediate intercellular communication. This work sets the stage for future studies focused on disease-, tissue- or cell-specific EVs and their roles in cellular communication or immune responses. Given the throughput and reproducibility, this method provides a way to assess the quality of EVs on the global scale. As EVs are being explored as avenues for drug delivery (Vader et al., Adv. Drug Deliv. Rev, 106: 148-156, 2016), RDIMIS can provide a way to address concerns about variability, immunogenicity and off-target effects associated with EV complexity. Since this platform provides a unique tool to elucidate the players that influence EV functions at the molecular level, it can detect unexpected changes in the binding profile of EVs arising from the overexpression of target receptors, addition of therapeutics or modifications of the cells generating the EVs.

B. Extracellular Vesicles for Membrane Display

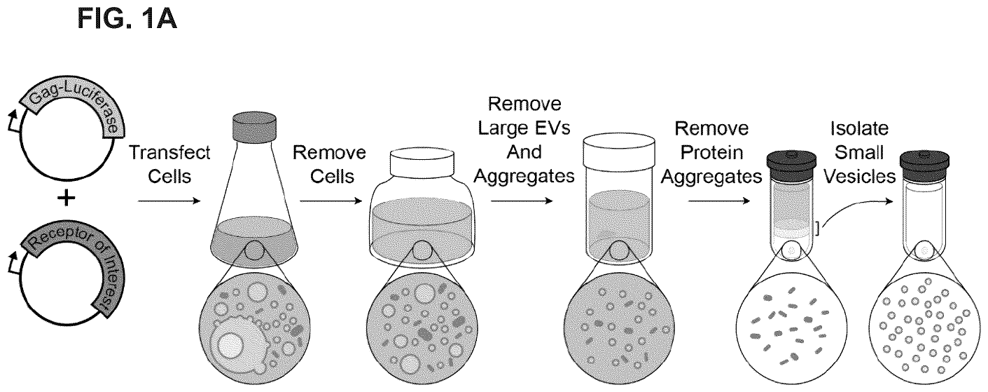

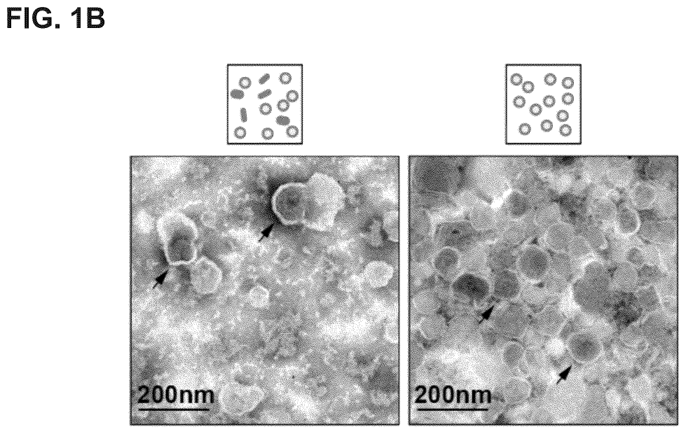

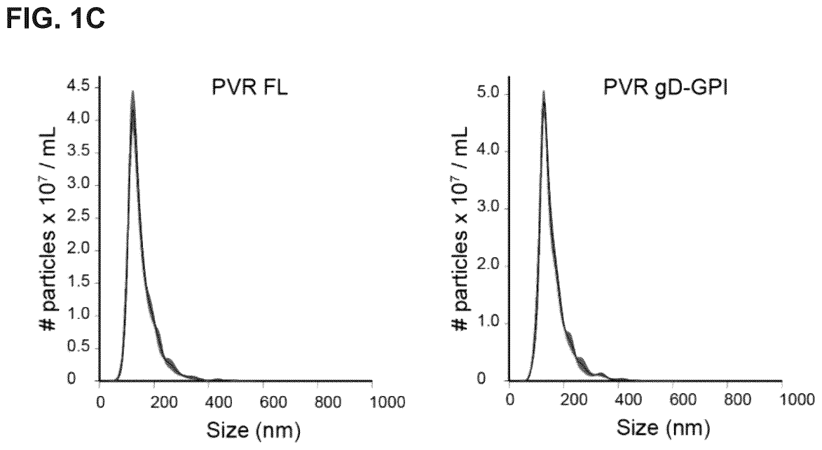

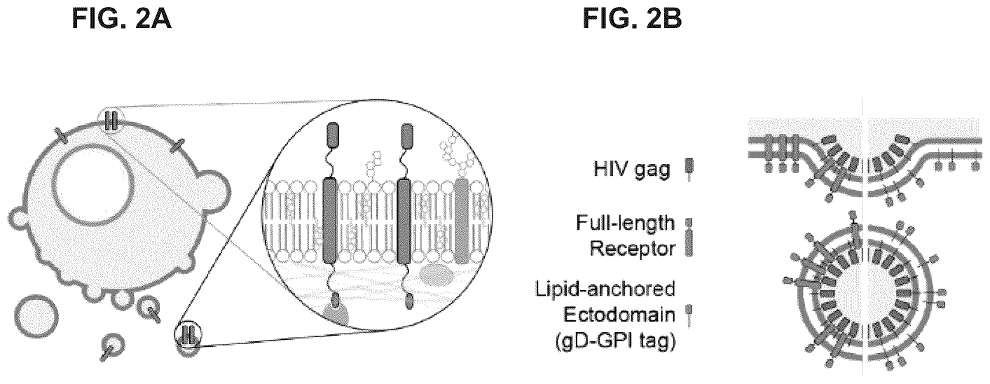

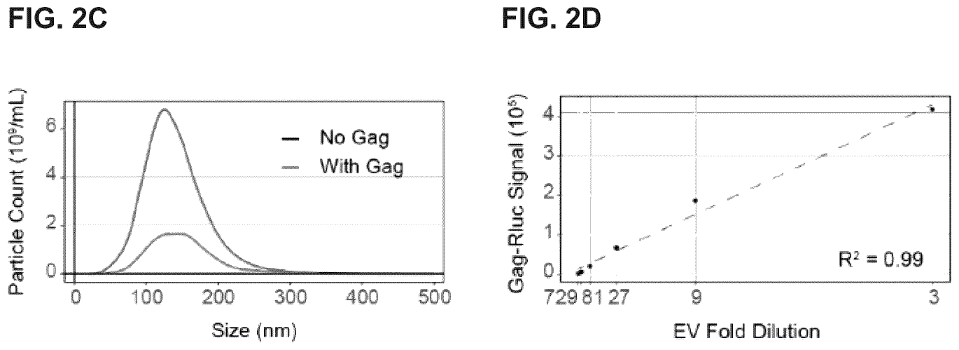

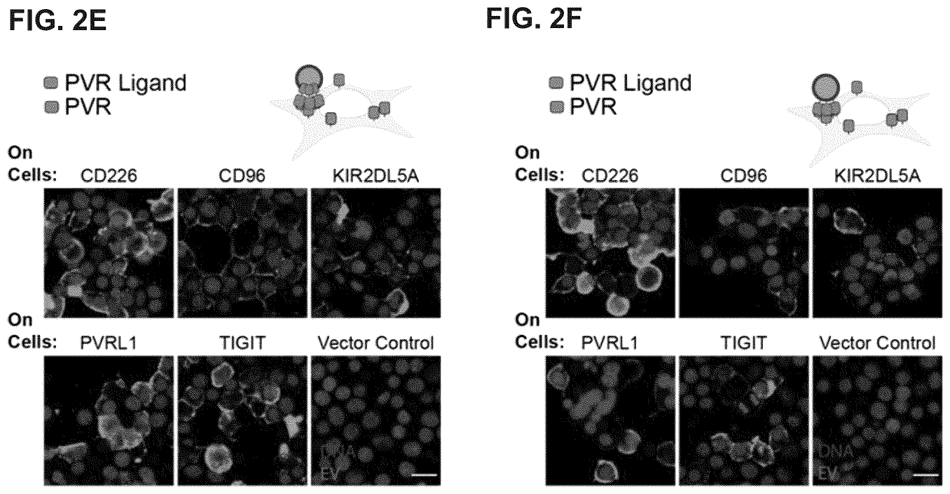

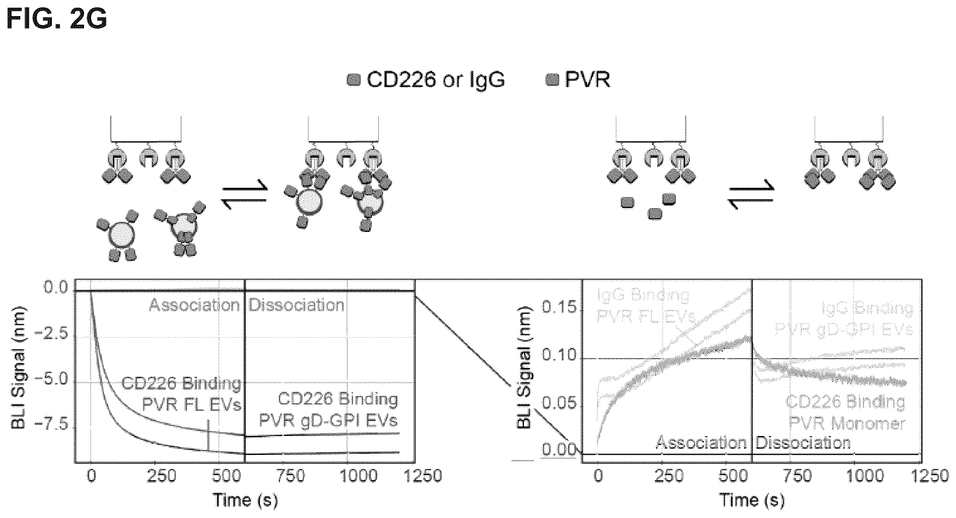

A method for receptor-ligand discovery in native membranes requires several parts. First, it must capture stable pieces of cellular membranes. This can be achieved by purifying EVs, lipid bilayer-contained particles naturally generated by most cells (FIGS. 1A-1C) (Colombo et al., Annu. Rev. Cell Dev. Biol., 30: 255-289, 2014). EVs incorporate a host of membrane-associated macromolecules (Keerthikumar et al., J. Mol. Biol., 428: 688-692, 2016), making them a microcosm of the cellular membrane environment (FIG. 2A). This environment often participates in membrane protein interactions, making EVs a very attractive basis for a high-throughput platform.

Second, large quantities of EVs are likely necessary for high-throughput, sensitive screening. In order to maximize EV production, EXPI293F™ cells, which provide an efficient system for protein expression, were chosen (Heath et al., Sci. Rep., 8: 1-12, 2008; Arena et al., MAbs, 11: 977-986, 2019). EXP1293F™ cells were cultured in EXP1293™ Expression Media, shaking, at 150 rpm. All cells were cultured at 37° C., 5% CO₂.

To further boost EV production (Geeurickx et al., Nat. Commun., 10: 1-12, 2019; Cervera et al., J. Biotechnol., 166: 152-165, 2013), cells were transfected with an HIV gag construct (FIG. 2B). Gag expression increased the yield of 20-500 nm vesicles by almost 4-fold (FIG. 2C).

Third, robust and quantitative methods are needed for detecting EVs. Cells release their EVs into a surrounding medium that also contains a complex and variable background of other molecules. These make it challenging to get reproducible quantitative data from downstream assays. To address this issue, a one-step density-based EV purification was optimized. This gave a pure and consistent population of rEVs free of aggregates (FIGS. 1A and 1B). Lastly, high-throughput screening necessarily shrinks the scale of each reaction. This creates a challenge for robust and quantitative detection of bound EVs. Renilla luciferase (Rluc) was therefore fused to the HIV gag construct. This gave robust luminescence signals linearly proportional to vesicle concentration over nearly three orders of magnitude (FIG. 2D).

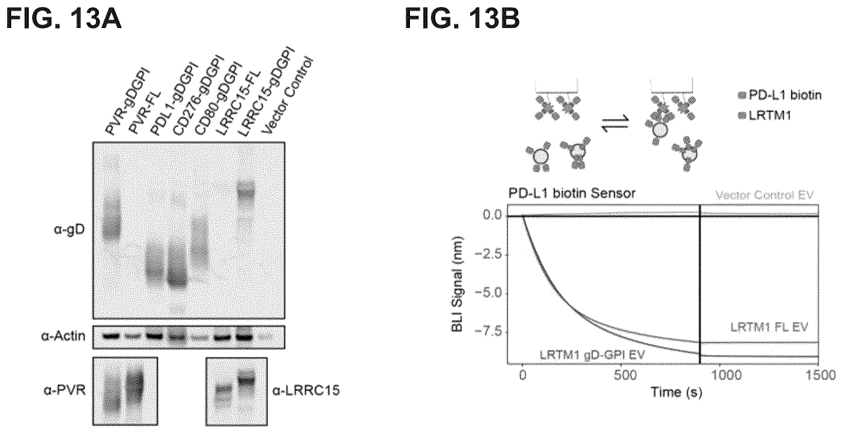

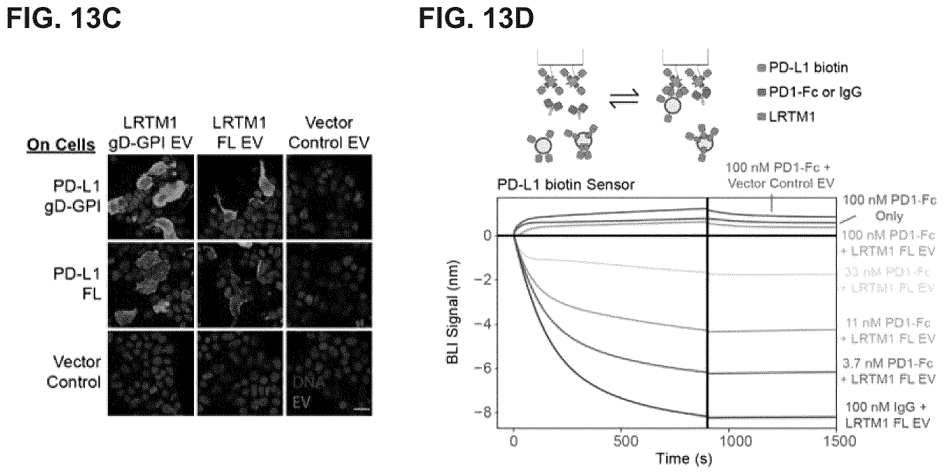

Next, to determine whether EVs are broadly applicable as platforms for receptor display, a variety of full-length, untagged, receptors were overexpressed in cells along with gag-Rluc. In all cases tested, the receptors were readily detectable in EVs using antibodies specific for the receptors (FIG. 4A). To test a larger set of receptors and measure their incorporation in EVs, detection methods not dependent on receptor-specific antibodies were needed. To address this, a collection of unrelated receptor ectodomains was fused to a glycoprotein D (gD)-glycosylphosphatidylinositol (GPI) tag. The GPI provides a lipid anchor that maintains ectodomains in membranes, while the gD epitope tag allows ectodomain detection using an anti-gD antibody. When these receptors were tested for incorporation into vesicles, most of the receptors assayed were readily detectable in EVs using an anti-gD antibody (FIGS. 4B, 4C, and 13A). Thus, the gD-GPI tagging strategy allows for easy detection of a variety of proteins in EVs.

C. Generation of Extracellular Vesicles Expressing Membrane Proteins

HEK293T or EXP1293™ cells were transiently transfected with a receptor of interest or an empty vector control (as indicated) and a plasmid expressing HIV gag fused to either Rluc (for screening) or mNeonGreen (for visualization). All plasmids transfected were cloned into the expression vector pRK5 (Genentech). For vesicle harvesting, cells were removed by either spinning at 300×g for 10 minutes followed by clearing at 2000×g for 20 minutes (for expressions <100 mL) or filtered out (for expressions ≥100 mL). For full library screens, rEV expression was done at a 1 L scale and grown for 7 days. cOmplete™ EDTA-free Protease Inhibitor Cocktail Tablets (Roche) were added as per specifications. After fully dissolving the tablets, the conditioned media was spun at 12,000×g for 40 minutes to remove any remaining dense particulates and microvesicles. The supernatants were transferred to 70 mL polycarbonate ultracentrifuge tubes (Beckman Coulter) at a volume of about 60 mL per tube. 10 mL of 50% sucrose were layered from the bottom of the tube using a syringe and long needle, forming a sucrose cushion. Samples were spun at 100,000×g for 90 minutes in a Ti-45 rotor. Vesicles float on top of the sucrose. Media above the vesicle layer was aspirated. Two tubes worth of cushion and vesicles were combined and diluted to 70 mL in new ultracentrifuge tubes using PBS. Samples were spun again at 100,000×g and the resulting pellet was dissolved in PBS. HALT™ Protease Inhibitor Cocktail (Thermo Fisher Scientific) was added to 1×. All spins were done at 4° C.

HEK293T cells were used to confirm that overexpressing receptors and gag protein fusions were detectable in vesicles. HEK293T were cultured in DMEM+GlutaMax supplemented with 10% FBS and penicillin/streptomycin (Thermo Fisher Scientific).

### Example 2. Identification of Binding Partners for PVR

A. Identification of Binding Partners for PVR Using EVs

To determine whether receptors expressed on EVs were accessible and binding competent, a proof-of-concept study was performed using the receptor PVR.

The identification of binding partners for a receptor-of-interest requires that the rEVs display accessible and binding-competent receptor on their surfaces. The poliovirus receptor (PVR) was used for proof-of-concept studies. PVR is a useful benchmark because it is known to bind to a variety of well-characterized cell surface-expressed receptors with different binding affinities (Husain et al., Mol. Cell. Proteomics, 18: 2310-2323, 2019). To determine whether PVR on rEVs was active, rEVs expressing full-length PVR were isolated and tested for binding to a number of PVR binding partners, expressed on the cell surface. PVR-rEVs selectively bound to cells expressing PVR ligands, with negligible background in untransfected cells under the conditions tested (FIG. 2E).

Next, to determine whether the gD-GPI strategy resulted in binding-competent receptor display, the PVR ectodomain was fused to a gD-GPI tag. gD-GPI vesicles were measured at a total protein concentration of 0.1 mg/mL in PBS against 10 μg/mL mouse anti-gD antibody (Abcam) using Anti-Mouse IgG Fc Capture Biosensors (ForteBio) by BLI. Similar to EVs expressing full-length PVR, PVR ectodomain-gD-GPI EVs selectively bound to cells expressing the PVR ligands, indicating that the tagged ectodomain was active for binding (FIG. 1F). The gD tag also enabled detection of PVR receptor expression on the EVs by electron microscopy (FIG. 4C). For general vesicle staining, the suspension of vesicles was adsorbed for 15 minutes to the surface of formvar- and carbon-coated TEM grids. After a short rinse with distilled water, samples were stained with 2% phosphotungstic acid (PTA) for 60 seconds and then air dried. For gD epitope detection, vesicles were adsorbed for 30 minutes and blocked with Aurion Blocking Solution for goat conjugates for 30 minutes. Samples were then stained with mouse anti-gD (abcam) for 1 hour in the blocking solution and detected using goat-anti mouse 12 nm gold conjugate. Samples were then washed with PBS for 15 minutes, washed with water for 1 minute, and stained with 1% uranyl acetate for 1 minute before being blotted and air dried. Imaging was done with a JEOL JEM-1400 transmission electron microscope (TEM) and a GATAN ULTRASCAN® 1000 CCD camera at magnifications from 5,000× to 50,000×.

B. Comparison of EV-Based Assays to Assays Using Recombinant Protein

Next, we determined whether rEVs can bind tightly enough to survive the washing steps and handling delays of our high-throughput assays. To this end, we determined the kinetics of binding for rEVs displaying either full-length PVR or the gD-GPI tagged ectodomain of PVR and for recombinant monomeric PVR to the known ligand CD226. Using a non-destructive technique, biolayer interferometry (BLI), a strong negative BLI signal was observed for both PVR rEV types, as expected given the large size of the rEVs (FIG. 2G, left panel). This was not observed when PVR-rEVs were incubated with a human IgG as a control, suggesting that this is a specific interaction (FIG. 2G, right panel). The rEVs showed little dissociation over ten minutes, making them suitable for high-throughput screening (FIG. 2G, right panel). Further, the EVs achieved faster association and slower dissociation relative to the PVR monomer, indicative of a higher affinity interaction (FIG. 2G, right panel). Notably, and consistent with the cell-based assays (FIGS. 2E and 2F), the gD-GPI tagged PVR EVs showed a similar binding profile to the full-length PVR EVs, further showing that the engineered receptor is amenable for detection of in-trans binding partners.

C. BLI Methods

BLI measurements were performed using an 8-channel OCTET® RED system (ForteBio). For PVR rEV binding, CD226-Fc (R&D SYSTEMS®) or native human IgG (abcam) was loaded at 25 nM onto Anti-Human IgG Fc Capture Biosensors (ForteBio). All recombinant proteins were loaded for 300 seconds. All measurements were performed at 30° C. Analysis was performed on the Octet® System Data Analysis software (ForteBio). A PBS buffer control was subtracted to account for drift in the instrument (unless PBS curve is shown). Alignment was to baseline and Savitzky-Golay filtering was performed. Values greater than 2 nm of association over 600 seconds are recommended.

D. EV Sample Characterization Methods

Total protein concentrations of the EV samples were measured by mixing 1.5 μL of the sample with 148.5 μL of Quick Start Bradford Protein Assay Reagent (Bio-Rad). The concentration was calculated against a titrated BSA curve. This reagent contains methanol, which permeabilizes membranes, so no detergents were added. EV particle numbers and concentrations were calculated from nanoparticle tracking analysis using NanoSight NTA (Malvern Panalytical). Vesicles at 0.1 mg/mL total protein were diluted 1000× in PBS and run for 5 repeats of 1-minute-long recordings using a 488 nm laser. Traces were analyzed using NTA 3.4 software, which provided a particle concentration that was used to calculate a molarity.

E. Discussion

This example demonstrates that both full-length, native proteins and gD-GPI tagged ectodomains of the receptors under study are incorporated in EVs and are amenable to EV-based binding studies. Anchoring ectodomains to EVs using gD-GPI tagging increases the capabilities of RDIMIS. First, it enables comparison of the interaction profiles of ectodomains and full-length proteins. Second, when studying unrelated receptors, the gD-GPI tag provides a common way to compare expression and localization across receptors, avoiding the need for often unavailable receptor-specific antibodies. Lastly, the gD-GPI tag can anchor non-membrane proteins, providing a way to study extracellular matrix or secreted factors in proximity to membranes.

Levels of the target of interest in EVs appear to depend primarily on the level of expression in parent cells. Therefore, for low-expressing proteins, optimization of experimental conditions may be required to achieve sufficient expression for efficient detection of binding partners.

### Example 3. RDIMIS Enables Identification of STM Protein Interactomes in High Throughput

A. Design of EVs and Ectodomain Library for High-Throughput Screen

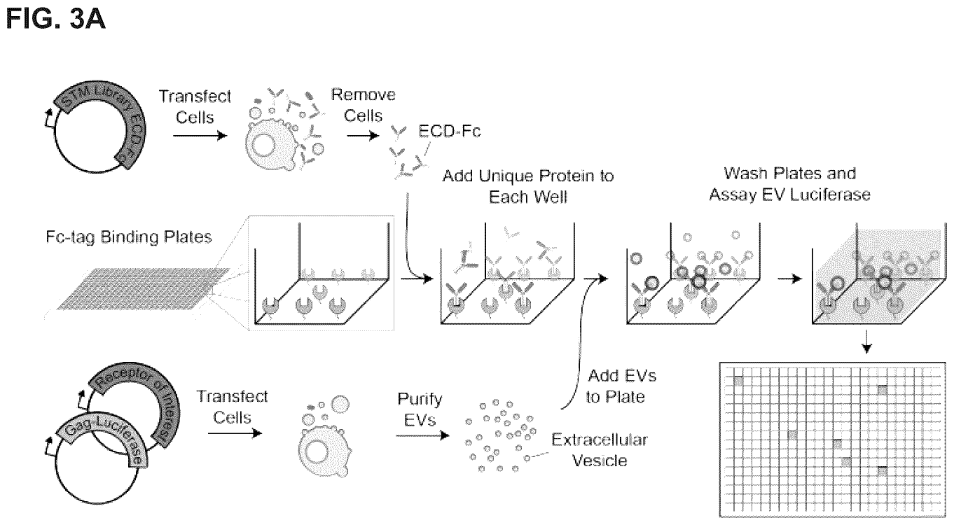

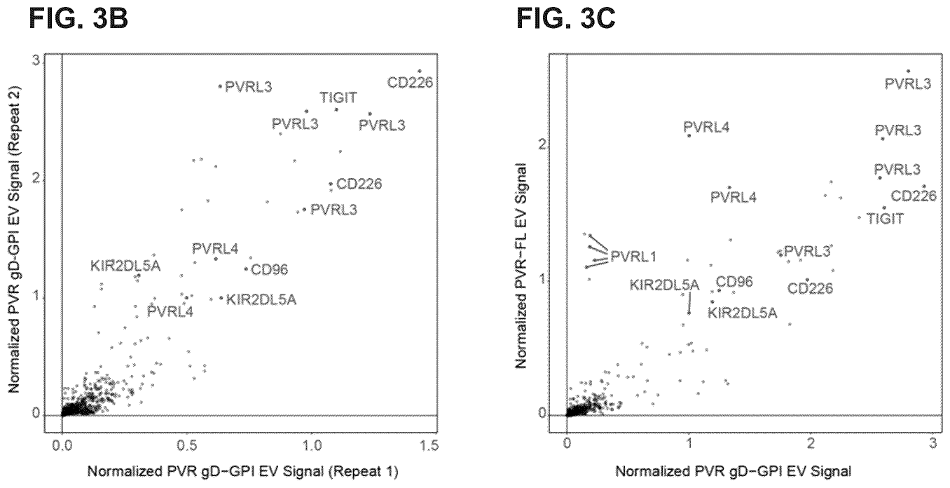

The RDIMIS system was next used for high-throughput discovery of receptor-ligand interactions (FIG. 3A).

To achieve unbiased identification, EVs expressing a protein of interest were assessed for binding with a previously developed conditioned media library consisting of most human single-pass transmembrane (STM) proteins expressed as ectodomain-Fc tag fusions (Martinez-Martin et al., Cell, 174(5): 1158-1171, 2018). To generate the library, cells were transfected with plasmids encoding the ectodomain-Fc tag fusions to induce them to express and secrete Fc-tagged ectodomains into the growth media. The media was then transferred to protein A-coated plates, with each well of the plate receiving a different Fc-tagged ectodomain. This resulted in a collection of immobilized ectodomains suitable for high throughput screening (Martinez-Martin et al., Cell, 174(5): 1158-1171, 2018).

In parallel, cell cultures for EV production were transiently transfected with plasmids encoding the receptor of interest and gag-Renilla luciferase (Rluc) (FIG. 3A). The receptor-containing EVs were isolated using an optimized purification protocol that enabled rapid large-scale isolation of EVs (FIG. 1A). After EVs were isolated, they were incubated with the plates containing the STM protein library. Plates were then thoroughly washed to remove unbound EVs. To detect interactions between the receptor-containing EVs and the STM proteins immobilized in the wells, Rluc substrate was added, thus generating a fluorescent signal in the wells where an interaction had taken place (FIG. 3A).

B. STM Interactome of PVR EVs

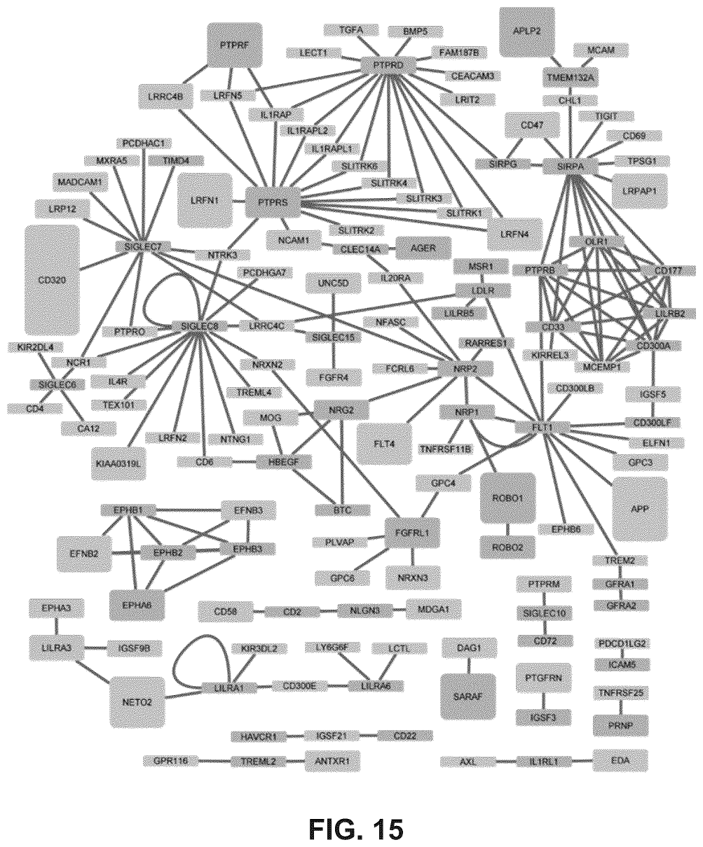

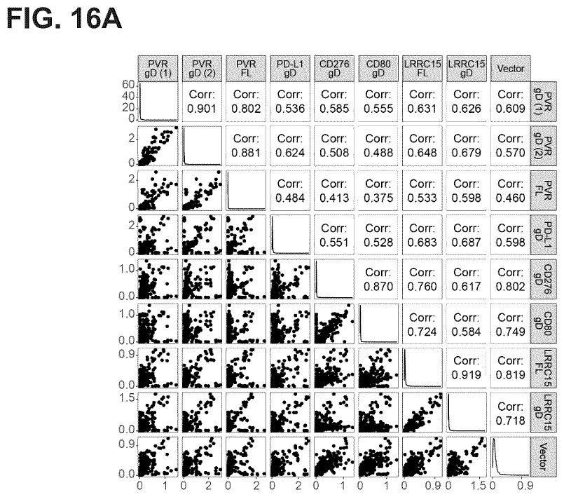

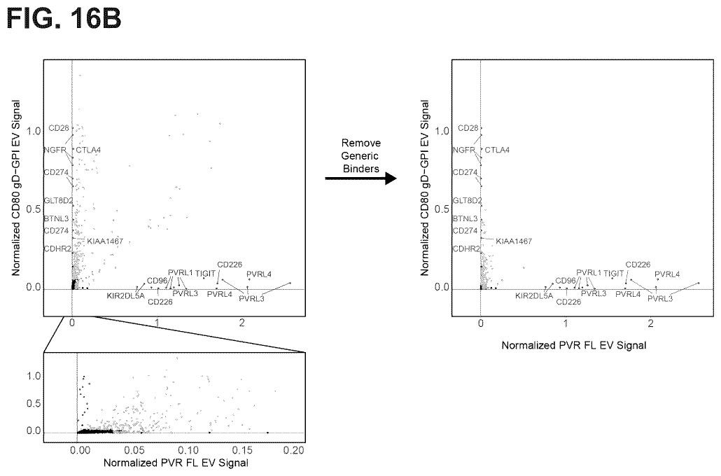

The RDIMIS platform was used to study the STM interactome of PVR-EVs (isolated EVs carrying a gD-GPI-tagged PVR ectodomain). Display of the receptor on the vesicles was confirmed using BLI and by Western blot, which showed robust binding of the rEVs to the gD antibody (FIGS. 4D and 13A). Notably, RDIMIS identified all expected PVR binding partners: CD96, CD226, KIR2DL5A, PVRL3, PVRL4, and TIGIT (FIG. 3B, blue) reproducibly across two independent rEV and STM library preparations (correlation coefficient of 0.90; FIG. 16A). These screens were performed using two independent PVR-EV preparations and two independent STM library preparations to control for potential variability during expression or preparation of the samples. Moreover, and importantly, the hits were virtually identical when RDIMIS was utilized to study rEVs carrying full-length, untagged, PVR (FIG. 3C), with an overall correlation coefficient of 0.88 (FIG. 16A). Together, these results further demonstrate that the expression of gD-GPI tagged receptors on rEVs allows detection of relevant ligands in-trans, which, in combination with the automated workflow developed, enables robust identification of membrane protein interactomes in an unbiased fashion and with enhanced sensitivity for detection of high and low affinity interactions.

C. STM Interactome of B7 Family Proteins

To further benchmark the sensitivity of this technique, the platform was applied to study three members of the B7 family of immunoregulatory proteins, which contains prominent immune receptors including the checkpoint inhibitor PD-L1. All three proteins were expressed as gD-GPI ectodomain fusions, allowing for their expression in vesicles to be monitored and directly compared with PVR gD-GPI containing rEVs. In all rEVs, the tagged ectodomains bound to an anti-gD antibody at levels comparable to PVR when assayed by BLI (FIG. 4D) or by Western blot (FIG. 13A).

a. STM Interactome of PD-L1 EVs

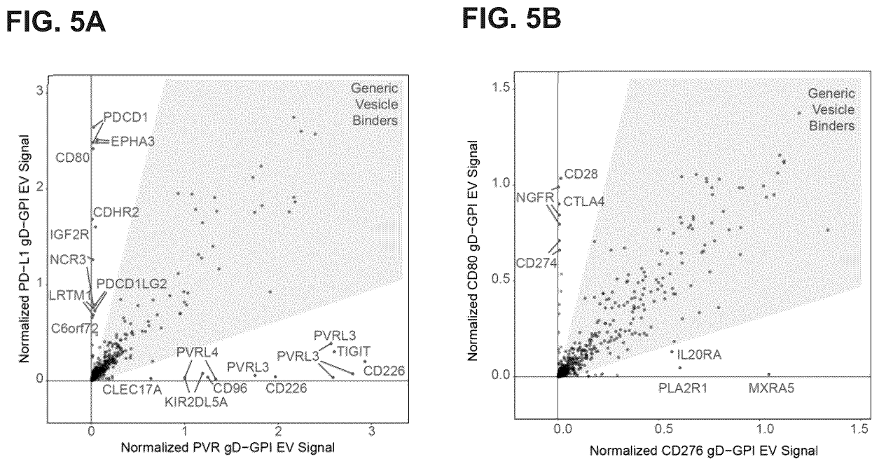

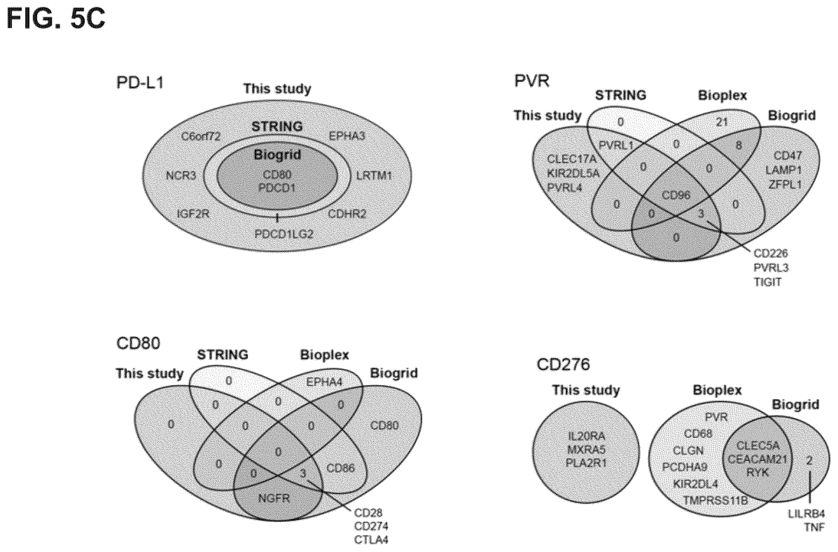

RDIMIS was applied to, PD-L1/CD274. PD-L1 was expressed as a gD-GPI-tagged ectodomain to allow for characterization of the PD-L1 EVs prior to screening, as described above. The PD-L1 EVs showed readily detectable binding to the gD antibody by BLI (FIG. 4D). When screened for interaction with the STM library, PD-L1 EVs identified the known PD-L1 ligands PDCD1 (PD1), EPHA3, CD80 (B7.1), and PDCD1 LG2 (PDL2) with high confidence. Since the estimated dissociation constant for the PD-L1/PD-L2 interaction is approximately 10 μM (Lee et al., Nat. Commun., 7: 1-9, 2016), this result further demonstrates that RDIMIS can identify biochemically challenging weak interactions. In addition, a number of other high-scoring hits were identified (FIG. 5A). While IGF2R has been found to be broadly sticky in unrelated experiments (Husain et al., Mol. Cell. Proteomics, 18: 2310-2323, 2019) and was thus labeled as non-specific interactor, the other hits represent new putative binders for PD-L1.

b. STM Interactome of CD80 and CD276 EVs

Finally, to further ensure the wide applicability of RDIMIS, the newly platform was applied to two additional membrane-expressed receptors, CD80 (B7-1) and CD276 (B7-H3) (FIG. 5B). Again, all relevant partners were detected for both proteins, confirming the broad utility of this methodology to cell surface-expressed targets. The well-described binders CD28, CTLA4, and PD-L1 and the more recently described binder NGFR were identified as the highest-scoring hits for the immune receptor CD80. In the case of B7-H3/CD276, deorphanized only recently through advances in screening technology (Husain et al., Mol. Cell. Proteomics, 18: 2310-2323, 2019), RDIMIS captured the recently described interactor IL20RA, as well as MXRA5, which was found to be a non-specific interactor in the same study (FIG. 5B). Notably, in both cases, the known interaction partners were among the highest-scoring hits, while several additional putative receptor-specific binding partners were identified that were previously not described in the literature, demonstrating the sensitivity of this method.

D. Comparison with Published Databases

Next, to get an overall sense of the number of the landscape of interactions identified for these four prominent immune receptors, the overlap between the receptor-specific hits identified using the RDIMIS method and interactions listed in the STRING (Szklarczyk et al., Nucleic Acids Res., 47: D607-D613, 2019), Bioplex (Huttlin et al., bioRxiv, doi:10.1101/2020.01.19.905109, 2020) and Biogrid (Oughtred et al., Nucleic Acids Res., 47: D529-D541, 2019) databases (FIG. 5C), some of the most comprehensive repositories for protein interactions, was assessed.

For an even comparison, only interactions between proteins that were present in the STM library queried in this study were considered. For the STRING database, only interactions designated as having experimental evidence were used. To generate a stringent list of hits for all screens, a cutoff was drawn at the 98% quantile for each screen because the distribution of the data deviated from a normal distribution and had a long upper tail. In all screens, a receptor-specific hit was called if signal in a particular screen was at least 4× that of the other screen.

As expected, most of the well-characterized interactions were represented in at least one of the databases, with eleven out of twelve interactions in STRING listed as having experimental evidence overlapping with our dataset (FIG. 5C). In addition, for all immune receptors under study, RDIMIS identified putative interactions that were not represented in publicly available databases (FIG. 5C). This suggests that identification of new interactions may be facilitated when the receptors of interest are studied in the context of the plasma membrane.

For CD80, the STRING database suggested an additional binding partner, CD86. Additional review of the literature, including the paper cited in support of this interaction in the STRING database, suggested these proteins do not appear to interact. While Bioplex and Biogrid do identify additional putative partners, these have largely not been validated.

E. RDIMIS Screen Methods

Vesicles were diluted into a final concentration of 0.03-0.05 mg/mL (as measured by Bradford) in 1×PBS+0.49 mM MgCl2+0.9 mM CaCl₂) (PCM)+1% BSA Fraction V (Sigma). Preparation of the human receptor library was performed using an integrated robotic system consisting of automated liquid handling devices (plate dispensers and washer) to allow for high throughput analysis of protein-protein interactions. Conditioned media containing Fc-tagged receptor ECDs were dispensed into white 384 well Protein A-coated plates (Thermo Fisher Scientific) and stored at 4° C. until needed. Concentration of the ECD-Fcs, varied but averaged 159 μg/mL. Plates were washed three times with PCM to remove unbound components of the conditioned media. Vesicles were added to the plates and allowed to sit overnight at 4° C. Plates were washed three times with PCM to remove unbound vesicles. To prevent drying, 25 μL of PCM was added to the plates. For a positive control used for normalization, 25 μL of the same vesicle stocks used in the screens were added into the first column of each plate after all washing steps. Since these are not washed, these positive control wells represent an input value, though due to automation limitations, they are diluted when compared to a well that is 100% bound. 25 μL of 1 μM coelanterazine h (Promega) in PCM is dispensed into the wells, incubated for 5 minutes, and then read on a TECAN using 0.1 s of luminescence read time. Since the majority of wells in the screens did not bind to any of the vesicles, additional negative control wells were not used in the analysis; rather, signals were analyzed with respect to the distribution of intensities across the whole screen to call hits. Empty wells tended to have higher signal than wells that had received ECD-Fc conditioned media that fell into the bottom half of the distribution and did not represent a non-binding Fc signal.

F. Quantification of EVs Using a Membrane Stain

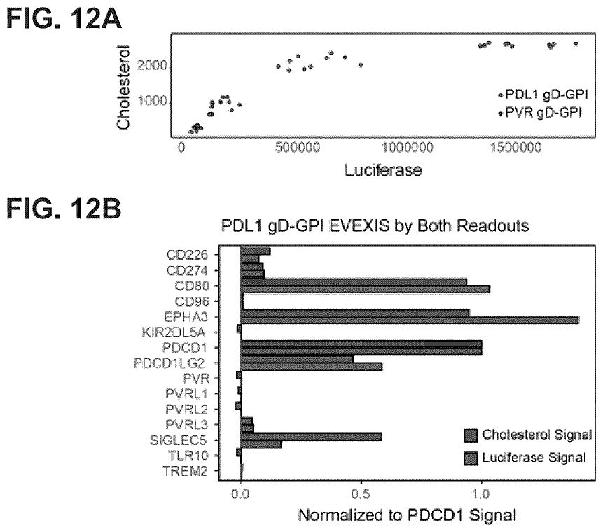

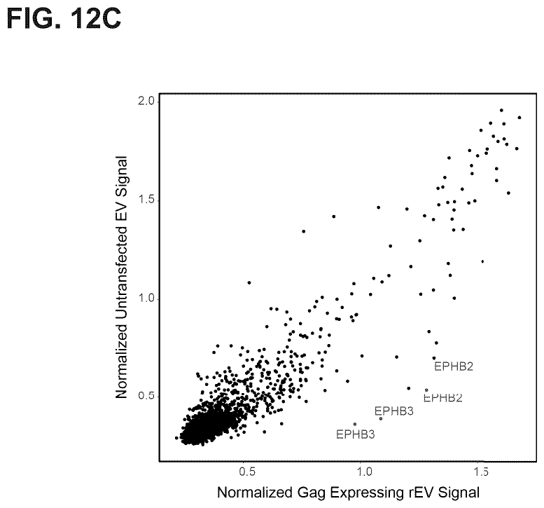

Cholesterol is a main component of the lipidic bilayer of the EVs. EVs were labeled with the AMPLEX™ Red Cholesterol Assay Kit (Thermo Fisher), thus enabling detection of EVs comprising non-tagged targets and/or endogenous EVs. A full RDIMIS screen was performed using either rEVs that were transfected with a vector control but contained gag-Rluc or EVs harvested from untransfected cells (FIG. 12C). Both EV species behaved similarly (correlation coefficient of 0.92), showing that rEVs and naturally generated EVs from the same cell line have a similar binding profile.

EVs were generated and harvested as described above. PD-L1 or PVR gD-GPI EVs containing gag-Rluc were serially diluted into a white 384 well plate, and EV concentration was measured using either Rluc by the addition of coelenterazine h (as in RDIMIS) or using the AMPLEX™ Red Cholesterol Assay Kit (Thermo Fisher) following the recommended protocol. To measure PD-L1 gD-GPI RDIMIS using the AMPLEX™ Red Cholesterol Assay Kit, EVs were allowed to bind as with normal RDIMIS. Plates were then aspirated dry, and 25 μL of the AMPLEX™ Red reagent was dispensed into each well. Stock EV solution was added to the first column of each plate to serve as a positive control. Plates were incubated for 30 minutes at 37° C. and read on a Tecan M1000 InfinitePro using excitation at 560+/1 10 nm and fluorescence detection at 590+/−10 nm (FIGS. 12A-12C).

Cholesterol esterase was included at the recommended amounts to ensure all cholesterol esters were also detected. Vesicles were incubated with Fc-ECD proteins as in the EVEXIS screen and washed as with EVEXIS. Rather than adding coelantrazine-h, plates were manually flicked dry. For the titration against luciferase signal, four 3× serial dilutions were made. 20 μL of 0.5 μM Coelantrazine h (Promega) was added, incubated for 5 minutes, and read on a Tecan M1000 InfinitePro. 20 μL of the Amplex Red Cholesterol Assay mix, as specified in the manual, was added to the wells, incubated for 1 hour, and read. Luminescence was read out on a Tecan M1000 InfinitePro using 0.1s of luminescence read time. Fluorescence was read out in a TECAN using an excitation of 560 nm and an emission wavelength of 590 nm. A blank well with just PBS and Coelantrazine h and Amplex Red Cholesterol Assay mix was also measured and the values subtracted from the signal. For PD-L1 gD-GPI EV binding, wells were flicked dry between the Coelantrazine h and Amplex Red Cholesterol Assay. Rather than subtracting a blank well, a well where no Fc-tagged species was transfected but conditioned media was still added, was used.

G. Interactions of LRTM1

To further assess the sensitivity of the assay, one of the new hits that had a low signal, LRTM1, was investigated. Since commercially available protein could not be found for LRTM1, LRTM1 expressed on rEVs was used to study the interaction. This study showed that PD-L1 ectodomains selectively bound to LRTM1 on rEVs presented as either a gD-GPI tagged ecotodomain or as the full-length protein (FIG. 13B). LRTM1 rEVs also selectively bound to gD-GPI tagged or full-length PD-L1 expressed on cells over cells transfected with a vector control (FIG. 13C). Since PD1 (PDCD1) is a known interaction partner of PD-L1 and the target of checkpoint blockade immunotherapy in cancer, experiments were performed to determine whether these interactions competed. Increasing concentrations of recombinant PD1-Fc protein were able to outcompete LRTM1 vesicle binding in a concentration-dependent manner (FIG. 13D), suggesting that the proteins bind to similar regions on PD-L1.

### Example 4. RDIMIS Enables Identification of Generic Extracellular Vesicle Binders

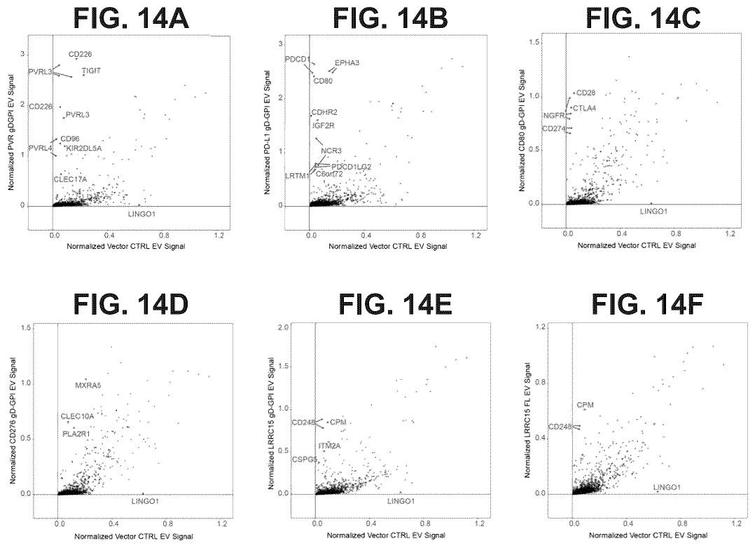

Interestingly, all unrelated screens revealed additional hits scattered among the expected binding partners (Table 8). As rEVs are complex mixtures, it was not clear whether these new binders were specific to the receptors expressed on the rEVs, or to the vesicles themselves. To address this, screens for rEVs displaying different receptors were directly compared to identify common and differential binders. Batch-matched screens were plotted against each other (FIGS. 5A and 5B) or against a screen with cells transfected with a vector control instead of a receptor-of-interest done independently (FIGS. 14A-14F). This revealed three distinct groups: PVR-specific binders (blue), PD-L1-specific binders (red), and a population of hits that were not enriched in a particular screen and therefore, were not receptor-specific (FIGS. 5A and 5B, gray shading; Tables 8 and 9).

Notably, several protein families were enriched in the generic vesicle binder list, including sugar binders like CLECs and SIGLECs, but also signaling receptors like LILRs and ephrin receptors. To determine whether any biological pathways or functions were overrepresented, gene ontology (GO) enrichment analysis was performed for the set of generic vesicle binders identified in the four RDIMIS screens shown in FIGS. 5A and 5B. Table 10 shows results of a GO enrichment analysis for the generic vesicle binders of Table 8, as performed using PANTHER15.0 overrepresentation test (2020 Mar. 23 release). Table 11 shows results of a GO enrichment analysis for the generic vesicle binders of Table 9, as performed using the PANTHER16.0 release overrepresentation test (Mi et al., Nucleic Acids Res, 49: D394-D403, 2021). The molecular functions significantly enriched included carbohydrate, sulfur and anion binding, all consistent with general binding to vesicles and cellular membranes. The list was also enriched in proteins associated with tertiary granule and tertiary granule membranes by a GO cellular component analysis (Table 12), further suggesting that these are general EV binders. GO biological process analysis was also performed but no significant results were found. Thus, this platform enables the identification of vesicle-specific binders that are conserved across interactomes for unrelated targets of interest, suggesting previously unknown receptors for the EVs.

TABLE 10
GO enrichment analysis for generic vesicle binders

| GO molecular function | raw P-value | FDR |
|---|---|---|
| carbohydrate derivative binding | 9.40E−09 | 1.02E−05 |
| anion binding | 1.82E−07 | 9.88E−05 |
| sialic acid binding | 3.65E−07 | 1.32E−04 |
| heparin binding | 2.59E−06 | 7.02E−04 |
| sulfur compound binding | 6.37E−06 | 1.38E−03 |
| carbohydrate binding | 6.48E−06 | 1.17E−03 |
| glycosaminoglycan binding | 6.14E−05 | 9.50E−03 |
| carboxylic acid binding | 8.90E−05 | 1.20E−02 |
| organic acid binding | 8.90E−05 | 1.07E−02 |
| axon guidance receptor activity | 1.47E−04 | 1.59E−02 |
| small molecule binding | 2.09E−04 | 2.06E−02 |

FDR: false discovery rate.

TABLE 11
GO enrichment analysis for generic vesicle binders

| GO molecular function | raw P-value | FDR |
|---|---|---|
| carbohydrate derivative binding | 1.20E−07 | 1.29E−04 |
| sialic acid binding | 2.10E−07 | 1.13E−04 |
| heparin binding | 6.57E−06 | 2.36E−03 |
| sulfur compound binding | 1.45E−05 | 2.60E−03 |
| carbohydrate binding | 8.41E−06 | .26E−03 |
| glycosaminoglycan binding | 7.87E−09 | 1.06E−02 |
| carboxylic acid binding | 8.63E−06 | 1.86E−03 |
| axon guidance receptor activity | 3.01E−04 | 2.94E−02 |
| transmembrane signaling receptor | 6.67E−05 | 1.03E−02 |
| activity | | |
| signaling receptor activity | 7.89E−05 | 9.44E−03 |
| molecular transducer activity | 7.89E−05 | 8.50E−03 |

FDR: false discovery rate.

TABLE 12
GO cellular component analysis for generic vesicle binders

| GO cellular component | raw P-value | FDR |
|---|---|---|
| tertiary granule membrane | 1.80E−05 | 1.28E−02 |
| tertiary granule | 7.25E−05 | 2.58E−02 |
| cytoplasmic vesicle | 2.38E−04 | 5.65E−02 |
| intracellular vesicle | 2.46E−04 | 4.38E−02 |

While the GO analysis suggested that many of the generic vesicle binders recognize common cell-surface modifications, many of the binders may also have protein interaction partners in the rEVs. To generate a list of potential binders, the generic vesicle binder list was cross-references with the published immunoglobulin superfamily receptome (Verschueren et al., Cell, 182: 329-344.e19, 2020) and the STRING database of interactions (Szklarczyk et al., Nucleic Acids Res., 47: D607-D613, 2019). Using these data, a network of potential interactions was generated (FIG. 15). Each node is a protein found in the network color-coded by whether it is a generic vesicle binder (green) or one identified from the published works and databases (blue). The source of the data is color-coded in the edges. To get a sense of whether these proteins are likely to be in the vesicles, expression data were cross-referenced for HEK293 cells, the parent cell line for the EXPI293™ cells used to generate the rEVs, using data from the Cell Atlas (Thul et al., Science, 356(6340), 2017. Based on the finding that the incorporation of a protein into the rEVs was typically correlated with its expression in the cells, it was reasoned that a highly expressed protein was more likely to be responsible for the binding that was detected. Expression is shown as the height of the boxes surrounding each protein name in FIG. 15, with taller boxes representing greater expression. This network suggests several highly expressed proteins that have known interactions with proteins in the generic vesicle binders list.

While high reproducibility was found for screens performed at the same time (PVR gD-GPI repeat 2 and PD-L1 gD-GPI; CD276 and CD80 gD-GPI; LRRC15 and PVR FL) or between cells expressing the same receptor (PVR gD-GPI repeats or PVR-FL), some variability was observed when all of the screens were plotted against each other (FIG. 16A). In particular, while some of the lower correlation was driven by receptor-specific hits (i.e. PVR gD-GPI vs. PD-L1 gD-GPI), the screens with the worst correlation were the CD80 gD-GPI and PVR FL screens. Interestingly, that this was driven by two separable populations of generic vesicle binders, one that seemed to be consistent between the screens (FIG. 16B, sky blue dots) and one that was enriched in the CD80 gD-GPI screen (FIG. 16B, gold dots). However, both populations could still be distinguished from CD80 gD-GPI specific hits, as those hits were significantly more enriched in the CD80 gD-GPI screen. This could be seen by either zooming in near the y-axis (FIG. 161a) or by removing the list of generic vesicle binders common between all of the screens (FIG. 1-60). To determine whether was some difference between these two populations, GO molecular functions analysis was performed on the two populations identified in this screen (Tables 13 and 14). Interestingly, heparin and glycosaminoglycan binding was enriched in the first population of generic vesicle binders (Table 13), while sialic acid binding and transmembrane signaling activity was enriched in the second population (Table 14).

TABLE 13
CD80 gD-GPI enriched generic binders

| GO Molecular Function | Raw p-value | FDR |
|---|---|---|
| Heparin binding | 4.52E−08 | 4.87E−05 |
| Sulfur compound binding | 1.42E−07 | 7.62E−05 |
| Glycosaminoglycan binding | 3.78E−06 | 1.36E−03 |
| Carbohydrate derivative binding | 1.75E−04 | 4.71E−04 |

TABLE 14
PVR FL enriched generic binders

| GO Molecular Function | Raw p-value | FDR |
|---|---|---|
| Sialic acid binding | 4.58E−07 | 4.93E−04 |
| Anion binding | 3.98E−06 | 2.14E−03 |
| Transmembrane-ephrin receptor | 5.21E−06 | 1.87E−03 |
| activity | | |
| Carbohydrate derivative binding | 5.90E−06 | 1.59E−03 |
| Ephrin receptor activity | 9.69E−06 | 2.09E−03 |
| Carboxylic acid binding | 1.10E−05 | 1.98E−03 |
| Carbohydrate binding | 5.29E−05 | 8.14E−03 |
| Transmembrane receptor | 3.64E−04 | 4.90E−02 |
| protein tyrosine kinase activity | | |

To get the list for each individual pair of screens, a cutoff was drawn at the 90% quantile because the distribution of the data deviated from a normal distribution and had a long upper tail. This was done for each screen individually and the final list was the list of genes that was common between all screens which removed screen-specific hits. To rank the genes, the intensity was averaged across all screens and sorted by that average. Gene Ontology analysis was done using the PANTHER15.0 release overrepresentation test (2020 Mar. 23 release) or the PANTHER16.0 release overrepresentation test (Mi et al., Nucleic Acids Res, 49: D394-D403, 2021) on the list of generic vesicle binders. The reference list was a list of all genes in the STM library. GO molecular function complete, biological process complete and cellular component complete annotation data sets were analyzed using the Fisher's exact test.

The RDIMIS approach thus allows profiling of vesicle-specific binders, e.g., for the study of human EV biology. More than 100 binders for HEK cell-derived, human EVs were identified, suggesting putative receptors that may mediate EV interactions with cells, an aspect of vesicle biology that has remained elusive due to the lack of optimal tools (Gonda et al., Mol. Cancer Res., 17: 337-347, 2019). Our results indicate that EVs interact with major signaling proteins such as immunomodulatory proteins (e.g. SIGLEC family, LILR family and CD300 family), growth regulator proteins (e.g. growth factor receptors like FGFR4, FLT1 and NRP proteins and several receptor-tyrosine kinases) and neuronal proteins (e.g. APP and CLSTN proteins), supporting the idea that EVs mediate intercellular communication. Given the throughput and reproducibility, this method provides a way to assess the quality of EVs on the global scale. Further, this platform provides a unique tool to elucidate the players that influence EV functions at the molecular level; therefore, it can be used to detect unexpected changes in the binding profile of EVs arising from the overexpression of target receptors, addition of therapeutics, or modifications of the cells generating the EVs.

### Example 5. RDIMIS Identifies CD248 as a Novel Interaction Partner for the Orphan Cancer-Relevant Receptor LRRC15

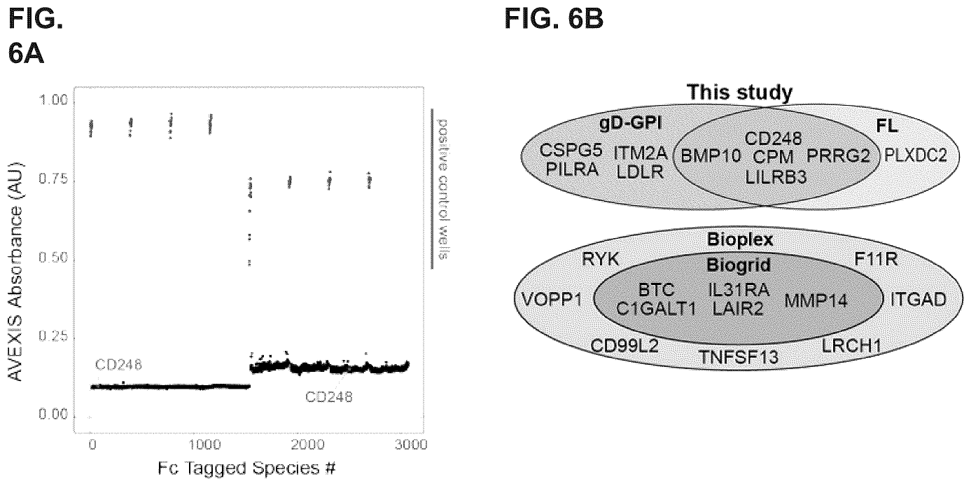

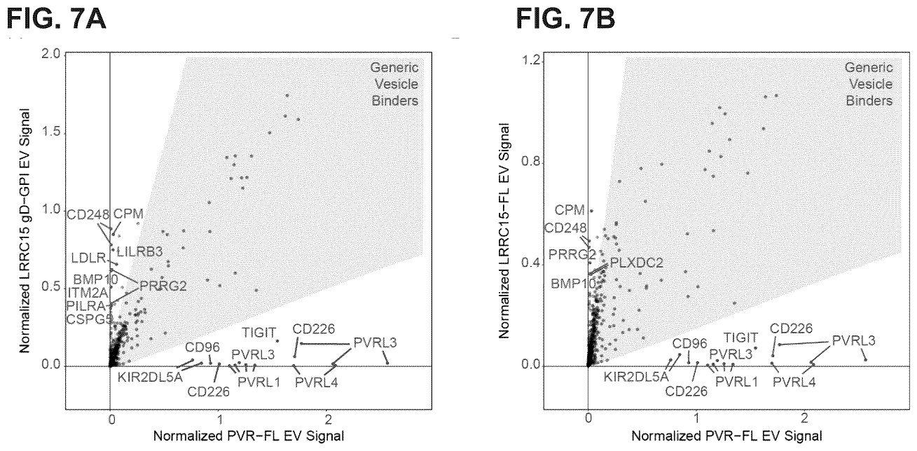

The above-described results suggested that RDIMIS may enable deorphanization of challenging targets refractory to other biochemical screening approaches. As an example, RDIMIS was used to study the cancer-associated fibroblast (CAF) receptor LRRC15. LRRC15 recently emerged as a specific marker for CAFs associated with large tumors (Dominguez et al., Cancer Discov., 10(2): 232-253, 2020). Despite this biological importance, no interaction partners have been identified, and thus basic aspects of LRRC15 biology remain undefined. LRRC15 interaction partners were first searched for using a previously implemented technology, miniaturized AVEXIS (Martinez-Martin et al., Cell, 174(5): 1158-1171, 2018; Bushell et al., Genome Res., 18: 622-630, 2008). This technology screens pentamerized LRRC15 ectodomain against the STM protein library described above. Despite the high sensitivity of the AVEXIS screen for detection of transient interactions previously shown with this technology, no binding partners were identified for LRRC15 (FIG. 6A), suggesting that LRRC15 may require a more physiologically relevant setting for optimal activity. To test this hypothesis, LRRC15 was screened as gD-GPI (FIG. 7A) and full-length (FIG. 7B) receptor-rEVs using RDIMIS. Notably, both of these efforts identified similar sets of putative interactors for LRRC15, which were not described in available databases (FIG. 6B).

CD248 was biotinylated using EZ-LINK™ Sulfo NHS-LC-LC-Biotin (Thermo Fisher) and cleaned up on a Zeba desalting column with a 7K MWCO (Thermo Fisher). CD248 was loaded onto Streptavidin (SA) Biosensors at 25 nM. LRRC15-Fc protein (Genentech) was provided at 500 nM. LRRC15 rEVs were provided at 0.25 mg/mL total protein (2.5-3.5 nM).

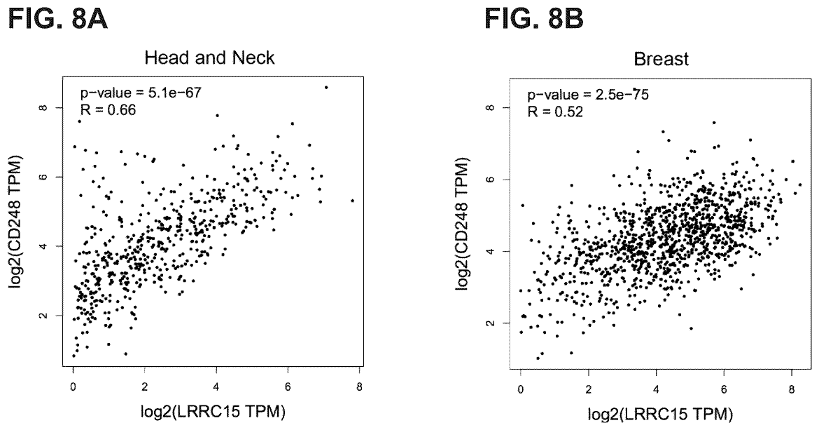

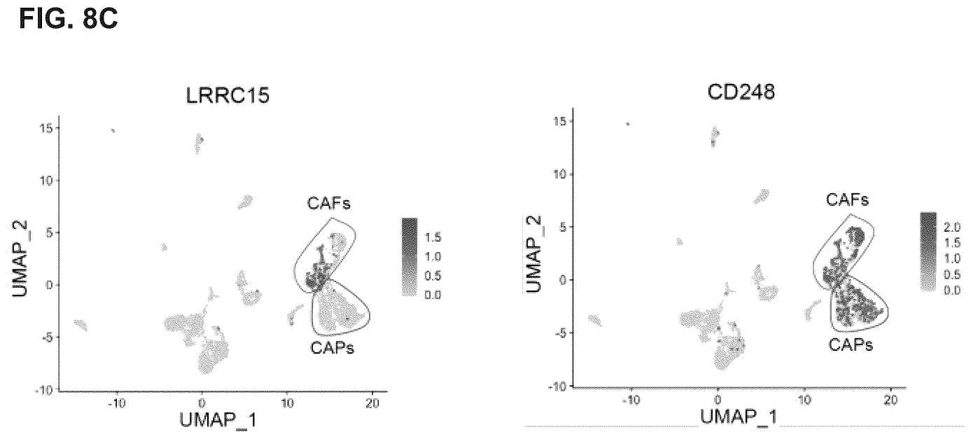

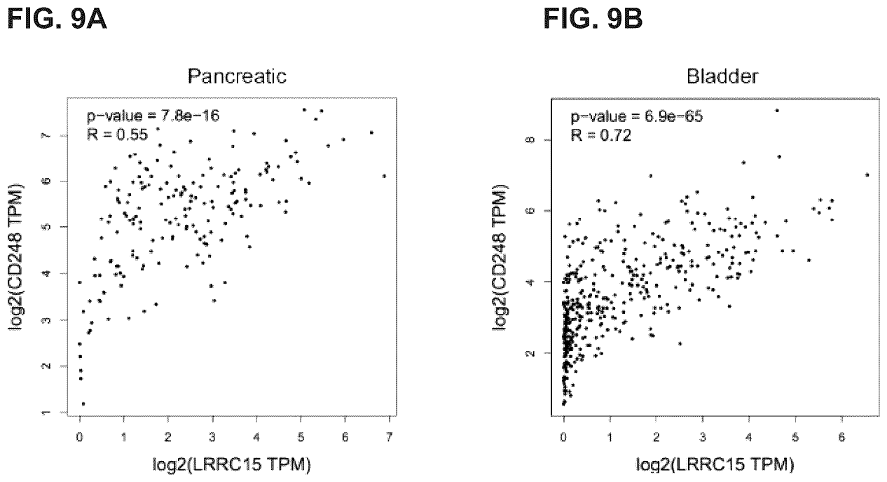

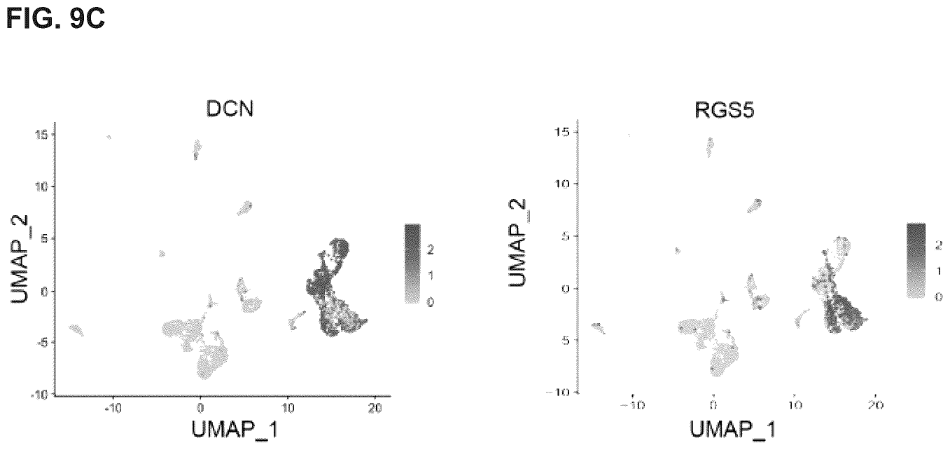

Since CD248 was a top scoring hits in both screens, its expression is upregulated in tumor stroma (Rouleau et al., Clin. Cancer Res., 14: 7223-7236, 2008; Rouleau et al., Int. J. Oncol., 39: 73-89, 2011; Teicher et al., 10: 993-100, 2019), and it has been suggested to promote tumor growth (Maia et al., BMC Cancer, 2: 1-12, 2011), the LRRC15-CD248 interaction was selected for further characterization. While LRRC15 and CD248 have been independently reported to be upregulated in solid tumors (Rouleau et al., Clin. Cancer Res., 14: 7223-7236, 2008; Purcell et al., Cancer Res., 78: 4059-4072, 2018), it was unclear whether they were expressed in the same tumor samples. Significant correlations between the expression of CD248 and LRRC15 (FIGS. 8A, 8B, 9A, and 9B) were identified in bulk RNA-seq data from The Cancer Genome Atlas (TCGA) for four different tumor indications. These correlations suggest that CD248 and LRRC15 are either found on the same cell type or are co-regulated. To distinguish between these two possibilities, single-cell RNA-seq data from head and neck cancer patients were re-analyzed to highlight LRRC15 and CD248 expression (Puram et al., Cell, 171: 1611-1624e.24, 2017). This analysis revealed that LRRC15 and CD248 are co-expressed on a subset of CAFs (co-occurrence score (Odds ratio)=9.44) (FIG. 8C), with CD248 showing a broader expression that encompasses all CAF and cancer-associated pericyte (CAP) cells identified using markers such as DCN and RGS5 (FIG. 9C).

Together, these results position RDIMIS as a robust method to identify new interactors for receptors not amenable to other technologies that rely on recombinant protein expression. Further, while it is unclear whether the interaction between LRRC15 and CD248 is occurring on the same cell or between cells, the above analysis suggests that these proteins have ample opportunity to interact in patient tumors, providing a potential biological context where this interaction might be relevant.

A. The LRRC15-CD248 Interaction Requires LRRC15 Expression on a Membrane

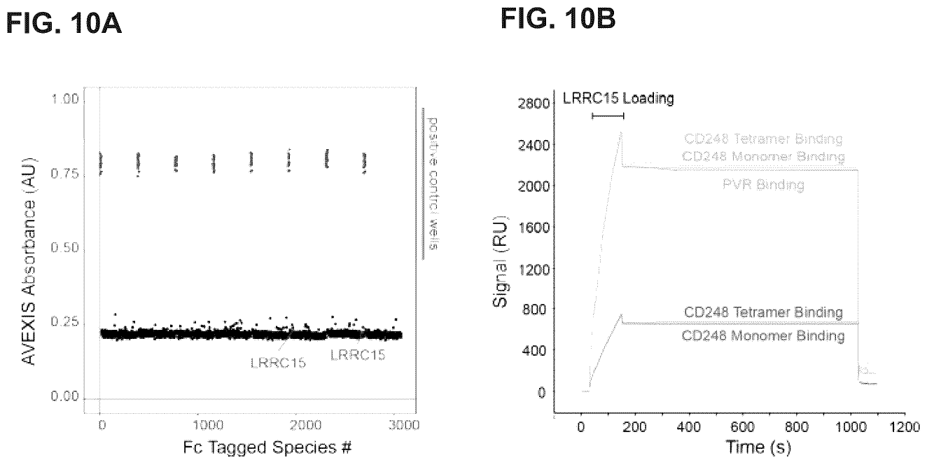

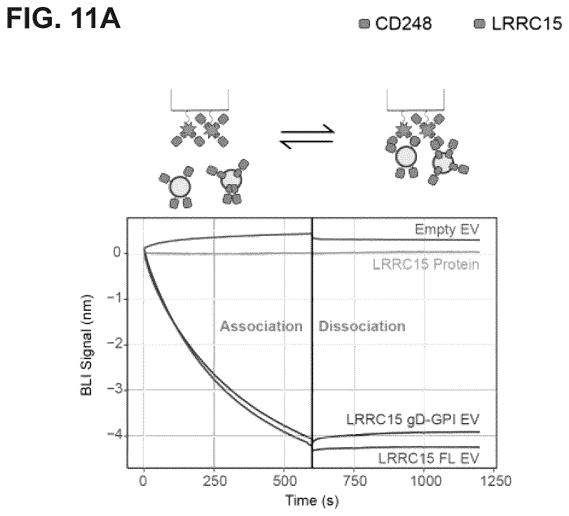

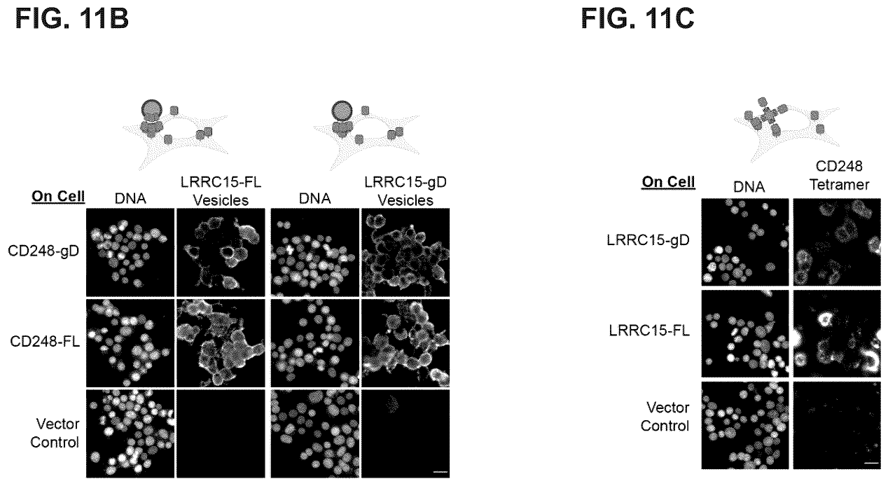

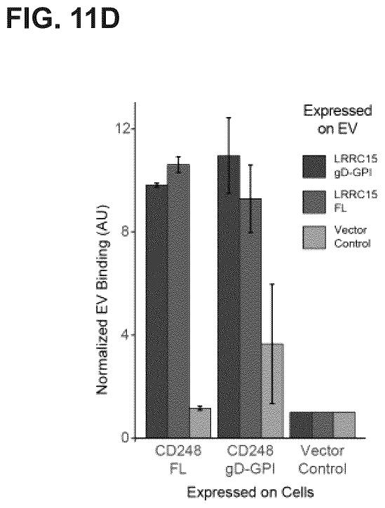

The LRRC15-CD248 interaction was further characterized using biophysical and biochemical methods. First, a miniature AVEXIS assay was performed using CD248 pentamerized ectodomains. Similar to LRRC15 (FIG. 6A), no high-confidence hits were identified when CD248 pentamerized ectodomains were screened against the STM protein library (FIG. 10A). Consistent with this result, no binding was observed between LRRC15 and CD248 recombinant proteins when the interaction was analyzed by either BLI or surface plasmon resonance, even when experimental conditions to maximize sensitivity of detection were employed (FIGS. 10B and 11A). BLI analysis confirmed lack of detectable binding when both LRRC15 and CD248 were tested as recombinant ectodomains. In contrast, the interaction was readily detectable when LRRC15 was displayed on EVs (FIG. 11A). Similarly, this interaction could also be observed on the plasma membrane of a cell. First, rEVs displaying LRRC15 bound to CD248 overexpressed on the cell surface (FIG. 11B) over ten times more than cells transfected with an empty vector control (FIG. 11D). Second, LRRC15 was expressed on cells and incubated with CD248 recombinant protein that was biotinylated and tetramerized using fluorescently labeled streptavidin. Binding was readily detectable for LRRC15-expressing cells, but not for cells expressing a control protein (FIGS. 11B-11D). These assays reinforce the notion that this interaction requires a membrane, but not specifically rEVs.

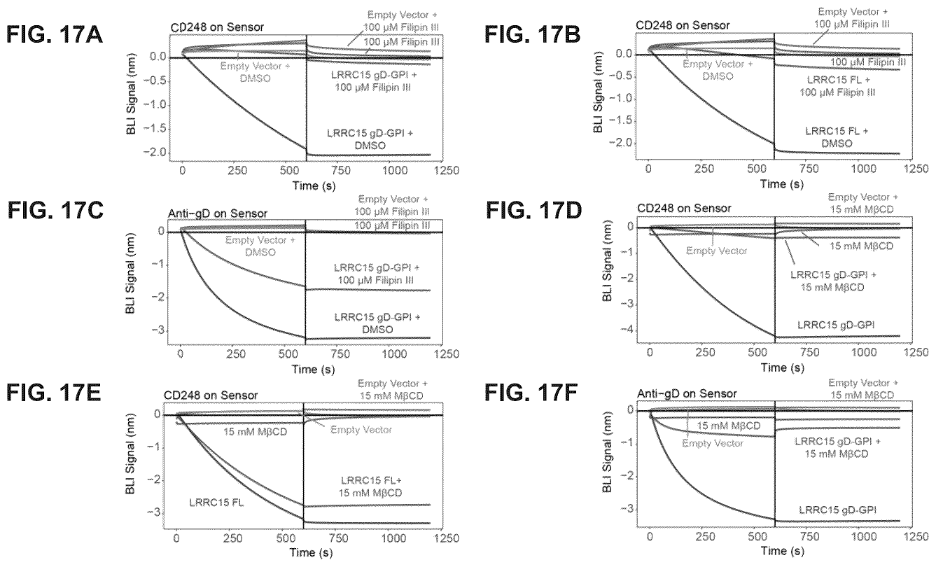

To better understand what might be underlying the membrane dependence of this interaction, the rEV membranes were altered either by disrupting them using Filipin III, which forms cholesterol ultrastructures (FIGS. 17A-17C) or by depleting membrane cholesterol with methyl-β-cyclodextrin (MβCD) (FIGS. 17D-17F) (Petro et al., Toxicon, 48: 1035-1045, 2006). That treating cells with 100 μM Filipin III or 15 mM MpβCD all but eliminated the binding of LRRC15 gD-GPI expressing vesicles to CD248 monomer as shown by BLI. These agents also affected the detection of the gD epitope tag by an anti-gD antibody. Interestingly, while 100 μM Filipin III also dramatically reduced the binding of LRRC15 FL rEVs to CD248, a minor effect was observed with MpβCD.

Though it was shown that LRRC15 and CD248 can interact in a membrane-dependent manner, it was not known whether they were present in the same physiological environment. Whereas LRRC15 and CD248 have been independently reported to be upregulated in solid tumors (Rouleau et al., Clin. Cancer Res. 14: 7223-7236, 2008; Purcell et al., Cancer Res., 78: 4059-4072, 2018), it was unclear whether they were expressed in the same tumor samples. Bulk RNA-seq data from The Cancer Genome Atlas (TCGA) for four different tumor indications showed significant correlations between the expression of CD248 and LRRC15 (FIGS. 8A, 8B, and 9A-9C). This correlation suggested that CD248 and LRRC15 are either found on the same cell type or that CD248 and LRRC15 are co-regulated. To help answer that question, single-cell RNA-seq data from head and neck cancer patients was re-analyzed to highlight LRRC15 and CD248 expression (Puram et al., Cell, 171: 1611-1624.e24, 2017). This revealed that LRRC15 and CD248 are co-expressed on a subset of CAFs (co-occurrence score (Odds ratio)=9.44) (FIG. 8C), with CD248 showing a broader expression that encompasses all CAF and cancer-associated pericytes (CAPs) identified using markers such as DCN and RGS5 (FIG. 9C). There are several models that may explain the membrane dependence of the interaction. The simplest model is that the LRRC15 ectodomain requires a membrane environment for proper folding. Interestingly, since both the full-length and gD-GPI-tagged ectodomain captured this interaction, the determinants responsible for this dependence may not be within the transmembrane domain or a precise spacing between the membrane and the ectodomain. Another plausible explanation is that the presence of the membrane promotes the formation of highly clustered arrays of receptors, increasing protein avidity beyond the tested pentamerization, to stabilize the interaction. This is in part supported by the evidence that Filipin III and MβCD can disrupt this interaction. In particular, Filipin III, which is thought to bind to but not remove hydroxysterols like cholesterol in the membrane (Bolard, BBA-Rev. Biomembr., 864: 257-304, 1986), could be reducing the ability of receptors to cluster. While the Filipin III could be causing a general disruption to the membrane and therefore affecting both the full-length and gD-GPI tagged species, the effect of MpβCD was primarily on the gD-GPI tagged LRRC15. This is consistent with works suggesting that GPI-anchored receptors like the folate receptor depend on membrane cholesterol for clustering (Rothberg et al., J. Cell Biol., 111: 2931-2938, 1990). Alternatively, the LRRC15-CD248 interaction may depend on the recruitment of a yet unknown factor that promotes or stabilizes the complex.

For immunofluorescence assays, HEK293T cells were split into 96-well SENSOPLATES™ (Greiner Bio-One) coated with 0.1 mg/mL Poly-D-Lysine (Gibco) for 30 minutes at 37° C. Cells were transfected using LTX Reagent (Thermo Fisher Scientific) according to the manufacturer's specifications. For fluorescent rEV experiments, rEVs were harvested from EXP1293F™ cells transiently co-transfected with gag-mNeonGreen and the receptor of interest. They were purified by ultracentrifugation at 100,000×g for 90 minutes after a 10 minute spin at 300×g and a 1 hr spin at 3,000×g to remove cells and debris. rEVs were resuspended in PBS and were incubated with cells for 30 minutes at 4° C. Cells were washed with PBS and fixed using 4% PFA for 10 minutes. CD248 protein (R&D Systems) was biotinylated using EZ-LINK™ Sulfo NHS-LC-LC-Biotin (Thermo Fisher Scientific), cleaned up on a Zeba 7K MWCO desalting column, and tetramerized using streptavidin-APC (Agilent). DNA was stained with 10 μg/mL Hoechst 33342 (Tocris Bioscience).

Altogether, these results confirm CD248 as a new interaction partner for LRRC15, and indicate that the LRRC15-CD248 interaction is facilitated in the context of a membrane. These findings highlight the advantages of RDIMIS, which provides increased performance for detection of interactions that involve membrane proteins, and that are refractory to other technologies that necessitate the use of recombinant proteins.

Beyond identification of protein interactomes, the rEV-based receptor-display can provide quantitative explorations of receptor behavior in membranes. For example, BLI can be used to characterize the binding kinetics of a receptor with its ligand in a membrane (FIG. 11A).

### Example 6. G Protein-Coupled Receptor Interaction Screening Enables the Discovery of a Novel Receptor for PD-L1 (Programmed Cell Death Ligand 1) Called Adhesion GPCR B1 (ADGRB1)

A. Background

The G protein-coupled receptor (GPCR) superfamily encompasses nearly 20% of the extracellular proteins in the human genome and is the target of over one third of all FDA approved drugs. However, only two biological drugs, which normally disrupt extracellular protein interactions, target GPCRs. GPCRs have lagged behind in biological drug development partly because extracellular interaction mapping of GPCRs has lagged behind the rest of the human genome. Therefore, a GPCR-focused extracellular interaction screen was implemented to map the interactions of receptors targeted for immunotherapies in cancer. A novel receptor for PD-L1 (programmed cell death ligand 1) called adhesion GPCR B1 (ADGRB1) was observed. Secondly, the interactions of ADGRB1 were mapped against the majority of the single-transmembrane receptors in the human genome, and a novel interaction with ICOSLG (inducible T cell costimulatory ligand) was observed. These data demonstrate the potential for GPCR interaction screening in mapping new biology and developing new avenues for therapeutic interventions in cancer.

The field of interaction mapping, often referred to as “interactomics,” was developed in order to understand cellular protein/protein interactions. Beginning with the development of the yeast-two-hybrid screen in 1989 (Young, Biology of Reproduction, 58(2): 302-311, 1998), interaction mapping has yielded many insights into how protein interaction networks function under physiological conditions, and in disease. Recently, the field of interactomics has exploded with thorough mapping of context-specific protein networks (Go et al., Nature, 595: 120-124, 2021; Huttlin et al., Cell, 184(11): 3022-3040, 2021). One of the major challenges in mapping protein/protein interactions is to include proteins embedded in cellular membranes. Defining interaction networks in the extracellular space is even more challenging, due to the typically transient interactions that often involve posttranslational modifications, such as cysteine reduction and glycosylation (Martinez-Martin, J. Immunol. Res, 2017: 2197615, 2017). Therefore, extracellular protein interaction mapping has lagged behind in the interactomics field, as new technologies have been required to drive the field forward.

One of the key insights into developing extracellular interaction technologies was the recognition that avidity is a critical component to deciphering transient, but physiologically relevant, extracellular protein interactions (Gonzalez, Methods, 57(4): 448-458, 2012). Screens have been developed to utilize avidity to map interactions across most of the extracellular proteins in the human genome. For example, the discovery that the poliovirus receptor (PVR) interacts with TIGIT (T cell immunoglobulin and ITIM domain) was made by screening potential proteins as Fc-tagged dimers (Yu et al., Nature Immunology, 10(1): 48-57, 2009). Microbeads have been used to multimerize ligands and screen against protein microarrays to map the extracellular interactome of the human adenovirus (Martinez-Martin et al., Nature Communications, 7: 11473, 2016). Similarly, baculovirus was used as a way to present receptors as a multimerized probe for screening against these protein microarrays (Tom et al., Analytical Biochemistry, 479: 1-5, 2015). Large-scale libraries of Fc-tagged extracellular proteins have been used to screen for interactions using microbeads (Husain et al., Mol. Cell. Proteomics, 18: 2310-2323, 2019) and also ligands that were multimerized by genetic fusion for avidity-based extracellular interaction screening (Martinez-Martin et al., Cell, 174(5): 1158-1171, 2018; Verschueren et al., Cell, 182: 329-344.e19, 2020). However, despite these successes, one major family of extracellular proteins has remained intractable in interactomic screens: multi-transmembrane receptors (MTMRs).

The human genome encodes more than 5,000 receptors and secreted proteins that interact with the extracellular space. Single-transmembrane receptors encompass over 2,000 of these extracellular proteins, while secreted proteins represent over 600 (Uhlén et al., Science Signaling, 12(609), 2019). The remaining less than 2,000 extracellular proteins are MTMRs and over 800 of those belong to the G protein-coupled receptor (GPCR) superfamily. The GPCR superfamily has a rich history of successful clinical therapeutics developed against it, with one third of all FDA approved drugs targeting just over 100 of these 800 GPCRs (Congreve et al., Cell, 181(1): 81-91, 2020). The remaining members of the GPCR superfamily include over 400 olfactory receptors, although these receptors have mostly restricted expression in the olfactory epithelium. However, olfactory receptors should not be excluded from consideration for drug development as they have been shown to be important drivers of physiology and dysregulated in disease models in tissues where the olfactory receptors are expressed outside of the olfactory epithelium (Pronin and Slepak, The Journal of Biological Chemistry, 296: 100475, 2021). The GPCR superfamily is vital in almost every physiological system and is such a successful drug target because the receptors are expressed at the plasma membrane but their expression is relatively low and restricted to specific cell types.

While the plasma membrane localization and low and restricted expression of the GPCR superfamily make these proteins ideal targets for drug development, these same properties present unique challenges for studying GPCR biology. Indeed, there are still over 100 orphan GPCRs with unknown ligands that control critical physiological functions (Laschet et al., Biochemical Pharmacology, 153: 62-74, 2018). Additionally, interaction mapping of GPCRs has lagged behind other extracellular proteins (Dunn et al., Pharmacological Reviews, 71(4): 503-519, 2019). GPCRs are also underrepresented in biological drug development. While GPCRs encompass over 30% of FDA-approved drugs, the GPCR superfamily is only the target of 2% of the FDA approved biological drugs (Hutchings, Expert Opinion on Biological Therapy, 20(8); 925-935, 2020. Biological drug development allows for robust protein interaction disruption or enhancement, tissue targeting, and conjugated drug delivery (Lu et al., Journal of Biomedical Science, 27(1): 1, 2020). Secondly, it is possible to develop complex pharmacophores to virtually any allosteric site that is available as an antigen; this potential could speed the development of next generation GPCR drug classes, such as biased agonists or allosteric modulators. Therefore, a set of GPCR-focused tools that would allow for interaction mapping and biological drug characterization were built.

The first GPCR-focused platform described herein is a cellular overexpression system that utilizes ligand multimerization to detect protein/protein interactions on the cell surface. Secondly, GPCRs were packaged into recombinant extracellular vesicles (Geeurickx et al., Nat. Commun., 10: 1-12, 2019) and screened for interactions against the recently published library of Fc-tagged single-transmembrane receptor extracellular domains (Verschueren et al., Cell, 182: 329-344.e19, 2020). Using these complementary approaches, orphan ligands can be screened across all of the GPCRs in the human genome and orphan GPCRs can be screened against most of the proteins in the extracellular space.

B. Employment of a Comprehensive Cell-Based Overexpression Interaction Screen Reveals Adhesion Receptor B1 (ADGRB1) is a Novel Receptor for PD-L1 (Programmed Cell Death Ligand 1)

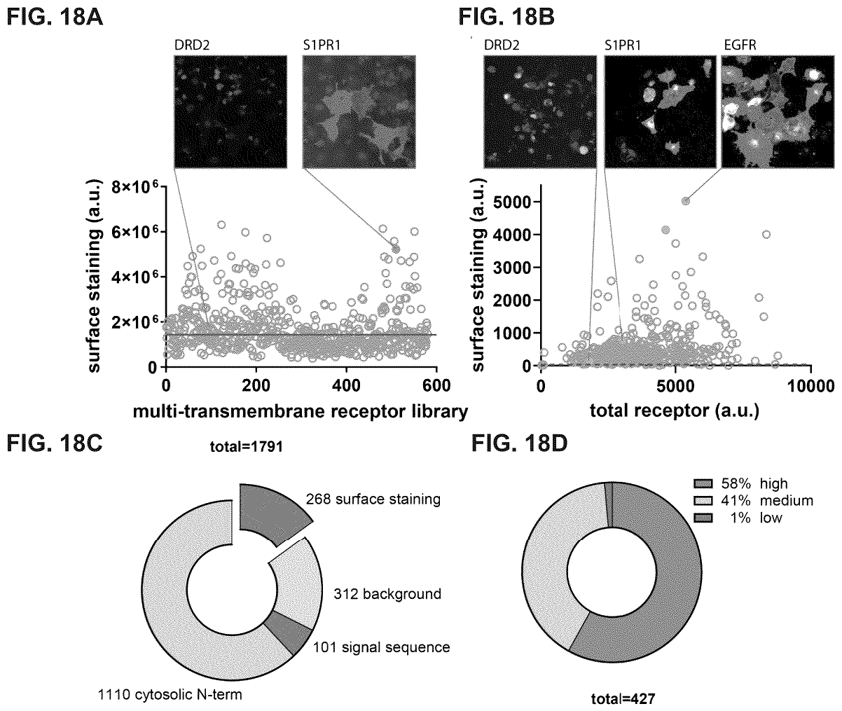

A comprehensive library of multi-transmembrane receptors in mammalian overexpression vectors was developed in collaboration with the DNASU plasmid repository (Seiler et al., Nucleic Acids Research, 42 (Database issue), D1253-1260) to clone their collection of multi-transmembrane receptors in gateway donor vectors into the pT-Rex-DEST31 plasmid (Invitrogen). This created an N-terminal HIS tag that was detected on the surface of cells (FIG. 18A). In addition, a G protein-coupled receptor-focused DNA library was created, with an N-terminal FLAG tag and C-terminal Venus. The GPCR collection allowed for the detection of low-expressing receptors (FIG. 18B). In all, the HIS-tagged MTMR collection allows for mostly unlabeled receptors to be overexpressed at the cell surface (FIG. 18C), while the GPCR-Venus collection allows for detection of low-expressing receptors above background staining (FIG. 18D).

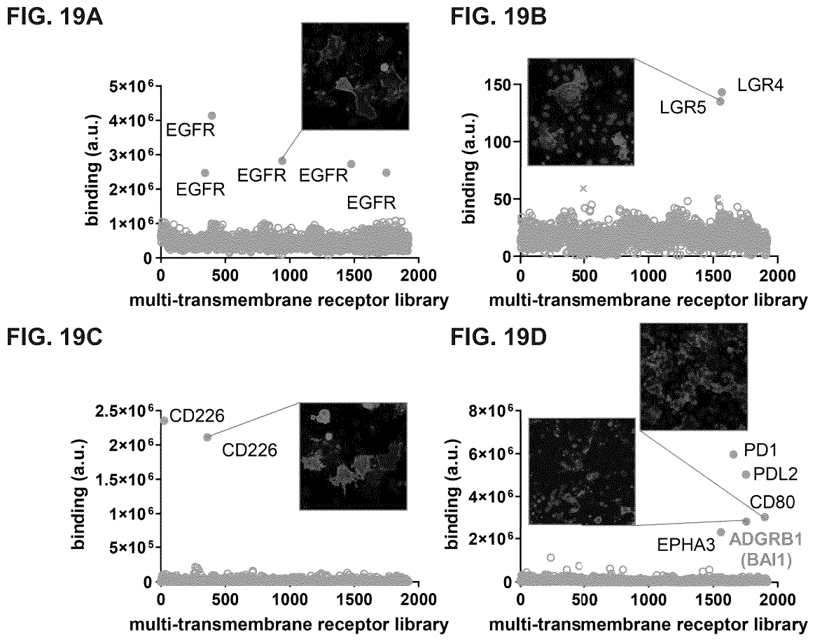

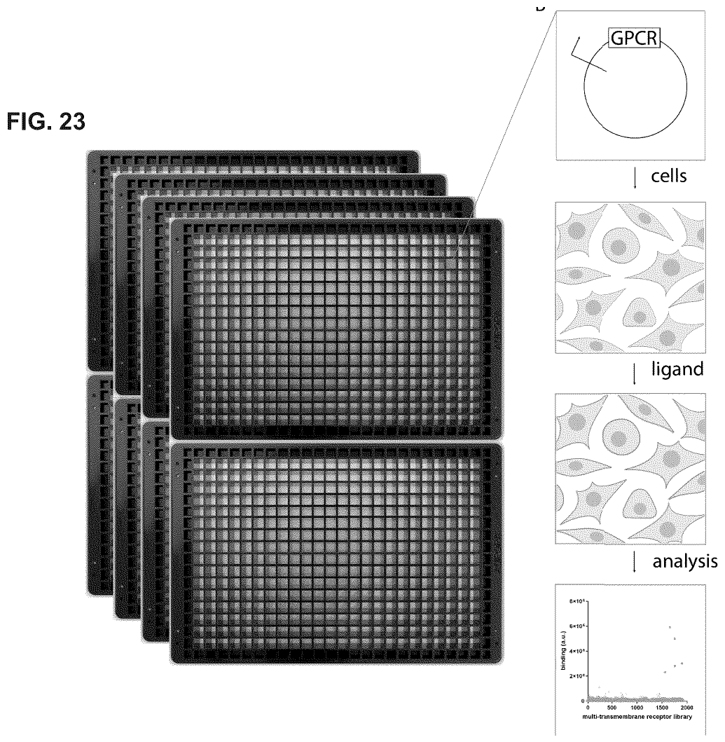

Using this comprehensive library, four fluorescently labelled peptide ligands were screened using high-throughput transfections and high-content imaging (FIG. 23). EGF and RSPO3 were chosen because they have well-characterized receptors. PD-L1 and PVR bind to complex networks of the immunoglobulin superfamily of receptors and are the target of many biological therapeutics (Andrews et al., Nat Immunol, 20: 1425-1434, 2019). EGF-647 bound only to the control EGFR added to each plate as a transfection control (FIG. 19A). R-spondin 3 (RSPO3) was fused to an Avidity AVITAG™ (Avi tag) to allow for biotinylation and tetramerization with an APC labeled streptavidin. This tetramerized RSPO3 bound to its known G protein-coupled receptors, leucine rich repeat GPCR (LGR) 4 and 5 (FIG. 19B). The extracellular domain of the polio virus receptor (PVR) was tetramerized and found to only bind to the control single-transmembrane receptor CD226 that was added to the screen as a control (FIG. 19C).

When the extracellular domain of programmed cell death ligand 1 (PD-L1) was screened as a fluorescent tetramer, it bound to controls and also to adhesion GPCR B1 (ADGRB1) (FIG. 19D). Based on this unexpected finding, a method for mapping the complete interactions of one GPCR against families of potential interacting proteins was developed.

C. A Vesicle-Based Interaction Map for ADGRB1 and Leucine Rich GPCRs

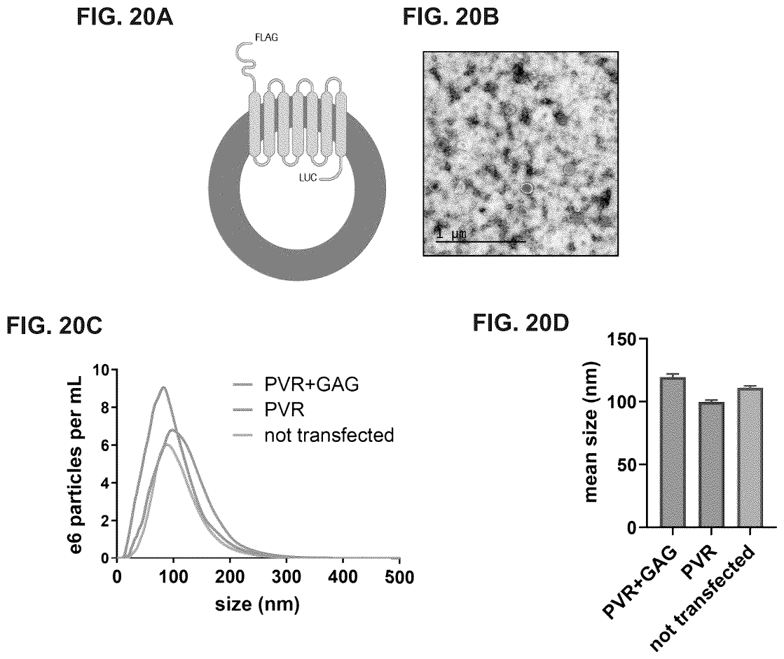

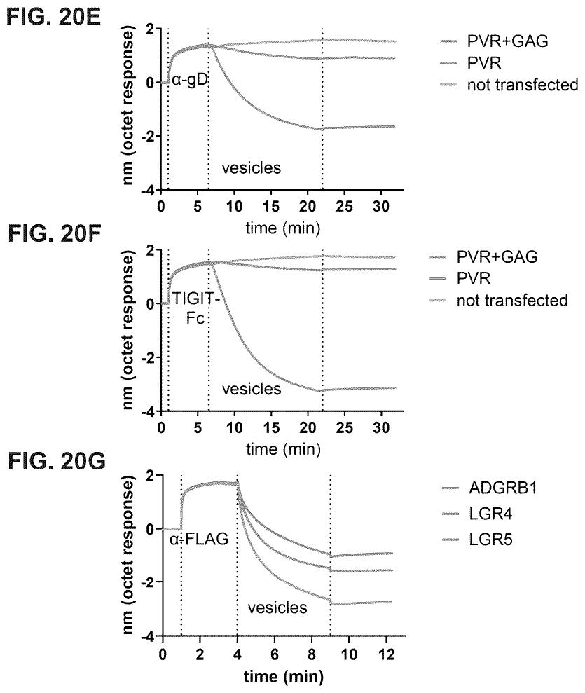

Recombinant extracellular vesicles (rEVs) (Geeurickx et al., Nat. Commun., 10: 1-12, 2019) were used in order to map the interactions of G protein-coupled receptors (GPCRs). rEVs are generated by co-transfecting a receptor of interest along with the GAG protein from HIV, as described in Example 1. GAG stimulates the production of microvesicles and exosomes (Geeurickx et al., Nature Protocols, 16: 603-633, 2021), which have a uniform packaging orientation (FIG. 20A) and size (FIGS. 20B-20D). GAG co-transfection enhanced the packaging of receptors into vesicles (FIGS. 20E and 20F), and GPCRs trafficked efficiently into rEVs (FIG. 20G), as shown using biolayer interferometry (BLI). BLI allows for the detection of vesicles where the association curve is inverted (Cameron et al., Octet® Potency Assay: Development, Qualification and Validation Strategies. Satorius Application Note, 2021), allowing confident determination that the association is with an rEV-sized particle in our BLI readouts.

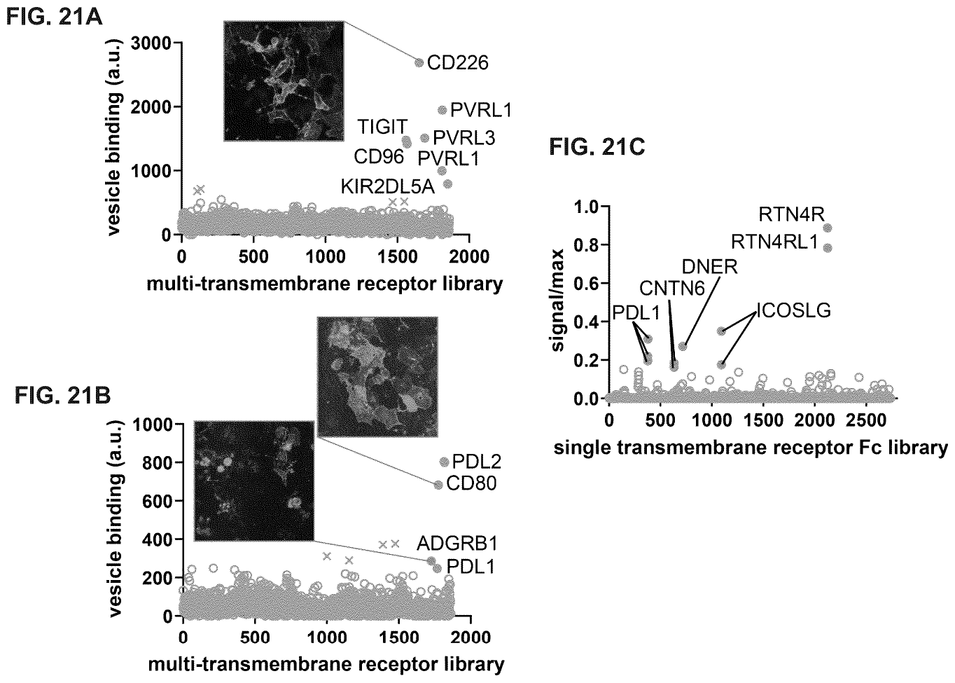

The poliovirus receptor (PVR) and programmed cell death ligand 1 (PD-L1) were packaged into vesicles, along with a fluorescently labelled GAG, and these rEVs were screened against the comprehensive multi-transmembrane library. As with the recombinant proteins shown in FIGS. 18A-18D, PVR packaged into rEVs bound to only the control single-transmembrane receptors (FIG. 21A) and PD-L1 bound to the control receptors as well as ADGRB1 (FIG. 21B).

Next, the recently published (Martinez-Martin et al., Nature Communications, 7: 11473, 2016; Verschueren et al., Cell, 182: 329-344.e19, 2020) avidity-based extracellular interaction library of proteins was adapted to map the interactions of GPCRs packaged into rEVs. Briefly, the library comprises a large collection of Fc-tagged proteins in mammalian expression vectors with a signal sequence for secretion into the media. After transfection, the conditioned media is incubated on Protein A coated white plates, thus capturing the proteins from the media, as described above. For this screen, each GPCR was fused to Rluc8 (Loening et al., Protein Engineering, Design & Selection, 19(9): 391-400, 2006) in order to allow for rapid and sensitive detection of receptor-ligand interactions. GPCRs packaged into rEVs binding to a member of the protein library were detected using luciferase-produced light.

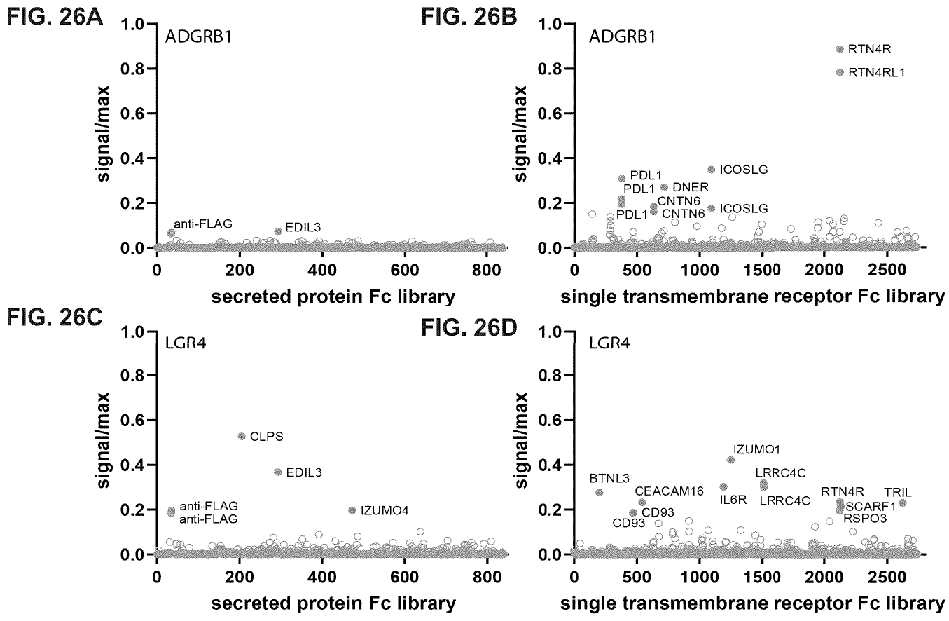

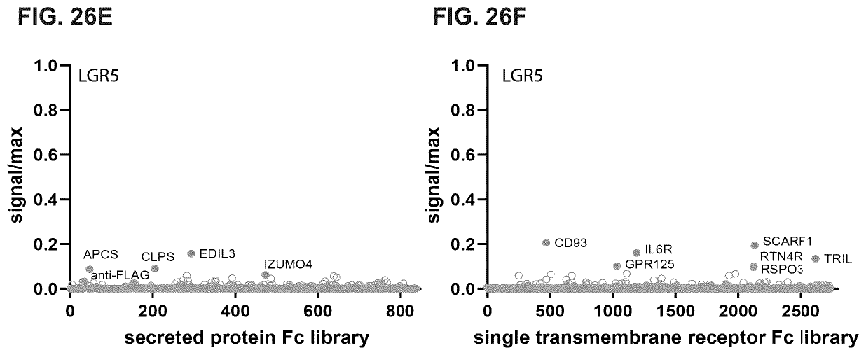

ADGRB1 was screened against the collection of single-transmembrane receptor extracellular domains (STM library) fused to Fc (FIG. 21C). The interactions between ADGRB1 and RTN4R family members (Chong et al., Genome Biology, 19(1): 205, 2018), as well as PD-L1, were confirmed. A number of novel interactions were also uncovered, including ICOSLG, a protein related to PD-L1 (Table 15; Greenwald et al., Annual Review of Immunology, 23: 515-548, 2005). FIGS. 26A-26F show the results of screens for binding of EVs comprising ADGRB1, LGR4, or LGR5 to the STM library or to a library of secreted proteins fused to Fc. Novel interactions identified in these screens are shown in Table 15.

TABLE 15
Interactions identified in screen

| | |
|---|---|
| ADGRB1 | PD-L1 |
| | ICOSLG |
| | DNER |
| | CNTN6 |
| LGR4 | CLPS |
| | EDIL3 |
| | IZUMO4 |
| | IZUMO1 |
| | BTNL3 |
| | CD93 |
| | CEACAM16 |
| | IL-6 |
| | LRRC4C |
| | SCARF1 |
| | TRIL |
| LGR5 | CLPS |
| | EDIL3 |
| | IZUMO4 |
| | CD93 |
| | GPR125 |
| | IL6R |
| | SCARF1 |
| | TRIL |

D. Recombinant Proteins Confirm ADGRB1 Binders Found

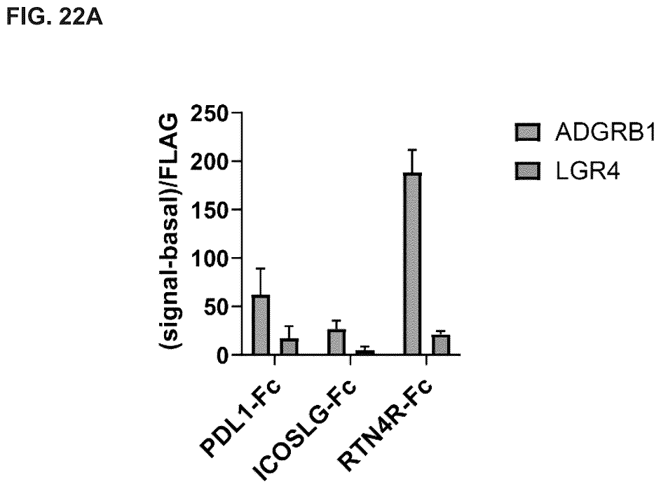

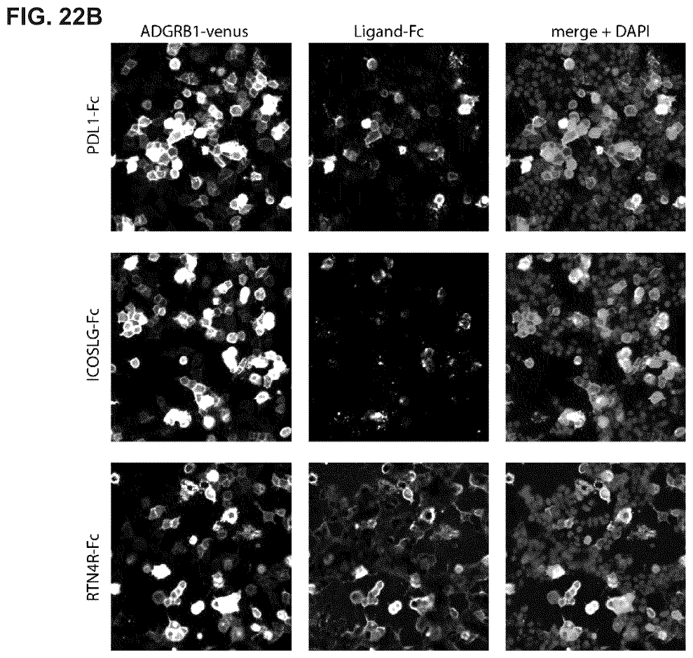

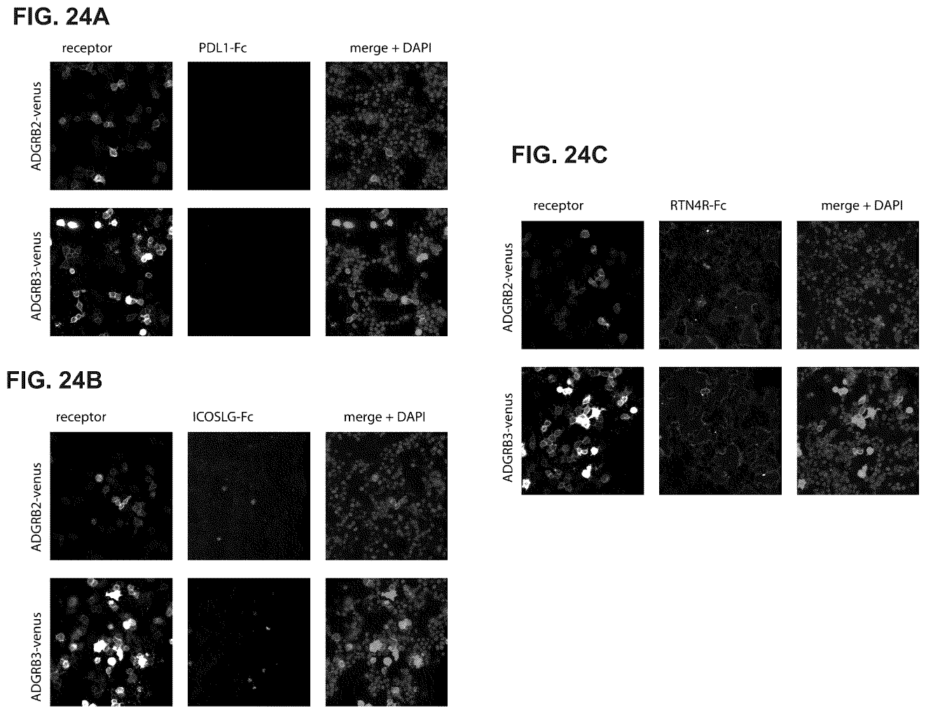

Recombinant proteins were used to confirm some the interactions (FIG. 22A). Cells overexpressing ADGRB1 fused to Venus (FIG. 22B) were treated with recombinant PD-L1, ICOSLG, or RTN4R fused to an Fc tag and robust binding was observed by staining for the Fc tag. This binding was not observed for ADGRB2 or ADGRB3 (FIGS. 24A-24C).

E. Discussion

G protein-coupled receptor (GPCR) interaction mapping is a powerful, yet underdeveloped area of research. In this Example, two interaction-mapping platforms have been adapted to accommodate GPCRs (FIG. 23). The first is a cell-based platform that can be used to uncover novel receptors for orphan ligands. Here, this cell-based platform was used to discover a previously unappreciated receptor for PD-L1 called adhesion GPCR B1 (ADGRB1). Secondly, an avidity-based interaction screen was implemented using GPCRs in recombinant extracellular vesicles (rEVs), as described above. In this way, a GPCR of interest can be screened against libraries of potential interacting partners. Using this vesicle-based platform, ADGRB1 was shown to bind to ICOSLG.

ADGRB1 belongs to a family of 33 adhesion GPCRs that share a remarkable long N-terminus. Adhesion GPCRs are thought to autoproteolyze this long extracellular domain in the Golgi apparatus but remain complexed together at the plasma membrane. Upon binding of ligands, the large extracellular domain dissociates from the seven transmembrane domain, and the remaining short stalk activates the seven transmembrane domain, in a similar way to the protease activated receptor family (Nijmeijer et al., Biochemical Pharmacology, 114: 88-102, 2016). Adhesion GPCRs have been shown to activate G proteins and also recruit arrestins (Kishore et al., The Journal of Biological Chemistry, 291(7): 3385-3394). The large extracellular domain of ADGRB1 binds to lipopolysaccharides and phosphatidylserine, and receptor activation is believed to drive engulfment of bacterial cells and apoptotic cells (Park et al., Nature, 450(7168): 430-434, 2007; Das et al., The FASEB Journal, 28(5): 2214-2224). ADGRB1 expression on macrophages was previously demonstrated (Park et al., Nature, 450(7168): 430-434, 2007); however, that observation has recently been challenged (Hsiao et al., Frontiers in Immunology, 10: 962, 2019). ADGRB1 is also known to be a tumor suppressor gene that is down-regulated in cancer cells (Zhu et al., Cancer Cell, 33(6): 1004-1016e15, 2018).

It is interesting to note that ADGRB1 binding to two of the classical ligands for T cell activation (PD-L1 and ICOSLG) was observed. However, these ligands are known to have opposing effects on T cell activation, where PD-L1 inhibits T cells and ICOSLG activates them (Greenwald et al., Annual Review of Immunology, 23: 515-548, 2005). One of the ways that tumors evade T cells is by overexpressing PD-L1, which drives the silencing of the T cells, thus allowing tumors to evade the immune system. Indeed, PD-1/PD-L1 has been targeted by biological drugs to disinhibit T cell silencing and is a proven clinically effective immunotherapy for cancer (Lee et al., Scientific Reports, 7(1): 5532, 2017). The interaction of ADGRB1 with PD-L1 and ICOSLG may offer a new avenue for drug development, as PD-1/PD-L1 blockade can be ineffective (Lee et al., Frontiers in Pharmacology, 12: 681320, 2021).

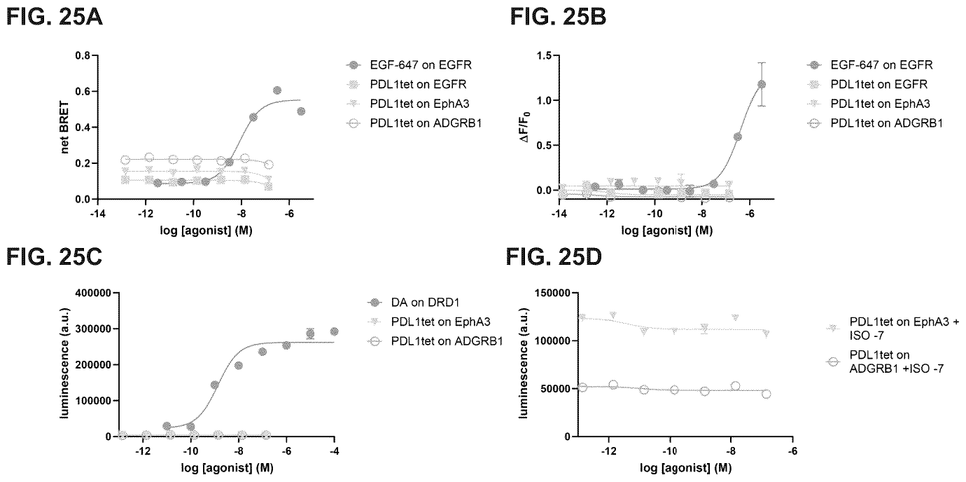

One unanswered question is the impact of the interaction of ADGRB1 with PD-L1 and ICOSLG on signaling. It has been difficult to develop an ADGRB1-based signaling model in heterologous cell lines (FIGS. 25A-25D). Recently, another adhesion GPCR family member was shown to activate G_{αi} in response to N-termini binding and also by treatment with cholesterols, which bind directly to the seven transmembrane domain (Ping et al., Nature, 589(7843), 620-626, 2021). Previous work has shown that ADGRB1 is not cleaved in HEK cells (Araç et al., The EMBO Journal, 31(6): 1364-1378, 2012), and also that ADGRB1 signals through a non-canonical effector called ELMO (Park et al., Nature, 450(7168): 430-434, 2007). Activation of ADGRB1 may be only possible in relevant cell lines or in vivo.

GPCRs are a critical target for many drug development programs. Many small molecule agents against GPCRs have successfully been brought to the clinic. However, novel drug modalities, such as biologicals, have lagged behind small molecules for GPCR drug development. Novel modalities, especially antibody-based biologicals, offer a robust way to target extracellular protein interactions and also to modulate receptor activation. Here, methods for mapping GPCR extracellular interactions were developed and new interactions that have implications for cancer immunotherapies were found.

F. Materials and Methods

Cell Culture

HEK 293T and COS7 cells were maintained in DMEM+10% FBS, 10 mM HEPES pH 7.4, and Penicilin-Streptomycin (100 U/mL). EXPI293F™ cells were cultured in EXPI293™ expression media shaking at 150 RPM.

Multi-Transmembrane Receptor Library Generation

Receptors were cloned into either the pT-Rex-DEST31 plasmid (Invitrogen) or the pRK plasmid (Genentech) and sequence verified. 100 ng of receptor DNA (at 10 ng/μL for 10 μL/well) was plated into each well of a 384 well black Aurora Microplate (cat #ABC2-312-1B-PDL). DNA-printed Aurora plates were sealed and stored at −20° C. until the day of the experiment.

On the day of the transfection, DNA-printed Aurora plates were thawed at room temperature and spun down. 20 μL of Opti-MEM™ (Thermo Fisher, cat #11058021) with LIPOFECTAMINE™ LTX diluted 1:0.0072 and PLUS™ reagent diluted 1:0.0024 (Thermo Fisher, cat #15338100) was added to each well of the 384 well plate and incubated for 20 minutes at 37° C. with 5% CO₂. Afterwards, 20 μL of COS7 cells, diluted at 150,000 cells/mL in DMEM+10% FBS, HEPES and P/S, were added to each well and incubated at 37° C. with 5% CO₂ for 48 hours.

Receptor Expression Analysis

After 48 hours, expression was verified by receptor fluorescence. Cells were starved in Opti-MEM™+5% BSA for 45 minutes at 37° C. with 5% CO₂. Next, rabbit anti-HIS antibody (Cell Signaling, cat #2365) or mouse anti-FLAG antibody (Sigma, cat #F3165) were diluted 1:1000 in Opti-MEM™+5% BSA and incubated on cells for 45 minutes at 4° C. Cells were then washed in PBS+Ca/Mg and fixed in 4% PFA for 20 minutes at room temperature, washed again in PBS+Ca/Mg, stained with a rabbit or mouse Alexa-647 secondary antibody diluted 1:1000 in Opti-MEM™+5% BSA at room temperature. The cells were then washed, then DAPI stained (Thermo Fisher, cat #62248) at 1 μg/mL for 20 minutes at room temperature, washed again. and stored in PBS+Ca/Mg at 4° C. until the day of imaging.

Receptor expression was evaluated by capturing two imaging fields using a 10× objective on an IN Cell Analyzer 6000 (GE Healthcare) with preset filters for DAPI, GFP and Cy5. The IN Cell Analyzer 6000 Development software was used to draw regions of interest for the DAPI, GFP, and Cy5 channels and count the total number of objects as well as the pixel density for the entire field. Total expression is expressed as the GFP channel divided by the total number of cells (DAPI counts). Surface expression is expressed as the antibody channel (Cy5) divided by total number of cells.

Cell-Based Interaction Screening

Similar to the receptor expression analysis, cells were blocked 48 hours after transfection in Opti-MEM™+5% BSA. Instead of primary antibodies, cells were treated with 100 nM tetramerized ligand diluted in Opti-MEM™+5% BSA for 45 minutes at 4° C. Tetramers were prepared as previously described (Verschueren et al., Cell, 182(2): 329-344.e19, 2020) with minor modifications. Briefly, the total mass of streptavidin-APC (Agilent, PJ27S) and the biotinylated protein of interest (RSPO3 and PVR were produced in-house, PD-L1 was purchased from Bio-Techne, cat #AVI156) was calculated. The total volume of protein needed was aliquoted and the volume of streptavidin was added in four steps with 10-minute incubations at room temperature in between. After the final addition and incubation, the tetramer was diluted to a final concentration of 100 nM in Opti-MEM™+5% BSA. For EGF-647, the fluorescent protein was purchased from Thermo Fisher (cat #E35351) and diluted to 100 nM in Opti-MEM™+5% BSA. Cells were then washed with PBS+Ca/Mg and fixed in 4% PFA for 20 minutes at room temperature, washed again, and DAPI stained (Thermo Fisher, cat #62248) and stored at 4° C. in PBS+Ca/Mg until the day of imaging.

For the vesicle-based interaction screening, vesicles were added instead of tetramerized ligand, and GAG was fused to Neon Green.

Imaging was done as described above for the receptor expression analysis.

Recombinant Extracellular Vesicle Preparations

100 mL of EXPI293F™ cells were transfected with 100 μg of DNA, with a split of 50 μg of GAG DNA and 50 μg of receptor. Seven days after transfection, the cells were spun down and media was filtered through a 0.2 micron filter. Protease inhibitor (Roche) was added to the filtered media and it was spun at a slow speed of 2,000×g for 30 minutes. Next, the media was spun at 100,000×g for 90 minutes. Vesicle pellets were reconstituted in PBS+Ca/Mg and stored until the day of experiments.

Electron Microscopy

Electron microscopy was performed as described herein. The suspension of vesicles was adsorbed for 15 minutes to the surface of formvar and carbon coated TEM grids. After a short rinse with distilled water, the sample was stained with 2% phosphotungstic acid (PTA) for EV prep cleanup for 60 seconds and then air dried. Samples were then washed with PBS for 15 minutes, with water for 1 minute, and stained with 1% uranyl acetate for 1 minute before being blotted and air dried. Imaging was done with a JEOL JEM-1400 transmission electron microscope (TEM) and a GATAN ULTRASCAN® 1000 CCD camera at magnifications from 5000× to 50000×. Scale bars are indicated in the images.

### NanoSight

Vesicles were diluted in clean PBS and NanoSight (Malvern Panalytical) data was collected and averaged for three runs with the clean PBS used as a baseline.

Biolayer Interferometry

Vesicles were diluted into PBS and the gD antibody (abcam, cat #ab6507) and FLAG antibody (Sigma, cat #F3165) were diluted 1:10 in PBS; TIGIT-Fc (Bio-techne cat #7898-TGB) was diluted to 100 μg/mL. AMC tips (Sartorius, cat #18-5088) or AHC tips (cat #18-5060) were used to capture the antibodies or Fc tag, respectively.

Vesicle-Based Interaction Screening

Vesicle-based interaction screens were performed as described above. For the control proteins and follow-up experiments, recombinant proteins and antibodies were added to empty wells of the white 384 well protein A coated plates (Thermo Fisher, cat #NCI15133) and allowed to incubate for at least 24 hours at 4° C.

Ligand Binding

For ligand binding, the same protocol was used as for the interaction screening, except that receptors of interest were transiently overexpressed in HEK cells using calcium phosphate transfections, then plated onto Aurora imaging plates. Instead of tetramerized ligand, Fc-fused proteins (BIO-TECHNE®, PD-L1 cat #AVI156, ICOSLG cat #AVI165, and RTN4R cat #1208-NG) were incubated on cells at a concentration of 10 μg/mL (˜70 nM for each dimer). Anti-human Fc fused to Alexa-647 was then used as a secondary antibody, and images were captured on an IN Cell Analyzer 6000.

### Example 7. Cell-State Profiling Using rEV-Based RDIMIS in Different Cell Lines

The above Examples demonstrate that rEVs can be used to identify and characterize interactions with receptors of interest. In addition, a number of binding partners that interact with rEVs in general were identified (see, e.g., Example 4). These interactions may capture a snapshot of the state of the cells of origin (parent cells) for the rEVs, as receptors of interest and many other membrane and cytoplasmic proteins appear to be incorporated into rEVs at levels proportional to their expression levels in the parent cells.

It is therefore hypothesized that rEVs can capture cell surface proteins (e.g., can capture a representative sample of the cell surface proteins at a given time point) and RDIMIS can thus be used to profile interactions that are relevant for cells with different cell states. Specifically, rEVs may be used as a proxy for cellular interactions with the rEVs' parent cells and may capture important interactions that arise due to, e.g., disease-specific stimulation, differentiation, and cell-type differences. One advantage of this approach is that it captures not only the aggregate binding behavior of cell-surface proteins, including complexes and complicated interaction dynamics, but also interactions that depend on non-protein components like glycosylation marks and lipids.

Methods

Stable cell lines that express a vesicle budding factor and a readout for RDIMIS (e.g., a vesicle budding factor attached to a readout for RDIMIS, e.g., gag-Renilla luciferase (gag-RLuc)) are generated for cell lines including but not limited to: immune cell lines representing T-cells (JURKATs), B-cells (BJABs) and monocytes (THP1s); neuronal cell lines; and fibroblast cell lines. rEVs are generated from these cell lines as described in Example 1.

The cell lines and rEVs generated therefrom are implemented for:
1. Characterization of differences in the interaction profiles of rEVs from different cell lines representative of different cell types; and
2. Characterization of the interaction profiles of rEVs from the same cell line over time (e.g., at time points before and after the addition of stimuli, before and after inducing signaling, and/or before and after induction of a disease-related state (e.g., immune exhaustion) or at two or more time points in a differentiation pathway or profile). In one approach, rEVs are collected at select time points, e.g., after stimuli are applied. In another approach, the cell line is modified such that the budding factor is present (e.g., expressed) only during selected periods. Expression of the budding factor may be controlled using:
(a) an inducible promoter that relieves or suppresses the expression of the budding factor after the addition of a small molecule (e.g., a cell-permeable small molecule), e.g., the T-REX™ System;
(b) a small molecule-induced degradation system in which a protein (e.g., the budding factor) is rapidly degraded upon induction (e.g., the TIR1 auxin inducible degron (AID) system); or
(c) a small molecule-induced stabilization system in which a protein (e.g., the budding factor) comprises a degradation domain and the protein is protected from degradation upon induction (e.g., the Shield-1-FKBP system).

Integration of constructs can be performed, e.g., by using CRISPR-Cas9 or genome engineering techniques to insert the construct into a safe-harbor locus. Alternatively, methods of random integration such as the PiggyBac Transposon System (SBI) may be used.

### Example 8. Use of EVs to Measure Membrane Protein-Associated Enzymatic Activities

The above Examples demonstrate that EVs can be used to display membrane proteins and membrane-associated proteins in the context of a membrane. This includes proteins with enzymatic activity, such as peptidases, proteases, and phosphatases. The activities of such proteins can be detected and assayed on EVs, thus allowing biochemical characterization of the enzymatic effects of membrane proteins in or on membranes using assays usually designed for recombinant proteins.

In one approach, rEVs comprising proteins with enzymatic activity are used in enzymatic assays for membrane proteins. In another approach, rEVs comprising proteins with enzymatic activity are used in drug discovery for, e.g., molecular glues, wherein peptidases, proteases, phosphatases, and kinases can be recruited to another membrane protein on the same EV or to inhibit specific membrane-bound enzymatic activities.

Peptidase Activity Assay

As a proof of concept, it was tested whether the peptidase activity of Carboxypeptidase M (CPM) could be detected and assayed on vesicles. Either full-length (FL) or gD-GPI (gD) CPM was expressed in vesicles.

An assay for peptidase activity was performed as follows. This assay was modified from the manufacturer's provided protocol for use of Recombinant Human Carboxypeptidase M Protein (R&D SYSTEMS®).
1. EVs were diluted. 200 μL Assay Buffer was added, and further reagents were added as listed below.
2. The substrate Bz-Ala-Arg-OH (50 mM stock solution) was diluted to 1 mM in Assay Buffer (126 μL in 6300 μL Assay Buffer)
3. 150 μL of EVs and 150 μL 1 mM substrate were mixed. Controls containing 150 μL of 1 mM substrate only were prepared.
4. Reactions were incubated for 10 minutes at room temperature.
5. Reactions were stopped by adding 300 μL of a solution containing 15 mM o-PA in 0.2 M NaOH containing 0.1% (v/v) 2-Mercaptoethanol and mixing well. (1260 μL 2M NaOH+12.6 μL Bme+507 μL o-PA+10.8 mL water)
6. 150 μL of EVs was added to controls after stopping the reaction.
7. All samples were incubated for 10 minutes at room temperature.
8. 195 μL of the incubated samples were loaded in triplicate into a plate.
9. Remaining EV solution was also added to plates too to confirm no substrate background.
10. Samples were read at excitation and emission wavelengths of 330 nm and 450 nm (top read), respectively, in endpoint mode.

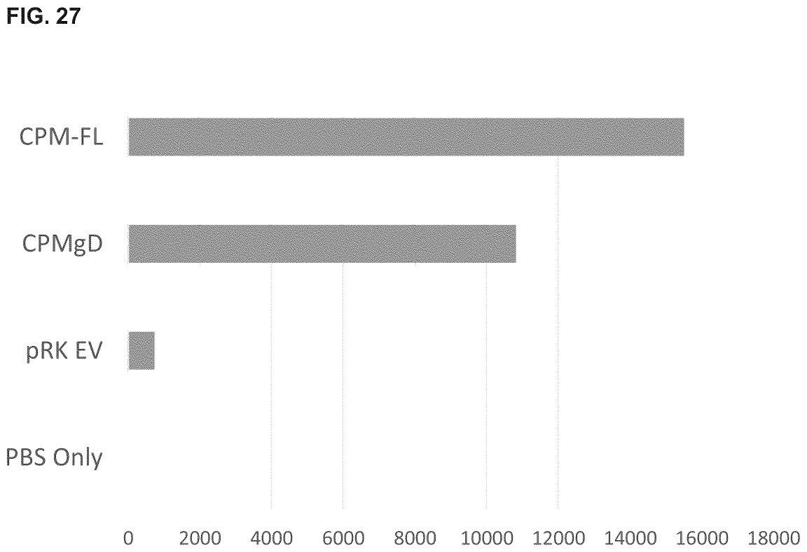

Results of the assay are shown in FIG. 27. Robust peptidase activity on CPM-expressing EVs was detected (FIG. 27). This activity was significantly greater than that observed in EVs not overexpressing CPM (pRK EV; vector control showing background levels of peptidase activity). This activity was more readily detectable compared to peptidase activity observed using recombinant protein.

Kinase Activity Assay

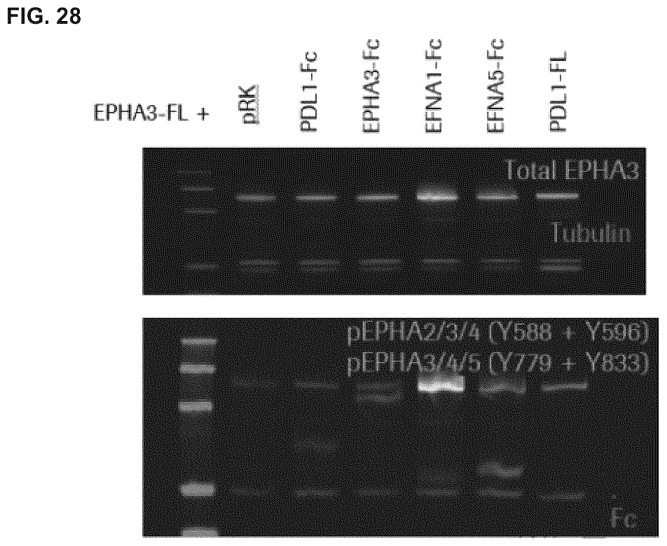

It was also demonstrated that signs of kinase activity can be detected on vesicles: as shown in FIG. 28, co-expression of EPHA3 EVs with the EPHA3 ligands EFNA1-Fc and EFNA5-Fc enhanced the amount of EPHA3 phosphorylated species detected in vesicles. EVs were lysed and put into sample buffer and run on an acrylamide gradient gel to separate the protein species. They were then blotted with EPHA3, phospho-specifics, tubulin antibodies, and primary antibodies and detected with either fluorescently tagged secondary antibodies (680 and 800 nm LI-COR® dyes) or anti-human antibodies to detect the Fc region on the various proteins being expressed. The blot was then imaged on a LI-COR® instrument. The same samples were loaded separately onto 2 different gels corresponding to the top and bottom images.

### Example 9. Use of Generic EV Binders for Vesicle Purification

The gold standard for EV purification to date is ultracentrifugation, which is a long and cumbersome process. While there are some affinity-based methods to purify specific EVs, they are either too dirty, receptor-specific, or are designed for too small a scale to be useful for RDIMIS screening and other applications that require large quantities of EVs.

As described in Example 4, generic vesicle binders have been identified. These generic vesicle binders can be used to affinity purify EVs directly from conditioned medium. The generic vesicle binders have been ranked by ability to bind to the pure recombinant EVs described herein (Table 9), and they can be used to purify human EVs in general. In one approach, the binding conditions described in the above examples are reproduced on a column. In one approach, the column comprises Protein A-functionalized beads (e.g., is a PROTEIN A SEPHAROSE® column). Conditioned media containing one or more of the top generic EV binders (Table 9) modified to comprise an Fc region (e.g., a human Fc region) is flowed over the column, the column is washed, and then conditioned media containing EVs is flowed over the column. The EVs are then eluted using appropriate methods, e.g., harsh washes like high salt and/or specific ligands like sialoglycans for the SIGLEC family of proteins. A quick ultracentrifugation-based cleanup may be performed if necessary.

1. A method for identifying a protein-protein interaction, the method comprising:
(a) providing a collection of target polypeptides that are immobilized on one or more solid surfaces;
(b) contacting the collection of step (a) with a biological vesicle (BV) comprising a heterologous membrane-associated protein and a membrane-budding agent under conditions permitting the binding of the heterologous membrane-associated protein and at least one of the target polypeptides, wherein the heterologous membrane-associated protein is expressed at or above a threshold level on the surface of the BV; and
(c) detecting an interaction between the heterologous membrane-associated protein and the at least one target polypeptide, thereby identifying a protein-protein interaction.

2. The method of claim 1, wherein one or more of the target polypeptides is immobilized to a distinct location on the one or more solid surfaces, and wherein detecting an interaction comprises detecting a signal at a location on the solid surface that is above a threshold level.

3. (canceled)

4. The method of claim 1, wherein the membrane-budding agent is selected from the group consisting of a HIV gag protein, Acyl.Hrs, ARRDC1, and ARF6.

5-10. (canceled)

11. The method of claim 1, wherein the interaction is a transient interaction or a low-affinity interaction.

12. (canceled)

13. The method of claim 1, wherein:
(i) the heterologous membrane-associated protein is a full-length protein; or
(ii) the heterologous membrane-associated protein comprises a protein fragment, a tag, and an anchor.

14-15. (canceled)

16. The method of claim 13, wherein the anchor is a glycosylphosphatidyl-inositol (GPI) polypeptide and/or the tag is a glycoprotein D (gD) polypeptide.

17-31. (canceled)

32. A BV comprising (a) a heterologous membrane-associated protein comprising a protein fragment, a tag, and an anchor, wherein the heterologous membrane-associated protein is present on the outer face of the BV and (b) a membrane-budding agent.

33. The BV of claim 32, wherein the BV is produced by a process comprising (i) providing a parent cell that has been modified to express the heterologous membrane-associated protein and the membrane-budding agent and (ii) isolating the BV from the parent cell.

34. The BV of claim 32, wherein the membrane-budding agent is selected from the group consisting of a HIV gag protein, Acyl.Hrs, ARRDC1, and ARF6.

35. (canceled)

36. The BV of claim 32, wherein the anchor is a GPI polypeptide and/or the tag is a gD polypeptide.

37-55. (canceled)

56. A method of identifying a modulator of the interaction between a protein of Table 1 and a protein of Table 2, the method comprising:
(a) providing a candidate modulator;
(b) contacting a protein of Table 1 with a protein of Table 2 in the presence or absence of the candidate modulator under conditions permitting the binding of the protein of Table 1 to the protein of Table 2, wherein the protein of Table 1 and the protein of Table 2 are reported to interact in Table 3; and
(c) measuring the binding of the protein of Table 1 to the protein of Table 2, wherein an increase or decrease in binding in the presence of the candidate modulator relative to binding in the absence of the candidate modulator identifies the candidate modulator as a modulator of the interaction between the protein of Table 1 and the protein of Table 2.

57-81. (canceled)

82. A method for identifying a biological vesicle (BV) having an altered binding profile, the method comprising:
(a) providing a collection of target polypeptides that are immobilized on one or more solid surfaces;
(b) contacting the collection of step (a) with a BV of interest;
(c) detecting an interaction between the BV of interest and the at least one target polypeptide, thereby identifying an interaction profile; and
(d) comparing the interaction profile of the BV of interest to the interaction profile of a control BV, wherein a difference between the interaction profile of the BV of interest and the interaction profile of the control BV identifies the BV of interest as one having an altered binding profile.

83-88. (canceled)

89. The method of claim 82, wherein the BV of interest is derived from a sample from a subject.

90. The method of claim 89, wherein the BV of interest and the control BV are derived from different tissues or different cell types.

91. The method of claim 89, wherein the BV of interest is derived from a diseased tissue and the control BV is derived from healthy tissue.

92. A method of identifying a modulator of the interaction between a protein of Table 5 and a protein of Table 6, the method comprising:
(a) providing a candidate modulator;
(b) contacting a protein of Table 5 with a protein of Table 6 in the presence or absence of the candidate modulator under conditions permitting the binding of the protein of Table 5 to the protein of Table 6, wherein the protein of Table 5 and the protein of Table 6 are reported to interact in Table 7; and
(c) measuring the binding of the protein of Table 5 to the protein of Table 6, wherein an increase or decrease in binding in the presence of the candidate modulator relative to binding in the absence of the candidate modulator identifies the candidate modulator as a modulator of the interaction between the protein of Table 5 and the protein of Table 6.

93-130. (canceled)

131. A method for characterizing an interaction profile of a cell line that has been modified to comprise a membrane-budding agent, the method comprising characterizing an interaction profile of a BV produced by the cell line.

132. (canceled)

133. A method for identifying a change in the interaction profile of a cell line that has been modified to comprise a membrane-budding agent, the method comprising:
(a) characterizing an interaction profile of a BV produced by the cell line at a first time point;
(b) characterizing an interaction profile of a BV produced by the cell line at a second time point; and
(c) comparing the interaction profile of the BV produced at the first time point to that of the BV produced at the second time point, wherein a difference between the interaction profile of the BV produced at the first time point and that of the BV produced at the second time point identifies a change in the interaction profile of the cell line.

134-136. (canceled)

137. The method of claim 133, wherein the method comprises exposing the cell line to a stimulus following the first time point and before the second time point.

138-143. (canceled)

144. A method for identifying a difference in the interaction profiles of two cell lines that have been modified to comprise a membrane-budding agent, the method comprising:
(a) characterizing an interaction profile of a BV produced by the first cell line;
(b) characterizing an interaction profile of a BV produced by the second cell line; and
(c) comparing the interaction profile of the BV produced at the first cell line to that of the BV produced by the second cell line, wherein a difference between the interaction profile of the BV produced by the first cell line and that of the BV produced by the second cell line identifies a difference in the surface protein profiles of two cell lines.

145-155. (canceled)

156. The method of claim 1, wherein the BV is produced by a process comprising (i) providing a parent cell line that has been modified to express the membrane-budding agent under inducible control; (ii) inducing expression of the membrane-budding agent, and (iii) isolating the BV from the parent cell line.

157-175. (canceled)


SEQUENCE LISTING (1 sequence)

SEQ ID NO: 1 - protein, 500 aa, Human immunodeficiency virus
MGARASVLSGGELDRWEKIRLRPGGKKKYKLKHIVWASRELERFAVNPGLLETSEGCRQILGQLQPSLQTGSEELRSLYNTVATLYCVHQRIEIKDTKEALDKIEEEQNKSKKKAQQAAADTGHSNQVSQNYPIVQNIQGQMVHQAISPRTLNAWVKVVEEKAFSPEVIPMFSALSEGATPQDLNTMLNTVGGHQAAMQMLKETINEEAAEWDRVHPVHAGPIAPGQMREPRGSDIAGTTSTLQEQIGWMTHNPPIPVGEIYKRWIILGLNKIVRMYSPTSILDIRQGPKEPFRDYVDRFYKTLRAEQASQEVKNWMTETLLVQNANPDCKTILKALGPGATLEEMMTACQGVGGPGHKARVLAEAMSQVTNPATIMIQKGNFRNQRKTVKCFNCGKEGHIAKNCRAPRKKGCWKCGKEGHQMKDCTERQANFLGKIWPSHKGRPGNFLQSRPEPTAPPEESFRFGEETTTPSQKQEPIDKELYPLASLRSLFGSDPSSQ